(19) United States
(12) Patent Application Publication
(10) Pub. No.: US 2023/0174614 A1
(43) Pub. Date: Jun. 8, 2023

(54) MULTISPECIFIC ANTI-FLT3 CHIMERIC ANTIGEN RECEPTORS

(71) Applicants: Terry J FRY, Denver, CO (US); The Regents of the University of Colorado, A Body Corporate, Denver, CO (US)

(72) Inventor: Terry J. FRY, Denver, CO (US)

(21) Appl. No.: 17/997,575

(22) PCT Filed: Apr. 30, 2021
(86) PCT No.: PCT/US2021/030241

§ 371 Date: Oct. 31, 2022

Related U.S. Application Data
(60) Provisional application No. 63/018,010, filed on Apr. 30, 2020.

(51) Int. Cl. C07K 14/705; A61P 35/02; C12N 15/63; A61K 35/17

(52) U.S. Cl. CPC C07K 14/70503; A61P 35/02; C12N 15/63; A61K 35/17

(57) ABSTRACT

The present disclosure provides multispecific chimeric antigen receptors (CARs) having antigenic specificity for FLT3 and CD19, FLT3 and CD22, FLT3 and CD123 or FLT3 and CD33. Nucleic acids, expression constructs, recombinant expression vectors, host cells, populations of cells, and pharmaceutical compositions relating to the multispecific CARs are disclosed. Methods of treating or preventing ALL, AML and/or other forms of cancer in a mammal are also disclosed.

### RELATED APPLICATIONS

This application claims priority to, and the benefit of, U.S. Provisional Application No. 63/018,010 filed on Apr. 30, 2020, the contents of which are incorporated herein by reference in their entireties.

### GOVERNMENT SUPPORT

This invention was made with government support under Grant No. 1U01CA232486-01 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Apr. 30, 2021, is named “UNCO-030_001WO_SeqList.txt” and is about 199 KB in size.

### FILED OF THE DISCLOSURE

Embodiments disclosed herein provide multispecific and/or multivalent chimeric antigen receptors and constructs encoding the same. Also provided are recombinant expression vectors encoding the multispecific and/or multivalent chimeric antigen receptors and host cells expressing the multispecific and/or multivalent chimeric antigen receptors. Other embodiments disclosed herein provide CAR expression constructs comprising two complete CAR constructs connected via a cleavable linker and host cells comprising these CAR expression constructs. Methods for treating acute lymphoblastic leukemia and acute myeloid leukemia are also provided.

### BACKGROUND

Induction chemotherapy, post-induction consolidation and intensification, followed by prolonged maintenance has been developed over decades as the optimal approach to achieve cure in a high percentage of children with B-cell acute lymphoblastic leukemia (B-ALL). However, adults fare less well than children despite the application of this therapeutic approach, and children with relapsed/refractory ALL remain a challenge. Further, all patients experience long-term treatment-induced morbidity.

Outcomes for adults and children with acute myeloid leukemia (AML) are far inferior despite maximally intensive chemotherapy and allogeneic hematopoietic stem cell transplantation (HSCT). Imminent need thus exists for new targeted immunotherapies for AML, particularly for those patients with chemotherapy-resistant disease.

Chimeric antigen-receptor expressing T-cells (CART) have proven highly successful at inducing remissions in 80-90% of patients with relapsed/refractory ALL, resulting in FDA approval of CD19 CART for children, adolescents and young adults (AYAs) in second or greater relapse of B-ALL. A Children's Oncology Group trial testing CD19 CART as a definitive post-induction therapy without HSCT in children with ‘ultra-high-risk’ B-ALL is in development, representing a major conceptual paradigm shift. Although durable remissions are possible in relapsed/refractory patients when CD19 CART is administered without consolidative HSCT, emerging data indicates that up to 50% of children and AYAs will relapse, most within a year. The most common cause of relapse is loss of the targeted epitope of the CD19 surface protein. Strategies to overcome therapeutic resistance to CD19 CART are clearly needed, as it is likely that resistance to successful CART therapy will occur in other malignancies, in addition to AML.

Approximately 25% of children and AYAs with AML have alterations fms-ike tyrosine kinase 3 (FLT3), either via internal tandem duplication (ITD) or tyrosine kinase domain (TKD) point mutations. These patients also have extremely high relapse risk and are commonly treated with intensive chemotherapy followed by HSCT. Molecularly targeted agents that inhibit FLT3 have demonstrated activity in adult patients but are not curative. Similarly, infants with KMT2A (MLL)-R B-ALL (which overexpresses wild-type FLT3) experience significant toxicity from intensive chemotherapy yet have persistently dismal 20-30% event-free survival (EFS) at 5 years. Adults with KMT2A-R ALL or FLT3-ITD AML fare similarly poorly with <50% 5-year EFS despite intensive chemotherapy and often hematopoietic stem cell transplantation.

New treatments are therefore needed to prevent relapse and to improve long-term cures in children and adults with these high-risk leukemias. Given the extremely high relapse risk and dismal outcomes of patients with Ph-like ALL, KMT2A-R ALL, and FLT3-ITD AML, patients with these less common genetic subtypes will be heavily enriched in relapse trials testing CARTs or other novel agents. Based on emerging data, these leukemias are also the most likely to develop immunotherapeutic resistance via lineage switch or compensatory signaling upregulation.

### SUMMARY

The present disclosure provides a bivalent chimeric antigen receptor (CAR) comprising: a first anti-FLT3 antigen binding domain; a second antigen binding domain selected from the group consisting of: an anti-CD19 antigen binding domain, an anti-CD22 antigen binding domain, an anti-CD33 antigen binding domain and an anti-CD123 binding domain; at least one linker domain; a transmembrane domain; and an intracellular T-cell signaling domain.

A first anti-FLT3 antigen binding domain can comprise: an anti-FLT3 single chain variable fragment (scFv) NC7 variable heavy chain fragment (SEQ ID NO: 16) and variable light chain fragment (SEQ ID NO: 17); an anti-FLT3 single chain variable fragment (scFv) m1006 variable heavy chain fragment (SEQ ID NO: 82) and variable light chain fragment (SEQ ID NO: 83); or an anti-FLT3 single chain variable fragment (scFv) m1007 variable heavy chain fragment (SEQ ID NO: 85) and variable light chain fragment (SEQ ID NO: 86).

An anti-CD19 antigen binding domain can comprise an anti-CD19 scFv FMC63 variable heavy chain fragment (SEQ ID NO: 33) and variable light chain fragment (SEQ ID NO: 34).

An anti-CD22 antigen binding domain can comprise anti-CD22 scFV m971 variable heavy chain fragment (SEQ ID NO: 49) and variable light chain fragment (SEQ ID NO: 50).

An anti-CD33 antigen binding domain can comprise: an anti-CD33 scFv Hum 195 variable heavy chain fragment (SEQ ID NO: 51) and variable light chain fragment (SEQ ID NO: 52); or an anti-CD33 scFc h-p67.6 variable heavy chain fragment (SEQ ID NO: 55) and variable light chain fragment (SEQ ID NO: 56).

An anti-CD123 antigen binding domain can comprise: an anti-CD123 scFv 26292 variable heavy chain fragment (SEQ ID NO: 88) and variable light chain fragment (SEQ ID NO: 92); an anti-CD123 scFv 32701 variable heavy chain fragment (SEQ ID NO: 96) and variable light chain fragment (SEQ ID NO: 100); an anti-CD123 scFv 32716 variable heavy chain fragment (SEQ ID NO: 104) and variable light chain fragment (SEQ ID NO: 108); or an anti-CD123 scFv 32703 variable heavy chain fragment (SEQ ID NO: 112) and variable light chain fragment (SEQ ID NO: 116).

A first anti-FLT3 antigen binding domain and a second antigen binding domain can be arranged in tandem. The first anti-FLT3 antigen binding domain can be proximal to the transmembrane domain. The second antigen binding domain can be proximal to the transmembrane domain.

An internal linker in a first anti-FLT3 antigen binding domain can form a loop structure and a second antigen binding domain can be proximal to the transmembrane domain.

An internal linker in a second antigen binding domain can form a loop structure and a first anti-FLT3 antigen binding domain is proximal to the transmembrane domain.

A transmembrane domain can comprise a CD8a or CD28 transmembrane domain or a transmembrane fragment thereof. A CD8a transmembrane domain can comprise the amino acid sequence of SEQ ID NO: 71. A CD28 transmembrane domain can comprise the amino acid sequence of SEQ ID NO: 79.

An intracellular domain can comprise a 4-1BB costimulatory domain comprising the amino acid sequence of SEQ ID NO: 73 or a CD28 costimulatory domain comprising the amino acid sequence of SEQ ID NO: 81, and a CD3ζ intracellular T-cell signaling sequence comprising the amino acid sequence of SEQ ID NO: 74.

An intracellular T-cell signaling domain can comprise any number and any combination of costimulatory domains. Costimulatory domains include, but not limited to, 4-1BB costimulatory domains, CD28 costimulatory domains, ICOS stimulatory domains, OX-40 costimulatory domains, CD40L costimulatory domains, CD27 costimulatory domains and TLR2 costimulatory domains. Sequences of the preceding costimulatory domains are known in the art. When more than one costimulatory domain is present in a single T-cell signaling domain, the costimulatory domains can be present in any order.

In some aspects, an intracellular T-cell signaling domain can comprise a CD28 costimulatory domain. In some aspects, an intracellular T-cell signaling domain can comprise a 4-1BB costimulatory domain. In some aspects, an intracellular T-cell signaling domain can comprise a CD28 costimulatory domain and a 4-1BB costimulatory domain. In some aspects, an intracellular T-cell signaling domain can comprise a CD28 costimulatory domain and an OX-40 costimulatory domain.

In some aspects, an intracellular T-cell signaling domain can comprise a CD3ζ intracellular T-cell signaling sequence. In some aspects, an intracellular T-cell signaling domain can comprise a CD3ζ intracellular T-cell signaling sequence and a CD28 costimulatory domain. In some aspects, an intracellular T-cell signaling domain can comprise a CD3ζ intracellular T-cell signaling sequence and a 4-1BB costimulatory domain. In some aspects, an intracellular T-cell signaling domain can comprise a CD3ζ intracellular T-cell signaling sequence, a CD28 costimulatory domain and a 4-1BB costimulatory domain. In some aspects, an intracellular T-cell signaling domain can comprise a CD3ζ intracellular T-cell signaling sequence, a CD28 costimulatory domain and an OX-40 costimulatory domain.

A bivalent CAR of the present disclosure can further comprise a nucleotide sequence encoding a hinge domain. A hinge domain can be a CD8 hinge domain comprising the amino acid sequence of SEQ ID NO: 72 or a CD28 hinge domain comprising the amino acid sequence of SEQ ID NO: 80.

The present disclosure provides an expression construct comprising a nucleotide sequence encoding a bivalent CAR of the present disclosure. The present disclosure provides a recombinant expression vector comprising an expression construct of the present disclosure. The present disclosure provides a bivalent CAR expressed from a recombinant expression vector of the present disclosure. The present disclosure provides a host cell comprising a recombinant expression vector of the present disclosure. A host cell can be a T-cell. The present disclosure provides a population of cells comprising at least one host cell of the present disclosure.

The present disclosure provides a method of treating acute lymphoblastic leukemia (ALL) or acute myeloid leukemia (AML) in a subject, the method comprising administering to the subject an effective amount of the host cells of the present disclosure. An effective amount of host cells can comprise at least 1×10⁶ cells. ALL can be B-cell ALL (B-ALL).

The present disclosure provides a bivalent CAR of the present disclosure for use in the treatment of acute lymphoblastic leukemia (ALL) or acute myeloid leukemia (AML). In some aspects, treatment can comprise administering to the subject an effective amount of the T-cells, optionally wherein the effective amount comprises at least 1×10⁶ cells.

A bivalent CAR of the present disclosure can be expressed on the surface of a T-cell.

The present disclosure provides a pair of chimeric antigen receptors (CAR) comprising: a first CAR comprising: an anti-FLT3 antigen binding domain; a transmembrane domain; and an intracellular T-cell signaling domain; and a second CAR comprising: an antigen binding domain selected from the group consisting of an anti-CD19 antigen binding domain; an anti-CD22 antigen binding domain; an anti-CD33 antigen binding domain and an anti-CD123 antigen binding domain; a transmembrane domain; and an intracellular T-cell signaling domain.

An anti-FLT3 antigen binding domain of a first CAR can comprise: an anti-FLT3 single chain variable fragment (scFv) NC7 variable heavy chain fragment (SEQ ID NO: 16) and variable light chain fragment (SEQ ID NO: 17); an anti-FLT3 single chain variable fragment (scFv) m1006 variable heavy chain fragment (SEQ ID NO: 82) and variable light chain fragment (SEQ ID NO: 83); or an anti-FLT3 single chain variable fragment (scFv) m1007 variable heavy chain fragment (SEQ ID NO: 85) and variable light chain fragment (SEQ ID NO: 86).

A second CAR can comprise an anti-CD19 antigen binding domain comprising anti-CD19 scFv FMC63 variable heavy chain fragment (SEQ ID NO: 33) and variable light chain fragment (SEQ ID NO: 34).

A second CAR can comprise an anti-CD22 antigen binding domain comprising anti-CD22 scFV m971 variable heavy chain fragment (SEQ ID NO: 49) and variable light chain fragment (SEQ ID NO: 50).

A second CAR can comprise: an anti-CD33 antigen binding domain comprising anti-CD33 HuM195 variable heavy chain fragment (SEQ ID NO: 51) and variable light chain fragment (SEQ ID NO: 52); or an anti-CD33 scFc h-p67.6 variable heavy chain fragment (SEQ ID NO: 55) and variable light chain fragment (SEQ ID NO: 56).

A second CAR can comprise: an anti-CD123 scFv 26292 variable heavy chain fragment (SEQ ID NO: 88) and variable light chain fragment (SEQ ID NO: 92); an anti-CD123 scFv 32701 variable heavy chain fragment (SEQ ID NO: 96) and variable light chain fragment (SEQ ID NO: 100); an anti-CD123 scFv 32716 variable heavy chain fragment (SEQ ID NO: 104) and variable light chain fragment (SEQ ID NO: 108); or an anti-CD123 scFv 32703 variable heavy chain fragment (SEQ ID NO: 112) and variable light chain fragment (SEQ ID NO: 116).

A first CAR and a second CAR can each further comprise a CD8a or CD28 transmembrane domain or a transmembrane fragment thereof. The CD8a transmembrane domain can comprise the amino acid sequence of SEQ ID NO: 71 and the CD28 transmembrane domain can comprise the amino acid sequence of SEQ ID NO: 79.

An intracellular T-cell signaling domain of each of a first CAR and a second CAR can comprise a 4-1BB costimulatory domain comprising the amino acid sequence of SEQ ID NO: 73 or a CD28 costimulatory domain comprising the amino acid sequence of SEQ ID NO: 81, and a CD3ζ intracellular T-cell signaling sequence comprising the amino acid sequence of SEQ ID NO: 74.

An intracellular T-cell signaling domain of each of a first CAR and a second CAR can comprise any number and any combination of costimulatory domains. Costimulatory domains include, but not limited to, 4-1BB costimulatory domains, CD28 costimulatory domains, ICOS stimulatory domains, OX-40 costimulatory domains, CD40L costimulatory domains, CD27 costimulatory domains and TLR2 costimulatory domains. Sequences of the preceding costimulatory domains are known in the art. When more than one costimulatory domain is present in a single T-cell signaling domain, the costimulatory domains can be present in any order.

In some aspects, an intracellular T-cell signaling domain of each of a first CAR and a second CAR can comprise a CD28 costimulatory domain. In some aspects, An intracellular T-cell signaling domain of each of a first CAR and a second CAR can comprise a 4-1BB costimulatory domain. In some aspects, an intracellular T-cell signaling domain of each of a first CAR and a second CAR can comprise a CD28 costimulatory domain and a 4-1BB costimulatory domain. In some aspects, An intracellular T-cell signaling domain of each of a first CAR and a second CAR can comprise a CD28 costimulatory domain and an OX-40 costimulatory domain.

In some aspects, an intracellular T-cell signaling domain of each of a first CAR and a second CAR can comprise a CD3ζ intracellular T-cell signaling sequence. In some aspects, An intracellular T-cell signaling domain of each of a first CAR and a second CAR can comprise a CD3ζ intracellular T-cell signaling sequence and a CD28 costimulatory domain. In some aspects, an intracellular T-cell signaling domain of each of a first CAR and a second CAR can comprise a CD3ζ intracellular T-cell signaling sequence and a 4-1BB costimulatory domain. In some aspects, an intracellular T-cell signaling domain of each of a first CAR and a second CAR can comprise a CD3ζ intracellular T-cell signaling sequence, a CD28 costimulatory domain and a 4-1BB costimulatory domain. In some aspects, an intracellular T-cell signaling domain of each of a first CAR and a second CAR can comprise a CD3ζ intracellular T-cell signaling sequence, a CD28 costimulatory domain and an OX-40 costimulatory domain.

A first CAR and a second CAR can further comprise a hinge domain. A hinge domain can be a CD8 hinge domain comprising the amino acid sequence of SEQ ID NO: 72 or a CD28 hinge domain comprising the amino acid sequence of SEQ ID NO: 80.

The present disclosure provides an expression construct comprising a nucleotide sequence encoding the pair of CARs of the present disclosure, wherein a nucleotide sequence encoding the first CAR is joined to a nucleotide sequence encoding the second CAR by a nucleotide that encodes a self-cleaving linker peptide. The present disclosure provides a recombinant expression vector comprising an expression construct of the present disclosure.

The present disclosure provides a host cell comprising a pair of CARs of the present disclosure and/or a recombinant expression vector of the present disclosure. A host cell can be a T-cell. The present disclosure provides a population of cells comprising at least one host cell of the present disclosure.

The present disclosure provides a method of treating acute lymphoblastic leukemia (ALL) or acute myeloid leukemia (AML) in a subject, the method comprising administering to the subject an effective amount of the host cells of the present disclosure. An effective amount of host cells can comprise at least 1×10⁶ cells. ALL can be B-cell ALL (B-ALL).

The present disclosure provides a pair of CARs of any one of claims 29-39 for use in the treatment of acute lymphoblastic leukemia (ALL) or acute myeloid leukemia (AML).

A pair of CARs of the present disclosure can be expressed on the surface of a T-cell.

The present disclosure provides a bicistronic chimeric antigen receptor (CAR) construct comprising: i) a first group of nucleotide sequences comprising: a) a nucleotide sequence encoding a first anti-FLT3 antigen binding domain; b) a nucleotide sequence encoding a first transmembrane domain; and c) a nucleotide sequence encoding a first intracellular T-cell signaling domain; ii) a second group of nucleotide sequences comprising: a) a nucleotide sequence encoding an antigen binding domain selected from the group consisting of an anti-CD19 antigen binding domain, an anti-CD22 antigen binding domain, an anti-CD33 antigen binding domain and an anti-CD123 antigen binding domain; b) a nucleotide sequence encoding a second transmembrane domain; and c) a nucleotide sequence encoding a second intracellular T-cell signaling domain; and iii) a cleavable linker nucleotide sequence between the first group of nucleotide sequences and the second group of nucleotide sequences encoding a cleavable linker peptide.

A nucleotide sequence encoding a first anti-FLT3 antigen binding domain can encode a variable heavy chain fragment (SEQ ID NO: 16) and a variable light chain fragment (SEQ ID NO: 17) of a single chain variable fragment (scFv) of anti-FLT3 NC7; a variable heavy chain fragment (SEQ ID NO: 82) and a variable light chain fragment (SEQ ID NO: 83) of a scFv of anti-FLT3 m1006, or a variable heavy chain fragment (SEQ ID NO: 85) and a variable light chain fragment (SEQ ID NO: 85) of a scFv of anti-FLT3 m1007.

A nucleotide sequence encoding a second antigen binding domain can encode a variable heavy chain fragment (SEQ ID NO: 33) and variable light chain fragment (SEQ ID NO: 34) of a single chain variable fragment (scFv) of anti-CD19 FMC63.

A nucleotide sequence encoding a second antigen binding domain can encode a variable heavy chain fragment (SEQ ID NO: 49) and variable light chain fragment (SEQ ID NO: 50) of a single chain variable fragment (scFv) of anti-CD22 m971.

A nucleotide sequence encoding a second antigen binding domain can encode a variable heavy chain fragment (SEQ ID NO: 51) and a variable light chain fragment (SEQ ID NO: 52) of a single chain variable fragment (scFv) of anti-CD33 HuM195, or a variable heavy chain fragment (SEQ ID NO: 55) and a variable light chain fragment (SEQ ID NO: 56) of a single chain variable fragment (scFv) of anti-CD33 h-p67.6.

A nucleotide sequence encoding a second antigen binding domain encodes: a variable heavy chain fragment (SEQ ID NO: 88) and variable light chain fragment (SEQ ID NO: 92) of anti-CD123 scFv 26292; a variable heavy chain fragment (SEQ ID NO: 96) and variable light chain fragment (SEQ ID NO: 100) of anti-CD123 scFv 32701; a heavy chain fragment (SEQ ID NO: 104) and variable light chain fragment (SEQ ID NO: 108) of anti-CD123 scFv 32716; or a heavy chain fragment (SEQ ID NO: 112) and variable light chain fragment (SEQ ID NO: 116) of anti-CD123 scFv 32703.

A first and second transmembrane domains can comprise a CD8 transmembrane domain comprising the amino acid sequence of SEQ ID NO: 71 or a CD28 transmembrane domain comprising the amino acid sequence of SEQ ID NO: 79.

A first and second intracellular T-cell signaling domains can comprise a 4-1BB costimulatory domain comprising the amino acid sequence of SEQ ID NO: 73 or a CD28 costimulatory domain comprising the amino acid sequence of SEQ ID NO: 81, and a CD3ζ intracellular T-cell signaling sequence comprising the amino acid sequence of SEQ ID NO: 74.

A first and second intracellular T-cell signaling domain can comprise any number and any combination of costimulatory domains. Costimulatory domains include, but not limited to, 4-1BB costimulatory domains, CD28 costimulatory domains, ICOS stimulatory domains, OX-40 costimulatory domains, CD40L costimulatory domains, CD27 costimulatory domains and TLR2 costimulatory domains. Sequences of the preceding costimulatory domains are known in the art. When more than one costimulatory domain is present in a single T-cell signaling domain, the costimulatory domains can be present in any order.

In some aspects, a first and second intracellular T-cell signaling domain can comprise a CD28 costimulatory domain. In some aspects, a first and second intracellular T-cell signaling domain can comprise a 4-1BB costimulatory domain. In some aspects, a first and second intracellular T-cell signaling domain can comprise a CD28 costimulatory domain and a 4-1BB costimulatory domain. In some aspects, a first and second intracellular T-cell signaling domain can comprise a CD28 costimulatory domain and an OX-40 costimulatory domain.

In some aspects, a first and second intracellular T-cell signaling domain can comprise a CD3ζ intracellular T-cell signaling sequence. In some aspects, a first and second intracellular T-cell signaling domain can comprise a CD3ζ intracellular T-cell signaling sequence and a CD28 costimulatory domain. In some aspects, a first and second intracellular T-cell signaling domain can comprise a CD3ζ intracellular T-cell signaling sequence and a 4-1BB costimulatory domain. In some aspects, a first and second intracellular T-cell signaling domain can comprise a CD3ζ intracellular T-cell signaling sequence, a CD28 costimulatory domain and a 4-1BB costimulatory domain. In some aspects, a first and second intracellular T-cell signaling domain can comprise a CD3ζ intracellular T-cell signaling sequence, a CD28 costimulatory domain and an OX-40 costimulatory domain.

A first group of nucleotide sequences and a second group of nucleotide sequences can each further comprise a nucleotide sequence encoding a hinge domain, wherein the hinge domain is a CD8 hinge domain comprising the amino acid sequence of SEQ ID NO: 72 or a CD28 hinge domain comprising the amino acid sequence of SEQ ID NO: 80.

A self-cleaving linker peptide can be a 2A linker selected from the group consisting of T2A (EGRGSLLTCGDVEENPGP; SEQ ID NO: 75), P2A (ATNFSLLKQAGDVEENPGP; SEQ ID NO: 76), E2A (QCTNYALLKLAGDVESNPGP; SEQ ID NO: 77), F2A (VKQTLNFDLLKLAGDVESNPGP; SEQ ID NO: 78), or a furin-cleavable linker. A self-cleaving linker peptide can be a GSG-2A linker selected from the group consisting of GSG-T2A (SEQ ID NO: 57), GSG-P2A (SEQ ID NO: 58), GSG-E2A (SEQ ID NO: 59) and GSG-F2A (SEQ ID NO: 60).

The present disclosure provides a recombinant expression vector comprising a bicistronic CAR construct of the present disclosure. The present disclosure provides a host cell comprising a recombinant expression vector of the present disclosure. A host cell can be a T-cell. The present disclosure provides a population of cells comprising at least one host cell of the present disclosure.

In various aspects, the present disclosure provides multispecific chimeric antigen receptors (CARs) having antigenic specificity fms-like tyrosine kinase 3 (FLT3) and one of CD19, CD22, CD33 and CD123. In some embodiments, the multispecific CAR is a bivalent CAR comprising an anti-FLT3 antigen binding domain; one of an anti-CD19 antigen binding domain, an anti-CD22 antigen binding domain, an anti-CD33 antigen binding domain or an anti-CD123 antigen binding domain; a transmembrane domain; and an intracellular domain. In other embodiments, the multispecific CAR is a bicistronic CAR comprising a first CAR comprising an anti-FLT3 antigen binding domain, a transmembrane domain, and an intracellular T-cell signaling domain; and a second CAR comprising an antigen binding domain selected from the group consisting of an anti-CD19 antigen binding domain; an anti-CD22 antigen binding domain; an anti-CD33 antigen binding domain; and an anti-CD123 antigen binding domain; a transmembrane domain, and an intracellular T-cell signaling domain. The two CARs of the bicistronic CAR are expressed together from a common expression construct in a host cell.

In one aspect, bivalent CARs are provided comprising: a first anti-FLT3 antigen binding domain; a second antigen binding domain selected from the group consisting of an anti-CD19 antigen binding domain, an anti-CD22 antigen binding domain, an anti-CD33 antigen binding domain and an anti-CD123 antigen binding domain; at least one linker domain; a transmembrane domain; and an intracellular T-cell signaling domain.

In another aspect, a pair of CARs is provided, comprising a first CAR comprising an anti-FLT3 antigen binding domain, a transmembrane domain, and an intracellular T-cell signaling domain; and a second CAR comprising an antigen binding domain selected from the group consisting of an anti-CD19 antigen binding domain; an anti-CD22 antigen binding domain; an anti-CD33 antigen binding domain; and an anti-CD123 antigen binding domain, a transmembrane domain, and an intracellular T-cell signaling domain.

In another aspect, a bicistronic CAR construct is provided, comprising,
i) a first group of nucleotide sequences comprising:
   a) a nucleotide sequence encoding a first anti-FLT3 antigen binding domain;
   b) a nucleotide sequence encoding a first transmembrane domain; and
   c) a nucleotide sequence encoding a first intracellular T-cell signaling domain;
ii) a second group of nucleotide sequences comprising:
   d) a nucleotide sequence encoding an antigen binding domain selected from the group consisting of an anti-CD19 antigen binding domain; an anti-CD22 antigen binding domain; an anti-CD33 antigen binding domain; and an anti-CD123 antigen binding domain;
   e) a nucleotide sequence encoding a second transmembrane domain; and
   f) a nucleotide sequence encoding a second intracellular T-cell signaling domain; and
iii) a cleavable linker nucleotide sequence between the first group of nucleotide sequences and the second group of nucleotide sequences encoding a cleavable linker peptide.

The CARs and CAR constructs disclosed herein can be expressed via an expression vector in a host cell, such as T-cells.

Methods for treating acute lymphoblastic leukemia (ALL) or acute myeloid leukemia (AML) in a subject using the CARs and CAR constructs described herein, and host cells expressing these are also provided.

Use of the CARs and CAR constructs described herein, and host cells expressing these in the treatment of ALL or AML is provided.

Use of the CARs and CAR constructs described herein, and host cells expressing these in the manufacture of a medicament for use in treatment of ALL or AML is provided.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more elements. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1A is a schematic illustrating a tandem bivalent chimeric antigen receptor (CAR) according to an embodiment of the present disclosure, wherein the anti-FLT3 antigen binding domain 102 is distal to the transmembrane domain 120.

FIG. 1B is a schematic illustrating a tandem bivalent CAR according to an embodiment of the present disclosure, wherein the anti-FLT3 antigen binding domain 102 is proximal to the transmembrane domain 120.

FIG. 2A is a schematic illustrating a loop bivalent CAR according to an embodiment of the present disclosure, wherein the anti-FLT3 antigen binding domain 102 is distal to the transmembrane domain 120.

FIG. 2B is a schematic illustrating a loop bivalent CAR according to an embodiment of the present disclosure, wherein the anti-FLT3 antigen binding domain 102 is proximal to the transmembrane domain 120.

FIG. 3 is a schematic illustrating an expression construct that encodes a tandem bivalent CAR according to an embodiment of the present disclosure, wherein the anti-FLT3 antigen binding domain 102 is distal to the transmembrane domain 120 when expressed.

FIG. 4 is a schematic illustrating an expression construct that encodes a loop bivalent CAR according to an embodiment of the present disclosure, wherein the anti-FLT3 antigen binding domain 102 is proximal to the transmembrane domain 120 when expressed.

FIG. 5 is a schematic illustrating a bicistronic expression construct encoding two distinct CARs according to an embodiment of the present disclosure.

FIG. 6 is a series of plots from Fluorescence-activated cell sorting (FACS) cell analysis of T-cells transfected with expression vectors encoding for bicistronic CAR constructs of the present disclosure.

FIG. 7 is a graph showing IL-2 secretion by T-cells expressing bicistronic CAR constructs of the present disclosure incubated with various cancer cell lines.

FIG. 8 is a graph showing IFN-γ secretion by T-cells expressing bicistronic CAR constructs of the present disclosure incubated with various cancer cell lines.

FIG. 9 is a graph showing tumor growth in mice infected with SEM LUC+ cells and treated with T-cells expressing bicistronic CAR constructs of the present disclosure.

FIG. 10 is a graph showing the body weight of mice infected with SEM LUC+ cells and treated with T-cells expressing bicistronic CAR constructs of the present disclosure.

FIG. 11 is a series of images showing tumor growth in mice infected with MOLM14 LUC+ cells and treated with T-cells expressing bicistronic CAR constructs of the present disclosure.

### DETAILED DESCRIPTION

In the following sections, various compositions and methods are described in order to detail various embodiments. Practicing the various embodiments does not require the employment of all of the specific details outlined herein, but rather concentrations, time, and other specific details may be modified. In some cases, well known methods or components have not been included in the description.

Embodiments of the present disclosure provide multispecific chimeric antigen receptors (CARs) having antigenic specificity fms-like tyrosine kinase 3 (FLT3) and one of CD19, CD22, CD33, or CD123. In various aspects, the multispecific CAR is a bivalent CAR comprising an anti-FLT3 antigen binding domain; one of an anti-CD19 antigen binding domain, an anti-CD22 antigen binding domain, an anti-CD33 antigen binding domain or an anti-CD123 antigen binding domain; a transmembrane domain; and an intracellular domain.

In other embodiments, the multispecific CAR is a bicistronic CAR comprising a first CAR comprising an anti-FLT3 antigen binding domain, a transmembrane domain, and an intracellular T-cell signaling domain; and a second CAR comprising an antigen binding domain selected from the group consisting of an anti-CD19 antigen binding domain; an anti-CD22 antigen binding domain; an anti-CD33 antigen binding domain; and an anti-CD123 antigen binding domain, a transmembrane domain, and an intracellular T-cell signaling domain. The two CARs of the bicistronic CAR are expressed together from a common expression construct in a host cell.

A CAR is an artificially constructed hybrid protein or polypeptide that generally includes an antigen binding domain of an antibody (e.g., a single chain variable fragment (scFv)) linked to a T-cell transmembrane domain and a T-cell signaling domain. CARs are able to redirect T-cell specificity and reactivity toward a selected target in a non-MHC (major histocompatibility complex)-restricted manner, exploiting the antigen-binding properties of monoclonal antibodies. The non-MHC-restricted antigen recognition gives T-cells expressing CARs the ability to recognize antigens independent of antigen processing, thus bypassing a major mechanism of tumor escape. Moreover, when expressed in T-cells, CARs advantageously do not dimerize with endogenous T-cell receptor (TCR) alpha and beta chains.

FLT3, also known as Fins-Related Tyrosine Kinase 3, Stem Cell Tyrosine Kinase 1, FL Cytokine Receptor, CD135, FLK-2, and STK-1, is frequently mutated in acute myeloid leukemia (ALL). This causes activation of the disease pathway and is thought to be a major driver of disease. Thus, down-regulation of FLT3 is an improbable escape mechanism. FLT3 mutations are found in the intracellular domain of the receptor, so immune cells expressing the FLT3 CARs described herein are able to target both wild type and mutant forms of FLT3, allowing for broad targeting of both ALL and AML, as well as other FLT3-expressing leukemias.

CD19, also known as B-cell lymphocyte antigen CD19, B4, and CVID3, is a cell surface molecule expressed only by B lymphocytes and follicular dendritic cells of the hematopoietic system. CART cells targeting CD19 have demonstrated potent efficacy in children and young adults with relapsed and chemotherapy refractory B cell acute lymphoblastic leukemia (B-ALL). Although complete remission rates approach 65%-80%, not all patients respond to CD19 CART therapy, and relapse due to loss of CD19 can occur in over one third of cases. CD19 antigen loss has similarly been observed in treatment of adult lymphoma.

CD22 is a lineage-restricted B cell antigen belonging to the immunoglobulin (lg) superfamily and is expressed in 60%-70% of all B cell lymphomas and leukemias (e.g., B-chronic lymphocytic leukemia, hairy cell leukemia, acute lymphocytic leukemia (ALL), and Burkitt's lymphoma). The antigen is not present on the cell surface in early stages of B cell development or on stem cells. CD22 CART cells have demonstrated a similar degree of clinical activity to CD19 CART cells. Relapse due to CD22 loss or diminution of surface expression has been observed.

CD33, also known as SIGLEC3, Gp67, is a membrane spanning Ig-like receptor that plays a role in myeloid differentiation and dendritic cell maturation. The antigen is overexpressed on leukemic blast cells and myeloid leukemia initiating cells and CD33 ligation by a monoclonal antibody has been demonstrated to inhibit AML proliferation. Absent from primitive stem cells and multipotent progenitor cells, CD33 levels on mature granulocytes and circulating macrophages are low, making it an attractive immunotherapy target in AML.

CD123, also known as interleukin-3 receptor, IL-3RA is a molecule found on cells which helps transmit interleukin-3 signals. CD123 belongs to the type I cytokine receptor family and comprises a heterodimer of a unique alpha chain paired with the common beta (beta c or CD131) subunit. The receptor is found on pluripotent progenitor cells and promotes proliferation and differentiation with the hematopoietic cell line. CD123 has been found to be expressed across acute myeloid leukemia (AML) subtypes, including leukemic stem cells.

Bivalent Chimeric Antigen Receptors

Various inventive aspects described herein provide CARs having two binding domains on the same CAR, which will be referred to herein as a “bivalent” CAR. Bivalent CARs of the present disclosure comprise a first anti-FLT3 antigen binding domain that is specific for FLT3, a second antigen binding domain that is specific for CD19, CD22, CD33 or CD123 at least one inter-binding domain linker, a transmembrane domain, and an intracellular T-cell signaling domain.

In some embodiments, the bivalent CAR is a FLT3×CD19 CAR. In some embodiments, the bivalent CAR is a FLT3×CD22 CAR. In some embodiments, the bivalent CAR is a FLT3×CD33 CAR. In some embodiments, the bivalent CAR is a FLT3×CD123 CAR.

The bivalent CARs of the present disclosure provide significant advantages over a univalent CAR having a single antigen-specific binding domain. For example, the bivalent CARS advantageously provides greater potency, and reduces or prevents cell escape due to loss or reduction in the expression of one of FLT3, CD19, CD22, CD33 or CD123. Bivalent CARs comprising FLT3- and CD19-specific antigen binding domains, or FLT3 and CD22-specific binding domains, can be used to treat ALL as well as treat relapsed or refractory ALL in which CD19 or CD22 surface protein expression is lost or significantly reduced. Similarly, bivalent CARs comprising FLT3- and CD33-specific antigen binding domains or FLT3- and CD123-specific antigen binding domains can be used to treat AML as well as treat relapsed or refractory AML in which surface protein expression of one antigen (e.g. CD33 or CD123 or FLT3) is lost or significantly reduced. Bivalent CARs comprising FLT3- and CD123-specific antigen binding domains can also be used to treat precursor B-cell ALL.

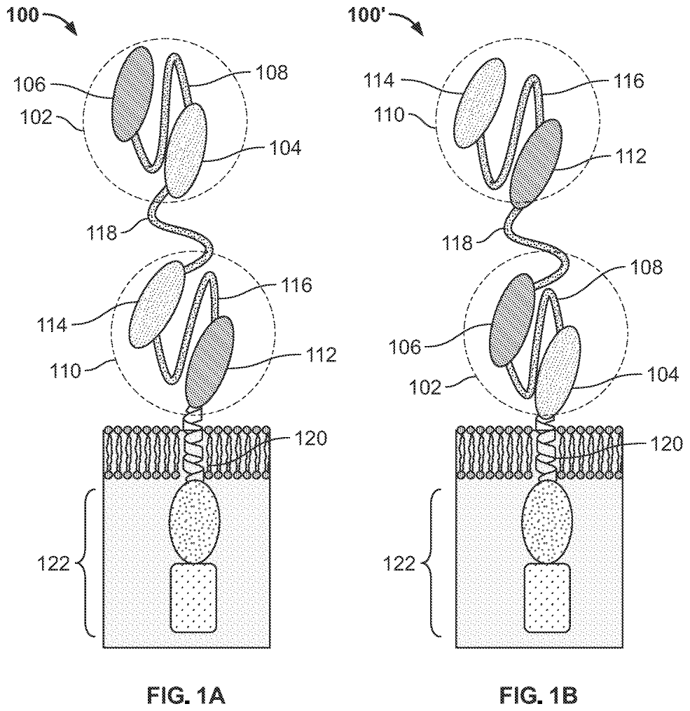

FIG. 1A is a schematic of a representative bivalent CAR 100 according to an embodiment of the present disclosure. Bivalent CAR 100 comprises: a first anti-FLT3 antigen binding domain 102; a second antigen binding domain 110 selected from the group of: an anti-CD19 antigen binding domain, an anti-CD22 antigen binding domain, an anti-CD33 antigen binding domain and an anti-CD123 antigen binding domain; an inter-binding domain linker 118; a transmembrane domain 120; and an intracellular T-cell signaling domain 122. As presented in FIG. 1A, the first anti-FLT3 antigen binding domain 102 is distal to the transmembrane domain 120.

FIG. 1B is a schematic of a representative bivalent CAR 100′ according to an embodiment of the present disclosure. Bivalent CAR 100′ comprises: a first anti-FLT3 antigen binding domain 102; a second antigen binding domain 110 selected from the group of an anti-CD19 antigen binding domain, an anti-CD22 antigen binding domain, an anti-CD33 antigen binding domain and an anti-CD123 antigen binding domain; an inter-binding domain linker 118; a transmembrane domain 120; and an intracellular T-cell signaling domain 122. As presented in FIG. TA, the first anti-FLT3 antigen binding domain 102 is proximal to the transmembrane domain 120.

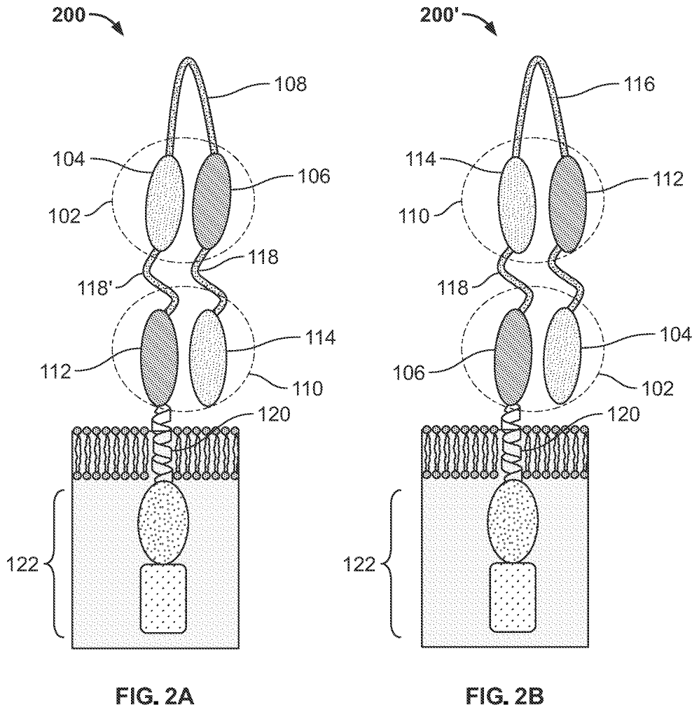

FIG. 2A is a schematic of a representative bivalent CAR 200 according to an embodiment of the present disclosure. Bivalent CAR 200 comprises: a first anti-FLT3 antigen binding domain 102; a second antigen binding domain 110 selected from the group of: an anti-CD19 antigen binding domain, an anti-CD22 antigen binding domain; an anti-CD33 antigen binding domain and an anti-CD123 antigen binding domain; two inter-binding domain linkers 118; a transmembrane domain 120; and an intracellular T-cell signaling domain 122. As presented in FIG. 2A, the first anti-FLT3 antigen binding domain 102 is distal to the transmembrane domain 120.

FIG. 2B is a schematic of a representative bivalent CAR 200′ according to an embodiment of the present disclosure. Bivalent CAR 200 comprises a first anti-FLT3 antigen binding domain 102, a second antigen binding domain 110 selected from the group of an anti-CD19 antigen binding domain, an anti-CD22 antigen binding domain, an anti-CD33 antigen binding domain and an anti-CD123 antigen binding domain; two inter-binding domain linkers 118, a transmembrane domain 120, and an intracellular T-cell signaling domain 122. As presented in FIG. 2B, the first anti-FLT3 antigen binding domain 102 is proximal to the transmembrane domain 120.

The first anti-FLT3 antigen binding domain 102 depicted in FIGS. TA, 1B, and 2A comprises a variable light chain (V_{L}) 104, a variable heavy chain (V_{H}) 106, and an intra-binding domain linker 108. The V_{H} and V_{L} of the first anti-FLT3 antigen binding domain 102 may be derived from a single chain variable fragment (scFv), which is itself derived from a portion of an antibody that specifically recognizes an epitope of FLT3. The scFv (and V_{H} and V_{L} thereof) of the first anti-FLT3 antigen binding domain 102 can be derived from any specific FLT3 antibody. In certain embodiments, the scFv of the first anti-FLT3 antigen binding domain 102 is derived from an FLT3 antibody m1006 or m1007. In certain embodiments, the scFv of the first anti-FLT3 antigen binding domain 102 is derived from the FLT3 antibody NC7. The antigen binding domain of NC7 specifically binds to FLT3. In some embodiments, the first anti-FLT3 antigen binding domain 102 comprises, consists of, or consists essentially of the V_{H} and V_{L} of NC7 scFv. In one embodiment, the first anti-FLT3 antigen binding domain 102 is formed by the V_{H} and V_{L} of NC7 scFv.

NC7 is described in, for example, U.S. Pat. No. 8,071,099. The NC7 scFv comprises a V_{L} and a V_{H}. In certain embodiments, the NC7 scFv V_{H} comprises a CDR1 region, a CDR2 region, and a CDR3 region. In this regard, the NC7 scFv V_{H} comprises one or more of a V_{H} CDR1 region having the amino acid sequence of SEQ ID NO: 2, a V_{H} CDR2 region comprising the amino acid sequence of SEQ ID NO: 4, and a V_{H} CDR3 region comprising the amino acid sequence of SEQ ID NO: 6. In some embodiments, NC7 scFv V_{H} comprises each amino acid sequence of SEQ ID NOs: 2, 4, and 6.

In certain embodiments, the NC7 scFv V_{L} comprises a CDR1 region, a CDR2 region, and a CDR3 region. In this regard, the NC7 scFv V_{L} comprises one or more of a V_{L} CDR1 region having the amino acid sequence of SEQ ID NO: 10, a V_{L} CDR2 region comprising the amino acid sequence of SEQ ID NO: 12, and a V_{L} CDR3 region comprising the amino acid sequence of SEQ ID NO: 14. In some embodiments, NC7 scFv V_{L} comprises each amino acid sequence of SEQ ID NOs: 10, 12, and 14.

In an embodiment, the first anti-FLT3 antigen binding domain 102 comprises each amino acid sequence of SEQ ID NOs: 2, 4, 6, 10, 12, and 14.

The NC7 scFv V_{H} may comprise, consist of, or consist essentially of the amino acid sequence of SEQ ID NO: 16. In particular embodiments, the NC7 scFv V_{H} is SEQ ID NO: 16. The NC7 scFv V_{L} may comprise, consist of, or consist essentially of the amino acid sequence of SEQ ID NO: 17. In particular embodiments, the NC7 scFv V_{L} is SEQ ID NO: 17. Accordingly, in an embodiment, the first anti-FLT3 antigen binding domain comprises a V_{H} comprising the amino acid sequence of SEQ ID NO: 16 and/or a V_{L} comprising the amino acid sequence of SEQ ID NO: 17. In an embodiment, the first anti-FLT3 antigen binding domain comprises amino acid sequences of both SEQ ID NO: 16 and 17.

The amino acid sequences for full-length NC7 scFv and its components (V_{L} 104, V_{H} 106, and intra-binding domain linker 108) are provided in Table 1.

TABLE 1
Amino acid sequences for FLT3-specific NC7 scFv

| Description | Sequence | SEQ ID NO: |
|---|---|---|
| V_{H}-FR1 | EVQLVQSGAEVKKPGSSVKVSCKAS | 1 |
| V_{H}-CDR1 | GGTFSSYAIS | 2 |
| V_{H}-FR2 | WVRQAPGQGLEWMG | 3 |
| V_{H}-CDR2 | GIIPIFGTANYAQKFQG | 4 |
| V_{H}-FR3 | RVTITADKSTSTAYMELSSLRSEDTAVYYCAT | 5 |
| V_{H}-CDR3 | FALFGFREQAFDI | 6 |
| V_{H}-FR4 | WGQGTTVTVSS | 7 |
| Linker | GGGGSGGGGSGGGGS | 8 |
| V_{L}-FR1 | DIQMTQSPSSLSASVGDRVTITC | 9 |
| V_{L}-CDR1 | RASQSISSYLN | 10 |
| V_{L}-FR2 | WYQQKPGKAPKLLIY | 11 |
| V_{L}-CDR2 | AASSLQS | 12 |
| V_{L}-FR3 | GVPSRFSGSGSGTDFTLTISSLQPEDLATYYC | 13 |
| V_{L}-CDR3 | QQSYSTPFT | 14 |
| V_{L}-FR4 | FGPGTKVDIK | 15 |
| V_{H} | EVQLVQSGAEVKKPGSSVKVSCKASGGTFSSYAISWVRQA | 16 |
| | PGQGLEWMGGIIPIFGTANYAQKFQGRVTITADKSTSTAYM | |
| | ELSSLRSEDTAVYYCATFALFGFREQAFDIWGQGTTVTVSS | |
| V_{L} | DIQMTQSPSSLSASVGDRVTITCRASQSISSYLNWYQQKPG | 17 |
| | KAPKLLIYAASSLQSGVPSRFSGSGSGTDFTLTISSLQPEDLA | |
| | TYYCQQSYSTPFTFGPGTKVDIK | |
| scFv-NC7 | EVQLVQSGAEVKKPGSSVKVSCKASGGTFSSYAISWVRQA | 18 |
| | PGQGLEWMGGIIPIFGTANYAQKFQGRVTITADKSTSTAYM | |
| | ELSSLRSEDTAVYYCATFALFGFREQAFDIWGQGTTVTVSS | |
| | GGGGSGGGGSGGGGSDIQMTQSPSSLSASVGDRVTITCRAS | |
| | QSISSYLNWYQQKPGKAPKLLIYAASSLQSGVPSRFSGSGS | |
| | GTDFTLTISSLQPEDLATYYCQQSYSTPFTFGPGTKVDIK | |

In certain embodiments, the scFv of the first anti-FLT3 antigen binding domain 102 is derived from FLT3 antibody M1006 or M1007. The antigen binding domains of M1006 and M1007 specifically bind to FLT3. In some embodiments, the first anti-FLT3 antigen binding domain 102 comprises, consists of, or consists essentially of the V_{H} and V_{L} of M1006 scFv. In one embodiment, the first anti-FLT3 antigen binding domain 102 is formed by the V_{H} and V_{L} of M1006 scFv. In some embodiments, the first anti-FLT3 antigen binding domain 102 comprises, consists of, or consists essentially of the V_{H} and V_{L} of M1007 scFv. In one embodiment, the first anti-FLT3 antigen binding domain 102 is formed by the V_{H} and V_{L} of M1007 scFv.

The M1006 scFv V_{H} may comprise, consist of, or consist essentially of the amino acid sequence of SEQ ID NO: 82. In particular embodiments, the M1006 scFv V_{H} is SEQ ID NO: 82. The M1006 scFv V_{L} may comprise, consist of, or consist essentially of the amino acid sequence of SEQ ID NO: 83. In particular embodiments, the M1006 scFv V_{L} is SEQ ID NO: 83. Accordingly, in an embodiment, the first anti-FLT3 antigen binding domain comprises a V_{H} comprising the amino acid sequence of SEQ ID NO: 82 and/or a V_{L} comprising the amino acid sequence of SEQ ID NO: 83. In an embodiment, the first anti-FLT3 antigen binding domain comprises amino acid sequences of both SEQ ID NO: 82 and 83. In an embodiment, the first anti-FLT3 antigen binding domain comprises SEQ ID NO: 84.

The M1007 scFv V_{H} may comprise, consist of, or consist essentially of the amino acid sequence of SEQ ID NO: 85. In particular embodiments, the M1006 scFv V_{H} is SEQ ID NO: 85. The M1007 scFv V_{L} may comprise, consist of, or consist essentially of the amino acid sequence of SEQ ID NO: 86. In particular embodiments, the M1007 scFv V_{L} is SEQ ID NO: 86. Accordingly, in an embodiment, the first anti-FLT3 antigen binding domain comprises a V_{H} comprising the amino acid sequence of SEQ ID NO: 85 and/or a V_{L} comprising the amino acid sequence of SEQ ID NO: 86. In an embodiment, the first anti-FLT3 antigen binding domain comprises amino acid sequences of both SEQ ID NO: 85 and 86. In an embodiment, the first anti-FLT3 antigen binding domain comprises SEQ ID NO: 87.

The first anti-FLT3 antigen binding domain 102 depicted in FIG. 2B comprises a V_{L} 104, and a V_{H} 106, but is missing an intra-binding domain linker. Whether the first anti-FLT3 antigen binding domain 102 comprises intra-binding domain linker 108 is dependent on the configuration of the bivalent CAR as provided by the present disclosure. As illustrated in FIG. 2B, V_{L} 102 and V_{H} 106 are each linked to one of V_{L} 114 or V_{H} 112 of the second antigen binding domain 110 via an inter-binding domain linker. 118.

In an embodiment, the first anti-FLT3 antigen binding domain 102 comprises a V_{H} having the amino acid sequence of SEQ ID NO: 16 and a V_{L} having the amino acid sequence of SEQ ID NO: 17.

The second antigen binding domain 110 depicted in FIGS. 1A, 1B, and 2B is selected from the group of an anti-CD19 antigen binding domain, an anti-CD22 antigen binding domain, an anti-CD33 antigen binding domain and an anti-CD123 antigen binding domain, and comprises a V_{L} 114, a V_{H} 112 of an antigen-specific scFv, and an intra-binding domain linker 116. The scFv of the second antigen domain 110 is derived from a portion of an antibody that specifically recognizes an epitope from one of CD19, CD22, CD33 or CD123. The scFv of the of the second antigen binding domain 110 can be derived from any specific antibody that binds CD19, CD22, CD33 or CD123.

In certain embodiments, the scFv of the second antigen binding domain 110 is derived from the CD19 antibody FMC63. The antigen binding domain of FMC63 specifically binds to CD19. In some embodiments, the second antigen binding domain 110 comprises, consists of, or consists essentially of the V_{H} and V_{L} of FMC63 scFv. In one embodiment, the second antigen binding domain 110 is formed by the V_{H} and V_{L} of FMC63 scFv.

The FMC63 scFv comprises a V_{L} and a V_{H}. In certain embodiments, the FMC63 scFv V_{H} comprises a CDR1 region, a CDR2 region, and a CDR3 region. In this regard, the FMC63 scFv V_{H} comprises one or more of a V_{H} CDR1 region having the amino acid sequence of SEQ ID NO: 20, a V_{H} CDR2 region comprising the amino acid sequence of SEQ ID NO: 22, and a V_{H} CDR3 region comprising the amino acid sequence of SEQ ID NO: 24. In some embodiments, FMC63 scFv V_{H} comprises each amino acid sequence of SEQ ID NOs: 20, 22, and 24.

In certain embodiments, the FMC63 scFv V_{L} comprises a CDR1 region, a CDR2 region, and a CDR3 region. In this regard, the FMC63 scFv V_{L} comprises one or more of a V_{L} CDR1 region having the amino acid sequence of SEQ ID NO: 27, a V_{L} CDR2 region comprising the amino acid sequence of SEQ ID NO: 29, and a V_{L} CDR3 region comprising the amino acid sequence of SEQ ID NO: 31. In some embodiments, FMC63 scFv V_{L} comprises each amino acid sequence of SEQ ID NOs: 27, 29, and 31.

In an embodiment, the second antigen binding domain is an anti-CD19 antigen binding domain and comprises each amino acid sequence of SEQ ID NOs: 20, 22, 24, 27, 29, and 31.

The FMC63 scFv V_{H} may comprise, consist of, or consist essentially of the amino acid sequence of SEQ ID NO: 33. The FMC63 scFv V_{L} may comprise, consist of, or consist essentially of the amino acid sequence of SEQ ID NO: 34. Accordingly, in an embodiment, second antigen binding domain comprises a V_{H} comprising the amino acid sequence of SEQ ID NO: 33 and/or a V_{L} comprising the amino acid sequence of SEQ ID NO: 34. In an embodiment, the second antigen binding domain comprises amino acid sequences of both SEQ ID NO: 33 and 34.

The amino acid sequences for FMC63 scFv and its components (V_{L} 104, V_{H} 106) are provided in Table 2.

TABLE 2
Amino acid sequences for CD19-specific FMC63 scFv

| Description | Sequence | SEQ ID NO: |
|---|---|---|
| V_{H}-FR1 | EVKLQESGPGLVAPSQSLSVTCTVS | 19 |
| V_{H}-CDR1 | GVSLPDYG | 20 |
| V_{H}-FR2 | VSWIRQPPRKGLEWLGV | 21 |
| V_{H}-CDR2 | IWGSETT | 22 |
| V_{H}-FR3 | YYNSALKSRLTIIKDNSKSQVFLKMNSLQTDDTAIYYC | 23 |
| V_{H}-CDR3 | AKHYYYGGSY AMDY | 24 |
| V_{H}-FR4 | WGQGTSVTVSS | 25 |
| V_{L}-FR1 | DIQMTQTTSSLSASLGDRVTISCRAS | 26 |
| V_{L}-CDR1 | QDISKY | 27 |
| V_{L}-FR2 | LNWYQQKPDGTVKLLIY | 28 |
| V_{L}-CDR2 | HTS | 29 |
| V_{L}-FR3 | RLHSGVPSRFSGSGSGTDYSLTISNLEQEDIATYFC | 30 |
| V_{L}-CDR3 | QQGNTLPYT | 31 |
| V_{L}-FR4 | FGGGTKLEIT | 32 |
| V_{H} | EVKLQESGPGLVAPSQSLSVTCTVSGVSLPDYGVSWIRQPP | 33 |
| | RKGLEWLGVIWGSETTYYNSALKSRLTIIKDNSKSQVFLKM | |
| | NSLQTDDTAIYYCAKHYYYGGSYAMDYWGQGTSVTVSS | |
| V_{L} | DIQMTQTTSSLSASLGDRVTISCRASQDISKYLNWYQQKPD | 34 |
| | GTVKLLIYHTSRLHSGVPSRFSGSGSGTDYSLTISNLEQEDI | |
| | ATYFCQQGNTLPYTFGGGTKLEIT | |

In certain embodiments, the scFv of the second antigen binding domain 110 is derived from the CD22 antibody m971. The antigen binding domain of m971 specifically binds to CD22. In some embodiments, the second antigen binding domain 110 comprises, consists of, or consists essentially of the V_{H} and V_{L} of m971 scFv. In one embodiment, the second antigen binding domain 110 is formed by the V_{H} and V_{L} of m971 scFv.

The m971 scFv comprises a V_{L} and a V_{H}. In certain embodiments, the m971 scFv V_{H} comprises a CDR1 region, a CDR2 region, and a CDR3 region. In this regard, the m971 scFv V_{H} comprises one or more of a V_{H} CDR1 region having the amino acid sequence of SEQ ID NO: 36, a V_{H} CDR2 region comprising the amino acid sequence of SEQ ID NO: 38, and a V_{H} CDR3 region comprising the amino acid sequence of SEQ ID NO: 40. In some embodiments, m971 scFv V_{H} comprises each amino acid sequence of SEQ ID NOs: 36, 38, and 40.

In certain embodiments, the m971 scFv V_{L} comprises a CDR1 region, a CDR2 region, and a CDR3 region. In this regard, the m971 scFv V_{L} comprises one or more of a V_{L} CDR1 region having the amino acid sequence of SEQ ID NO: 43, a V_{L} CDR2 region comprising the amino acid sequence of SEQ ID NO: 45, and a V_{L} CDR3 region comprising the amino acid sequence of SEQ ID NO: 47. In some embodiments, m971 scFv V_{L} comprises each amino acid sequence of SEQ ID NOs: 43, 45, and 47.

In an embodiment, the second antigen binding domain is an anti-CD22 antigen binding domain and comprises each amino acid sequence of SEQ ID NOs: 36, 38, 40, 43, 45, and 47.

The m971 scFv V_{H} may comprise, consist of, or consist essentially of the amino acid sequence of SEQ ID NO: 49. The m971 scFv V_{L} may comprise, consist of, or consist essentially of the amino acid sequence of SEQ ID NO: 50. Accordingly, in an embodiment, second antigen binding domain comprises a V_{H} comprising the amino acid sequence of SEQ ID NO: 49 and/or a V_{L} comprising the amino acid sequence of SEQ ID NO: 50. In an embodiment, the second antigen binding domain comprises amino acid sequences of both SEQ ID NO: 49 and 50.

The amino acid sequences for m97 1 scFv and its components (V_{L} 104, V_{H} 106) are provided in Table 3.

TABLE 3
Amino acid sequences for CD22-specific m971 scFv

| Description | Sequence | SEQ ID NO: |
|---|---|---|
| V_{H}-FR1 | QVQLQQSGPGLVKPSQTLSLTCAIS | 35 |
| V_{H}-CDR1 | GDSVSSNSAA | 36 |
| V_{H}-FR2 | WNWIRQSPSRGLEWLGR | 37 |
| V_{H}-CDR2 | TYYRSKWYN | 38 |
| V_{H}-FR3 | NDYAVSVKSRITINPDTSKNQFSLQLNSVTPEDTAVYYC | 39 |
| V_{H}-CDR3 | AREVTGDLEDAFDI | 40 |
| V_{H}-FR4 | WGQGTMVTVSS | 41 |
| V_{L}-FR1 | DIQMTQSPSSLSASVGDRVTITCRAS | 42 |
| V_{L}-CDR1 | QTIWSY | 43 |
| V_{L}-FR2 | LNWYQQRPGKAPNLLIY | 44 |
| V_{L}-CDR2 | AAS | 45 |
| V_{L}-FR3 | SLQSGVPSRFSGRGSGTDFTLTISSLQAEDFATYYC | 46 |
| V_{L}-CDR3 | QQSYSIPQT | 47 |
| V_{L}-FR4 | FGQGTKLEIK | 48 |
| V_{H} | QVQLQQSGPGLVKPSQTLSLTCAISGDSVSSNSAAWNWIRQ | 49 |
| | SPSRGLEWLGRTYYRSKWYNDYAVSVKSRITINPDTSKNQ | |
| | FSLQLNSVTPEDTAVYYCAREVTGDLEDAFDIWGQGTMVT | |
| | VSS | |
| V_{L} | DIQMTQSPSSLSASVGDRVTITCRASQTIWSYLNWYQQRPG | 50 |
| | KAPNLLIYAASSLQSGVPSRFSGRGSGTDFTLTISSLQAEDF | |
| | ATYYCQQSYSIPQTFGQGTKLEIK | |

In certain embodiments, the scFv of the second antigen binding domain 110 is derived from the CD33 antibody HuM195. The antigen binding domain of HuM195 specifically binds to CD33. In some embodiments, the second antigen binding domain 110 comprises, consists of, or consists essentially of the V_{H} and V_{L} of HuM195 scFv. In one embodiment, the second antigen binding domain 110 is formed by the V_{H} and V_{L} of HuM195 scFv.

The HuM195 scFv comprises a V_{L} and a V_{H}. In certain embodiments, the HuM195 scFv V_{H} comprises a CDR1 region, a CDR2 region, and a CDR3 region.

In certain embodiments, the HuM195 scFv V_{L} comprises a CDR1 region, a CDR2 region, and a CDR3 region.

The HuM195 scFv V_{H} may comprise, consist of, or consist essentially of the amino acid sequence of SEQ ID NO: 51. The HuM195 scFv V_{L} may comprise, consist of, or consist essentially of the amino acid sequence of SEQ ID NO: 52. Accordingly, in an embodiment, second antigen binding domain comprises a V_{H} comprising the amino acid sequence of SEQ ID NO: 51 and/or a V_{L} comprising the amino acid sequence of SEQ ID NO: 52. In an embodiment, the second antigen binding domain comprises amino acid sequences of both SEQ ID NO: 51 and 52.

The amino acid sequences for HuM195 components V_{L} 104, V_{H} 106 are provided in Table 4.

TABLE 4
Amino acid sequences for CD33-specific HuM195 scFv

| Description | Sequence | SEQ ID NO: |
|---|---|---|
| V_{H} | MEWSWVFLFFLSVTTGVHSEVQLVQSGAEVKKPGSSVKVS | 51 |
| | CKASGYTITDSNIHWVRQAPGQSLEWIGYIYPYNGGTDYN | |
| | QKFKNRATLTVDNPTNTAYMELSSLRSEDTDFYYCVNGNP | |
| | WLAYWGQGTLVTVSSASTKGP | |
| V_{L} | MSVPTQVLGLLLLWLTDARCDIQLTQSPSTLSASVGDRVTI | 52 |
| | TCRASESLDNYGIRFLTWFQQKPGKAPKLLMYAASNQGSG | |
| | VPSRFSGSGSGTEFTLTISSLQPDDFATYYCQQTKEVPWSFG | |
| | QGTKVEVKRT | |

In certain embodiments, the scFv of the second antigen binding domain 110 is derived from the CD33 antibody M195. The antigen binding domain of HuM195 specifically binds to CD33. In some embodiments, the second antigen binding domain 110 comprises, consists of, or consists essentially of the V_{H} and V_{L} of M195 scFv. In one embodiment, the second antigen binding domain 110 is formed by the V_{H} and V_{L} of M195 scFv.

The M195 scFv comprises a V_{L} and a V_{H}. In certain embodiments, the HuM195 scFv V_{H} comprises a CDR1 region, a CDR2 region, and a CDR3 region.

In certain embodiments, the M195 scFv V_{L} comprises a CDR1 region, a CDR2 region, and a CDR3 region.

The M195 scFv V_{H} may comprise, consist of, or consist essentially of the amino acid sequence of SEQ ID NO: 53. The M195 scFv V_{L} may comprise, consist of, or consist essentially of the amino acid sequence of SEQ ID NO: 54. Accordingly, in an embodiment, second antigen binding domain comprises a V_{H} comprising the amino acid sequence of SEQ ID NO: 53 and/or a V_{L} comprising the amino acid sequence of SEQ ID NO: 54. In an embodiment, the second antigen binding domain comprises amino acid sequences of both SEQ ID NO: 53 and 54.

In certain embodiments, the scFv of the second antigen binding domain 110 is derived from the CD33 antibody h-p67.6. The antigen binding domain of h-p67.6 specifically binds to CD33. In some embodiments, the second antigen binding domain 110 comprises, consists of, or consists essentially of the V_{H} and V_{L} of h-p67.6 scFv. In one embodiment, the second antigen binding domain 110 is formed by the V_{H} and V_{L} of h-p67.6 scFv.

The h-p67.6 scFv V_{H} may comprise, consist of, or consist essentially of the amino acid sequence of SEQ ID NO: 55. In particular embodiments, the h-p67.6 scFv V_{H} is SEQ ID NO: 55. The h-p67.6 scFv V_{L} may comprise, consist of, or consist essentially of the amino acid sequence of SEQ ID NO: 56. In particular embodiments, the h-p67.6 scFv V_{L} is SEQ ID NO: 56. Accordingly, in an embodiment, the second antigen binding domain comprises a V_{H} comprising the amino acid sequence of SEQ ID NO: 55 and/or a V_{L} comprising the amino acid sequence of SEQ ID NO: 56. In an embodiment, the second antigen binding domain comprises amino acid sequences of both SEQ ID NO: 55 and 56.

In certain embodiments, the scFv of the second antigen binding domain 110 is derived from the CD123 antibody 26292. The antigen binding domain of 26292 specifically binds to CD123. In some embodiments, the second antigen binding domain 110 comprises, consists of, or consists essentially of the V_{H} and V_{L} of 26292 scFv. In one embodiment, the second antigen binding domain 110 is formed by the V_{H} and V_{L} of 26292 scFv.

The 26292 scFv comprises a V_{L} and a V_{H}. In certain embodiments, the 26292 scFv V_{H} comprises a CDR1 region, a CDR2 region, and a CDR3 region. In this regard, the 26292 scFv V_{H} comprises one or more of a V_{H} CDR1 region having the amino acid sequence of SEQ ID NO: 89, a V_{H} CDR2 region comprising the amino acid sequence of SEQ ID NO: 90, and a V_{H} CDR3 region comprising the amino acid sequence of SEQ ID NO: 91. In some embodiments, 26292 scFv V_{H} comprises each amino acid sequence of SEQ ID NOs: 89, 90, and 91.

In certain embodiments, the 26292 scFv V_{L} comprises a CDR1 region, a CDR2 region, and a CDR3 region. In this regard, the 26292 scFv V_{L} comprises one or more of a V_{L} CDR1 region having the amino acid sequence of SEQ ID NO: 93, a V_{L} CDR2 region comprising the amino acid sequence of SEQ ID NO: 94, and a V_{L} CDR3 region comprising the amino acid sequence of SEQ ID NO: 95. In some embodiments, 26292 scFv V_{L} comprises each amino acid sequence of SEQ ID NOs: 93, 94, and 95.

In an embodiment, the second antigen binding domain is an anti-CD123 antigen binding domain and comprises each amino acid sequence of SEQ ID NOs: 89, 90, 91, 93, 94 and 95.

The 26292 scFv V_{H} may comprise, consist of, or consist essentially of the amino acid sequence of SEQ ID NO: 88. The 26292 scFv V_{L} may comprise, consist of, or consist essentially of the amino acid sequence of SEQ ID NO: 92. Accordingly, in an embodiment, the second antigen binding domain comprises a V_{H} comprising the amino acid sequence of SEQ ID NO: 88 and/or a V_{L} comprising the amino acid sequence of SEQ ID NO: 92. In an embodiment, the second antigen binding domain comprises amino acid sequences of both SEQ ID NO: 88 and 92.

The amino acid sequences for 26292 scFv and its components (V_{L} 114, V_{H} 112) are provided in Table 5.

TABLE 5
Amino acid sequences for CD123-specific 26292 scFv

| | | SEQ ID |
|---|---|---|
| Description | Sequences | NO: |
| V_{H} | QVQLQQPGAELVRPGASVKLSCKASGYTFTSYWMNWVK | 88 |
| | QRPDQGLEWIGRIDPYDSETHYNQKFKDKAILTVDKSSST | |
| | AYMQLSSLTSEDSAVYYCARGNWDDYWGQGTTLTVSS | |
| V_{H}-CDR1 | SYWMN | 89 |
| V_{H}-CDR2 | RIDPYDSETHYNQKFKD | 90 |
| V_{H}-CDR3 | GNWDDY | 91 |
| V_{L} | DVQITQSPSYLAASPGETITINCRASKSISKDLAWYQEKPG | 92 |
| | KTNKLLIYSGSTLQSGIPSRFSGSGSGTDFTLTISSLEPEDFA | |
| | MYYCQQHNKYPYTFGGGTKLEIK | |
| V_{L}-CDR1 | RASKSISKDLA | 93 |
| V_{L}-CDR2 | SGSTLQS | 94 |
| V_{L}-CDR3 | QQHNKYPYT | 95 |

In certain embodiments, the scFv of the second antigen binding domain 110 is derived from the CD123 antibody 32701. The antigen binding domain of 32701 specifically binds to CD123. In some embodiments, the second antigen binding domain 110 comprises, consists of, or consists essentially of the V_{H} and V_{L} of 32701 scFv. In one embodiment, the second antigen binding domain 110 is formed by the V_{H} and V_{L} of 32701 scFv.

The 32701 scFv comprises a V_{L} and a V_{H}. In certain embodiments, the 32701 scFv V_{H} comprises a CDR1 region, a CDR2 region, and a CDR3 region. In this regard, the 32701 scFv V_{H} comprises one or more of a V_{H} CDR1 region having the amino acid sequence of SEQ ID NO: 97, a V_{H} CDR2 region comprising the amino acid sequence of SEQ ID NO: 98, and a V_{H} CDR3 region comprising the amino acid sequence of SEQ ID NO: 99. In some embodiments, 32701 scFv V_{H} comprises each amino acid sequence of SEQ ID NOs: 97, 98, and 99.

In certain embodiments, the 32701 scFv V_{L} comprises a CDR1 region, a CDR2 region, and a CDR3 region. In this regard, the 32701 scFv V_{L} comprises one or more of a V_{L} CDR1 region having the amino acid sequence of SEQ ID NO: 101, a V_{L} CDR2 region comprising the amino acid sequence of SEQ ID NO: 102, and a V_{L} CDR3 region comprising the amino acid sequence of SEQ ID NO: 103. In some embodiments, 32701 scFv V_{L} comprises each amino acid sequence of SEQ ID NOs: 101, 102, and 103.

In an embodiment, the second antigen binding domain is an anti-CD123 antigen binding domain and comprises each amino acid sequence of SEQ ID NOs: 97, 98, 99, 101, 102 and 103.

The 32701 scFv V_{H} may comprise, consist of, or consist essentially of the amino acid sequence of SEQ ID NO: 96. The 32701 scFv V_{L} may comprise, consist of, or consist essentially of the amino acid sequence of SEQ ID NO: 100. Accordingly, in an embodiment, the second antigen binding domain comprises a V_{H} comprising the amino acid sequence of SEQ ID NO: 96 and/or a V_{L} comprising the amino acid sequence of SEQ ID NO: 100. In an embodiment, the second antigen binding domain comprises amino acid sequences of both SEQ ID NO: 96 and 100.

The amino acid sequences for 32701 scFv and its components (V_{L} 114, V_{H} 112) are provided in Table 6.

TABLE 6
Amino acid sequences for CD123-specific 32701 scFv

| | | SEQ ID |
|---|---|---|
| Description | Sequences | NO: |
| V_{H} | QIQLVQSGPELKKPGETVKISCKTSGYVFTNYGMNWVKQAPG | 96 |
| | KGFKWMGWMNTNTGEPTSLEDFKGRFAFSLETSASTAYLQIN | |
| | NLKNDDTATYFCARSGGYDPMDYWGQGTSVTVSS | |
| V_{H}-CDR1 | NYGMN | 97 |
| V_{H}-CDR2 | WMNTNTGEPTSLEDFKG | 98 |
| V_{H}-CDR3 | SGGYDPMDY | 99 |
| V_{L} | DIVLTQSPASLAVSPGQRATISCRASESVDNYGNTFMHWYQQK | 100 |
| | PGQPPKLLIYRASNLESGIPARFSGSDSRTDFTLTINPVEADDVA | |
| | TYYCQQSKEDPPTFGGTKLELK | |
| V_{L}-CDR1 | RASESVDNYGNTFMH | 101 |
| V_{L}-CDR2 | RASNLES | 102 |
| V_{L}-CDR3 | QQSKEDPPT | 103 |

In certain embodiments, the scFv of the second antigen binding domain 110 is derived from the CD123 antibody 32716. The antigen binding domain of 32716 specifically binds to CD123. In some embodiments, the second antigen binding domain 110 comprises, consists of, or consists essentially of the V_{H} and V_{L} of 32716 scFv. In one embodiment, the second antigen binding domain 110 is formed by the V_{H} and V_{L} of 32716 scFv.

The 32716 scFv comprises a V_{L} and a V_{H}. In certain embodiments, the 32716 scFv V_{H} comprises a CDR1 region, a CDR2 region, and a CDR3 region. In this regard, the 32716 scFv V_{H} comprises one or more of a V_{H} CDR1 region having the amino acid sequence of SEQ ID NO: 105, a V_{H} CDR2 region comprising the amino acid sequence of SEQ ID NO: 106, and a V_{H} CDR3 region comprising the amino acid sequence of SEQ ID NO: 107. In some embodiments, 32716 scFv V_{H} comprises each amino acid sequence of SEQ ID NOs: 105, 106, and 107.

In certain embodiments, the 32716 scFv V_{L} comprises a CDR1 region, a CDR2 region, and a CDR3 region. In this regard, the 32716 scFv V_{L} comprises one or more of a V_{L} CDR1 region having the amino acid sequence of SEQ ID NO: 109, a V_{L} CDR2 region comprising the amino acid sequence of SEQ ID NO: 110, and a V_{L} CDR3 region comprising the amino acid sequence of SEQ ID NO: 111. In some embodiments, 32716 scFv V_{L} comprises each amino acid sequence of SEQ ID NOs: 109, 110, and 111.

In an embodiment, the second antigen binding domain is an anti-CD123 antigen binding domain and comprises each amino acid sequence of SEQ ID NOs: 105, 106, 107, 109, 110 and 111.

The 32716 scFv V_{H} may comprise, consist of, or consist essentially of the amino acid sequence of SEQ ID NO: 104. The 32716 scFv V_{L} may comprise, consist of, or consist essentially of the amino acid sequence of SEQ ID NO: 108. Accordingly, in an embodiment, the second antigen binding domain comprises a V_{H} comprising the amino acid sequence of SEQ ID NO: 104 and/or a V_{L} comprising the amino acid sequence of SEQ ID NO: 108. In an embodiment, the second antigen binding domain comprises amino acid sequences of both SEQ ID NO: 104 and 108.

The amino acid sequences for 32716 scFv and its components (V_{L} 114, V_{H} 112) are provided in Table 7.

TABLE 7
Amino acid sequences for CD123-specific 32716 scFv

| Description | Sequences | SEQ ID NO: |
|---|---|---|
| V_{H} | QIQLVQSGPELKKPGETVKISCKASGYIFTNYGMNWVKQAP | 104 |
| | GKSFKWMGWINTYTGESTYSADFKGRFAFSLETSASTAYLHI | |
| | NDLKNEDTATYFCARSGGYDPMDYWGQGTSVTVSS | |
| V_{H}-CDR1 | NYGMN | 105 |
| V_{H}-CDR2 | WINTYTGESTYSADFKG | 106 |
| V_{H}-CDR3 | SGGYDPMDY | 107 |
| V_{L} | DIVLTQSPASLAVSLGQRATISCRASESVDNYGNTFMHWYQ | 108 |
| | QKPGQPPKLLIYRASNLESGIPARFSGSGSRTDFTLTINPVEAD | |
| | DVATYYCQQSNEDPPTFGAGTKELK | |
| V_{L}-CDR1 | RASESVDNYGNTFMH | 109 |
| V_{L}-CDR2 | RASNLES | 110 |
| V_{L}-CDR3 | QQSNEDPPT | 111 |

In certain embodiments, the scFv of the second antigen binding domain 110 is derived from the CD123 antibody 32703. The antigen binding domain of 32703 specifically binds to CD123. In some embodiments, the second antigen binding domain 110 comprises, consists of, or consists essentially of the V_{H} and V_{L} of 32703 scFv. In one embodiment, the second antigen binding domain 110 is formed by the V_{H} and V_{L} of 32703 scFv.

The 32703 scFv comprises a V_{L} and a V_{H}. In certain embodiments, the 32703 scFv V_{H} comprises a CDR1 region, a CDR2 region, and a CDR3 region. In this regard, the 32703 scFv V_{H} comprises one or more of a V_{H} CDR1 region having the amino acid sequence of SEQ ID NO: 113, a V_{H} CDR2 region comprising the amino acid sequence of SEQ ID NO: 114, and a V_{H} CDR3 region comprising the amino acid sequence of SEQ ID NO: 115. In some embodiments, 32703 scFv V_{H} comprises each amino acid sequence of SEQ ID NOs: 113, 114, and 115.

In certain embodiments, the 32703 scFv V_{L} comprises a CDR1 region, a CDR2 region, and a CDR3 region. In this regard, the 32703 scFv V_{L} comprises one or more of a V_{L} CDR1 region having the amino acid sequence of SEQ ID NO: 117, a V_{L} CDR2 region comprising the amino acid sequence of SEQ ID NO: 118, and a V_{L} CDR3 region comprising the amino acid sequence of SEQ ID NO: 119. In some embodiments, 32703 scFv V_{L} comprises each amino acid sequence of SEQ ID NOs: 117, 118 and 119.

In an embodiment, the second antigen binding domain is an anti-CD123 antigen binding domain and comprises each amino acid sequence of SEQ ID NOs: 113, 114, 115, 117, 118 and 119.

The 32703 scFv V_{H} may comprise, consist of, or consist essentially of the amino acid sequence of SEQ ID NO: 112. The 32703 scFv V_{L} may comprise, consist of, or consist essentially of the amino acid sequence of SEQ ID NO: 116. Accordingly, in an embodiment, the second antigen binding domain comprises a V_{H} comprising the amino acid sequence of SEQ ID NO: 112 and/or a V_{L} comprising the amino acid sequence of SEQ ID NO: 116. In an embodiment, the second antigen binding domain comprises amino acid sequences of both SEQ ID NO: 112 and 116.

The amino acid sequences for 32703 scFv and its components (V_{L} 114, V_{H} 112) are provided in Table 8.

TABLE 8
Amino acid sequences for CD123-specific 32703 scFv

| Description | Sequences | SEQ ID NO: |
|---|---|---|
| V_{H} | QVQLQQPGAELVKPGAPVKLSCKASGYTFTNYWMNWIKQ | 112 |
| | RPGRGLEWIGRIDPSDSESHYNQKFKDKATLTVDKSSNTAY | |
| | IQLSSLTSEDSAVYYCARYDYDDTMDYWGQGTSVTVSS | |
| V_{H}-CDR1 | NYWMN | 113 |
| V_{H}-CDR2 | RIDPSDSESHYNQKFKD | 114 |
| V_{H}-CDR3 | YDYDDTMDY | 115 |
| V_{L} | DIVMTQAAPSVPVTPGESVSISCRSNKSLLHSNGNTYLYWF | 116 |
| | LQRPGQSPQLLIYRMSNLASGVPDRFSGSGSGTAFTLRISRV | |
| | EAEDVGVYYCMQHLEYPYTFGGGTKLEIK | |
| V_{L}-CDR1 | RSNKSLLHSNGNTYLY | 117 |
| V_{L}-CDR2 | RMSNLAS | 118 |
| V_{L}-CDR3 | MQHLEYPYT | 119 |

The second antigen binding domain 110 depicted in FIG. 2A comprises a V_{L} 114, and a V_{H} 112, but is missing an intra-binding domain linker. Whether the second antigen binding domain 110 comprises intra-binding domain linker 116 is dependent on the configuration of the bivalent CAR as provided by the present disclosure. As illustrated in FIG. 2A, V_{L} 114 and V_{H} 116 are each linked to one of V_{L} 104 or V_{H} 106 of the first FLT3-specific antigen binding domain 102 via an inter-binding domain linker.

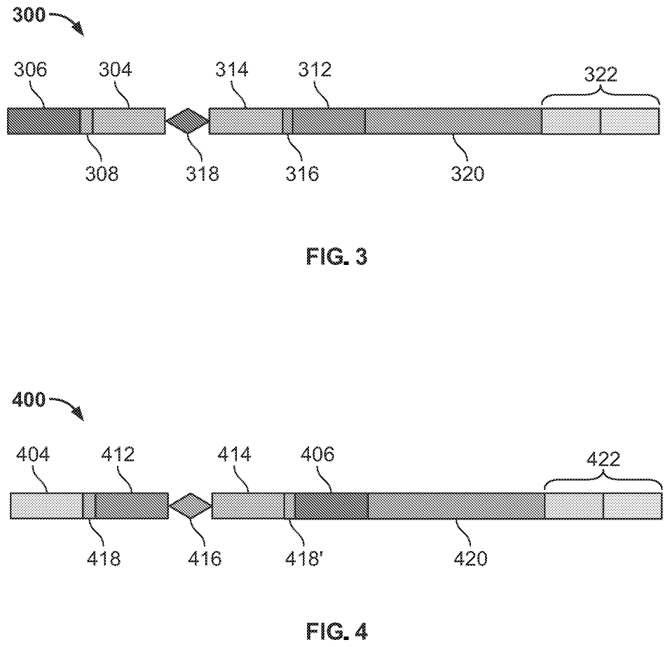

Also provided by the present disclosure are expression constructs encoding the bivalent CARs disclosed and described herein. FIG. 3 is a schematic depicting an expression construct 300 encoding a tandem bivalent CAR according to one embodiment, wherein polynucleotides encoding each element of the bivalent CAR are arranged in sequential order. For example, and referring to the polynucleotides of FIG. 3 and polypeptides of FIG. 1, polynucleotides encoding the anti-FLT3 antigen binding domain 102, the inter-binding domain linker 118, and the second antigen binding domain 110 may be ordered as follows: i) a polynucleotide 306 encoding an anti-FLT3 antigen binding domain V_{H} 106, ii) a polynucleotide 308 encoding an anti-FLT3 antigen binding domain intra-binding domain 108, iii) a polynucleotide 304 encoding an anti-FLT3 antigen binding domain V_{L} 104, iv) polynucleotide 318 encoding inter-binding domain linker 118, v) a polynucleotide 314 encoding second antigen binding domain V_{L} 114, vi) a polynucleotide 316 encoding a second antigen binding domain intra-binding domain 116, vii) a polynucleotide 312 encoding anti-FLT3 antigen binding domain V_{H} 112, viii) a polynucleotide 320 encoding transmembrane domain 120, and ix) a polynucleotide 322 encoding intracellular T-cell signaling domain 122. An expression construct having this sequential ordering of polynucleotides will result in the expression of the tandem bivalent CAR depicted by FIG. TA, in which the first anti-FLT3 antigen binding domain is distal to the transmembrane domain. Alternatively, the polynucleotides can be ordered so that the anti-FLT3 antigen binding domain is proximal to the transmembrane domain, as depicted in FIG. 1B. In either configuration, the order of the polynucleotides encoding the V_{H} and V_{L} for one or both antigen binding domains can be reversed.

FIG. 4 is a schematic depicting an expression construct 400 encoding a loop bivalent CAR according to one embodiment, wherein polynucleotides encoding one of the binding domains are separated by polynucleotides encoding the other binding domain. For example, and referring to the polynucleotides of FIG. 4 and polypeptides of FIG. 2, a polynucleotides encoding the anti-FLT3 antigen binding domain 102, the inter-binding domain linker 118, and the second antigen binding domain 110 may be orders as follows: i) a polynucleotide 404 encoding anti-FLT3 antigen binding domain V_{L} 104, ii) a polynucleotide 418 encoding inter-binding domain linker 118, iii) a polynucleotide 412 encoding second antigen binding domain V_{H} 112, iv) a polypeptide 416 encoding second antigen binding domain intra-binding domain 116, v) a polynucleotide 414 encoding second antigen binding domain V_{L} 114, vi) a polynucleotide 418′ encoding inter-binding domain linker 118′, vii) a poly nucleotide 406 encoding anti-FLT3 antigen binding domain V_{H} 106, viii) a poly nucleotide 420 encoding transmembrane domain 120, and ix) a polynucleotide 422 intracellular T-cell signaling domain 122. An expression construct having such an ordering of polynucleotides will result in the expression of the loop bivalent CAR depicted by FIG. 2A, in which the first anti-FLT3 antigen binding domain is distal to the transmembrane domain. Alternatively, the polynucleotides can be ordered so that the anti-FLT3 antigen binding domain is proximal to the transmembrane domain, as depicted in FIG. 2A. In either configuration, the order of the polynucleotides encoding the V_{H} and V_{L} for one or both antigen binding domains can be reversed.

In some embodiments, the first anti-FLT3 antigen binding domain and/or the second antigen binding domain comprise an intra-binding domain linker, linking the V_{H} and the V_{L} of the binding domain. In some embodiments, the linker is the original linker from the antibody-derived scFv. In one embodiment, for example, the intra-binding domain linker of the first anti-FLT3 is the linker from NC7 scFv and has the amino acid sequence of SEQ ID NO: 8. In some embodiments, the intra-binding domain linker may comprise any suitable linker amino acid sequence. In some embodiments, the intra-binding domain linker is a Gly/Ser linker of about 1 to about 100, from about 3 to about 50, from about 5 to about 30, from about 5 to about 20, or from about 3 to about 8 amino acids in length. Accordingly, the Gly/Ser linker consists of glycine and/or serine residues. In some embodiments, the intra-binding domain Gly/Ser linker is a peptide having the formula (Xaal)n wherein each amino acid residue Xaal is selected independently from glycine and serine, and n is an integer from 3 to 8. In some embodiments, the Gly/Ser intra-binding domain linker comprises the amino acid sequence of SEQ ID NO: 67 (GSTSGSGKPGSGEGSTKG) or 68 (GGGGS). In some embodiments, the intra-binding domain linker is a peptide having the amino acid formula [GGGGS (SEQ ID NO: 68)]ₘ, wherein m is an integer from 1 to 10, from 2 to 8, or from 3 to 5. In some embodiments, m is 5. In some embodiments, the inter-binding domain linker comprises the amino acid sequence of SEQ ID NO: 69 (GGGGSGGGGSGGGGSGGGGSGGGGS). In some aspects, an intra-binding domain linker can comprise a sequence of any of SEQ ID NOs: 61-66.

In some embodiments, the first anti-FLT3 antigen binding domain is joined to the second antigen binding domain via one or more inter-binding domain linkers. In the tandem bivalent CAR configuration, a single inter-binding domain linker joins either the V_{H} or V_{L} of the first anti-FLT3 antigen binding domain to either the V_{H} or V_{L} of the second antigen binding domain. In the loop bivalent CAR configuration, a first inter-binding domain linker joins the V_{H} of the first anti-FLT3 antigen binding domain to either the V_{H} or V_{L} of the second antigen binding domain and a second inter-binding domain linker joins the V_{L} of the first anti-FLT3 antigen binding domain to the V_{H} or V_{L} that is not joined to the V_{H} of the first anti-FLT3 antigen binding domain. In some embodiments, the inter-binding domain linker comprises a Gly/Ser linker. In some embodiments, the inter-binding domain linker is a peptide having the amino acid formula [GGGGS (SEQ ID NO: 68)]ₘ, wherein m is an integer from 1 to 10, from 2 to 8, or from 3 to 5. In some embodiments, m is 5. In some embodiments, the inter-binding domain linker comprises the amino acid sequence of SEQ ID NO: 69 (GGGGSGGGGSGGGGSGGGGSGGGGS). In some aspects, an intra-binding domain linker can comprise a sequence of any of SEQ ID NOs: 61-66.

In some embodiments, the intra-binding domain linker of one or both binding domains and/or the inter-binding domain linker(s) can be optimized to maximize bivalent surface CAR protein expression in a host cell and/or maximize antigen binding by the two antigen binding domains.

In certain embodiments, one of the antigen binding domains of the bivalent CAR comprises a leader sequence or signal peptide. In some embodiments, the leader sequence or signal peptide is positioned at the amino terminus of either the first anti-FLT3 antigen binding domain or of the second antigen binding domain. The leader sequence can be any suitable leader sequence such as, for example, a leader sequence from human GM-CSF comprising, consisting of, or consisting essentially of the amino acid sequence of SEQ ID NO: 70 (LLLVTSLLLCELPHPAFLLIP). In an embodiment, the leader sequence from human GM-CSF is the amino acid sequence of SEQ ID NO: 70. In some embodiments, the leader sequence further comprises a start methionine. Although not required, in certain embodiments the leader sequence or signal peptide facilitates expression of the bivalent CAR on the surface of a host cell. In some embodiments, the leader sequence or signal peptide is cleaved off of the CAR upon expression of the CAR at the cell surface. Accordingly, in some embodiments, the bivalent CAR lacks a leader sequence.

In some embodiments, the bivalent CAR comprises a transmembrane (TM) domain. In some embodiments, the TM domain is a CD8a TM domain. In particular embodiments, the TM domain is a human CD8a TM domain. In some embodiments, the CD8 TM domain comprises, consists of, or consists essentially of a peptide having the amino acid sequence of SEQ ID NO: 71 (IYIWAPLAGTCGVLLLSLVITLYC). In an embodiment, the CD8a TM domain is a peptide having the amino acid sequence of SEQ ID NO: 71. In some embodiments, the TM domain is a CD28 TM domain. In particular embodiments, the TM domain is a human CD28 TM domain. In some embodiments, the CD28 TM domain comprises, consists of, or consists essentially of a peptide having the amino acid sequence of SEQ ID NO: 79 (FWVLVVVGGVLACYSLLVTVAFIIFWV). In an embodiment, the CD8a TM domain is a peptide having the amino acid sequence of SEQ ID NO: 79.

In some embodiments, the bivalent CAR comprises a hinge domain. In some embodiments, the hinge domain is a CD8a hinge domain. In particular embodiments, the hinge domain is a human CD8a hinge domain. In some embodiments, the CD8a hinge domain comprises, consists of, or consists essentially of a peptide having the amino acid sequence of SEQ ID NO: 72 (TTTPAPRPPTPAPTIASQPLSLRPEACRPAAGGAVHTRGLDFACD). In an embodiment, the CD8a hinge domain is a peptide having the amino acid sequence of SEQ ID NO: 72. In some embodiments, the hinge domain is a CD28 hinge domain. In particular embodiments, the hinge domain is a human CD28 hinge domain. In some embodiments, the CD28 hinge domain comprises, consists of, or consists essentially of a peptide having the amino acid sequence of SEQ ID NO: 80 (TSAAAIEVMYPPPYLDNEKSNGTIIHVKGKHLCPSPLFPGPSKP). In an embodiment, the CD28 TM domain is a peptide having the amino acid sequence of SEQ ID NO: 80.

In some embodiments, the hinge domain joins the two antigen binding domains to the transmembrane domain. In those embodiments that do not include a hinge domain, the two antigen binding domains are joined to the transmembrane domain via a suitable linker.

In some embodiments, the bivalent CAR comprises one or more spacer peptides. The spacer may between any aforementioned domain or region of the CAR. In an embodiment, the CAR comprises an IgG heavy chain constant domain (CH2CH3) spacer. In another embodiment, the spacer can be between the scFv and the transmembrane domain.

In some embodiments, the bivalent CAR comprises an intracellular T-cell signaling domain. In some embodiments, the intracellular T-cell signaling domain comprises a 4-1BB intracellular T-cell signaling sequence. 4-1BB, also known as CD137, transmits a potent costimulatory signal to T-cells, promoting differentiation and enhancing long-term survival of T lymphocytes. In some embodiments, the 4-1BB intracellular T-cell signaling sequence is human. In some embodiments, the 4-1BB intracellular T-cell signaling sequence comprises, consists of, or consists essentially of the amino acid sequence of SEQ ID NO: 73 (KRGRKKLLYIFKQPFMRPVQTTQEEDGCSCRFPEEEEGGCEL). In an embodiment, the 4-1BB intracellular T-cell signaling sequence is the amino acid sequence of SEQ ID NO: 73. In some embodiments, the intracellular T-cell signaling domain comprises a CD28 intracellular T-cell signaling sequence. In some embodiments, the CD28 intracellular T-cell signaling sequence is human. In some embodiments, the CD28 intracellular T-cell signaling sequence comprises, consists of, or consists essentially of the amino acid sequence of SEQ ID NO: 81 (KRGRKKLLYIFKQPFMRPVQTTQEEDGCSCRFPEEEEGGCEL). In an embodiment, the 4-1BB intracellular T-cell signaling sequence is the amino acid sequence of SEQ ID NO: 81.

In certain embodiments, the intracellular T-cell signaling domain comprises a CD3ζ intracellular T-cell signaling sequence. CD3ζ associates with T-cell receptors (TCRs) to produce a cellular signal and includes immunoreceptor tyrosine-based activation motifs (ITAMs). In certain embodiments, the CD3ζ intracellular T-cell signaling sequence is human. In particular embodiments, the CD3ζ intracellular T-cell signaling sequence comprises, consists of, or consists essentially of the amino acid sequence of SEQ ID NO: 74 where Xaa at position 14 is glutamine or lysine. In an embodiment, the CD3ζ intracellular T-cell signaling sequence is the amino acid sequence of SEQ ID NO: 74, where Xaa at position 14 is glutamine or lysine.

An intracellular T-cell signaling domain can comprise any number and any combination of costimulatory domains. Costimulatory domains include, but not limited to, 4-1BB costimulatory domains, CD28 costimulatory domains, ICOS stimulatory domains, OX-40 costimulatory domains, CD40L costimulatory domains, CD27 costimulatory domains and TLR2 costimulatory domains. Sequences of the preceding costimulatory domains are known in the art. When more than one costimulatory domain is present in a single T-cell signaling domain, the costimulatory domains can be present in any order.

In some aspects, an intracellular T-cell signaling domain can comprise a CD28 costimulatory domain. In some aspects, an intracellular T-cell signaling domain can comprise a 4-1BB costimulatory domain. In some aspects, an intracellular T-cell signaling domain can comprise a CD28 costimulatory domain and a 4-1BB costimulatory domain. In some aspects, an intracellular T-cell signaling domain can comprise a CD28 costimulatory domain and an OX-40 costimulatory domain.

In some aspects, an intracellular T-cell signaling domain can comprise a CD3ζ intracellular T-cell signaling sequence. In some aspects, an intracellular T-cell signaling domain can comprise a CD3ζ intracellular T-cell signaling sequence and a CD28 costimulatory domain. In some aspects, an intracellular T-cell signaling domain can comprise a CD3ζ intracellular T-cell signaling sequence and a 4-1BB costimulatory domain. In some aspects, an intracellular T-cell signaling domain can comprise a CD3ζ intracellular T-cell signaling sequence, a CD28 costimulatory domain and a 4-1BB costimulatory domain. In some aspects, an intracellular T-cell signaling domain can comprise a CD3ζ intracellular T-cell signaling sequence, a CD28 costimulatory domain and an OX-40 costimulatory domain.

The V_{H} and V_{L} of the anti-FLT3 antigen binding domain and the V_{H} and V_{L} of the second antigen binding domain (i.e., an anti-CD19 binding domain, an anti-CD22 binding domain, an anti-CD33 binding domain, or an anti-CD123 binding domain) can be positioned in any suitable orientation with any of the linkers described herein positioned between the heavy and light chains.

Several iterations of each configuration (e.g., tandem, with first FLT3 antigen binding domain distal to TM domain; loop, with first FLT3 antigen binding domain proximal to TM domain) can be designed, generated, and tested. Inter- and intra-binding domain linker lengths, inter- and intra-binding domain linker sequences, binding domain configuration (i.e., relative to TM domain), V_{H} and V_{L} order and orientation. Each of these factors have an effect on CAR surface expression, antigen binding, or both, demonstrating the challenges of generating bivalent CAR constructs that maintain bispecific activity.

Bicistronic Chimeric Antigen Receptors

Certain embodiments described herein provide a CAR expression construct comprising two complete CAR constructs connected via a cleavable linker. Such CAR constructs are referred to herein as a “bicistronic CAR construct” or a “bicistronic CAR expression construct”. Bicistronic CAR expression constructs of the present disclosure encode a first CAR comprising an anti-FLT3 antigen binding domain and a second CAR comprising an antigen binding domain that is specific for CD19, CD22, CD33 or CD123. Each of the first and second CARs encoded by the bicistronic CAR expression construct also comprise a transmembrane domain and an intracellular signaling domain. The bicistronic CAR expression construct encodes a cleavable linker that links the two CARs until it is cleaved.

Accordingly, in one embodiment the bicistronic CAR construct encodes a first FLT3 CAR and a second CD19 CAR. In another embodiment, the bicistronic CAR construct encodes a first FLT3 CAR and a second CD22 CAR. In yet another embodiment, the bicistronic CAR construct encodes a first FLT3 CAR and a second CD33 CAR. In yet another embodiment, the bicistronic CAR construct encodes a first FLT3 CAR and a second CD123 CAR.

As with the bivalent CARs described herein, the pair of CARs expressed from the bicistronic CAR expression constructs may advantageously provide greater potency and may reduce or prevent cell escape due to loss or reduction in the expression of one of FLT3, CD16, CD22, CD33 or CD123. Bicistronic CAR expression constructs encoding a first CAR comprising a FLT3-specific antigen binding domain and a second CAR comprising a CD19-specific antigen binding domain, or a first CAR comprising a FLT3-specific antigen binding domain and a second CAR comprising a CD22-specific antigen binding domain can be used to treat ALL as well as treat relapsed or refractory ALL in which CD19 or CD22 surface protein expression is lost or significantly reduced. Similarly, bicistronic CAR expression constructs encoding a first CAR comprising a FLT3-specific antigen binding domain and a second CAR comprising a CD33-specific antigen binding domain, or a first CAR comprising a FLT3-specific antigen binding domain and a second CAR comprising a CD123-specific antigen binding domains can be used to treat AML as well as treat relapsed or refractory AML in which surface protein expression of one antigen (e.g. CD33 or CD123 or FLT3) is lost or significantly reduced. Bicistronic CAR expression constructs encoding a first CAR comprising a FLT3-specific antigen binding domain and a second CAR comprising a CD123-specific antigen binding domains can also be used to treat precursor B-cell ALL.

A bicistronic CAR expression construct provides significant advantages over two separate CAR constructs. Namely, a bicistronic CAR expression construct allows for efficient expression of the two CARs from a single vector, allowing for a more efficient transfection of host cells, such as T-cells. The result is a more efficient and simultaneous targeting of FLT3 and CD19, FLT3 and CD22, FLT3 and CD33 or FLT3 and CD123 by the same T-cell. As can be appreciated, greater transfection and targeting efficiency can, and often does, lead to improvements in the treatment of a disease such as ALL or AML.

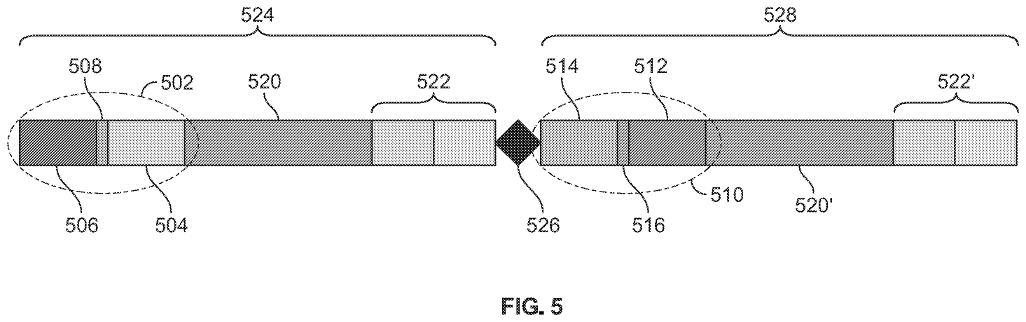

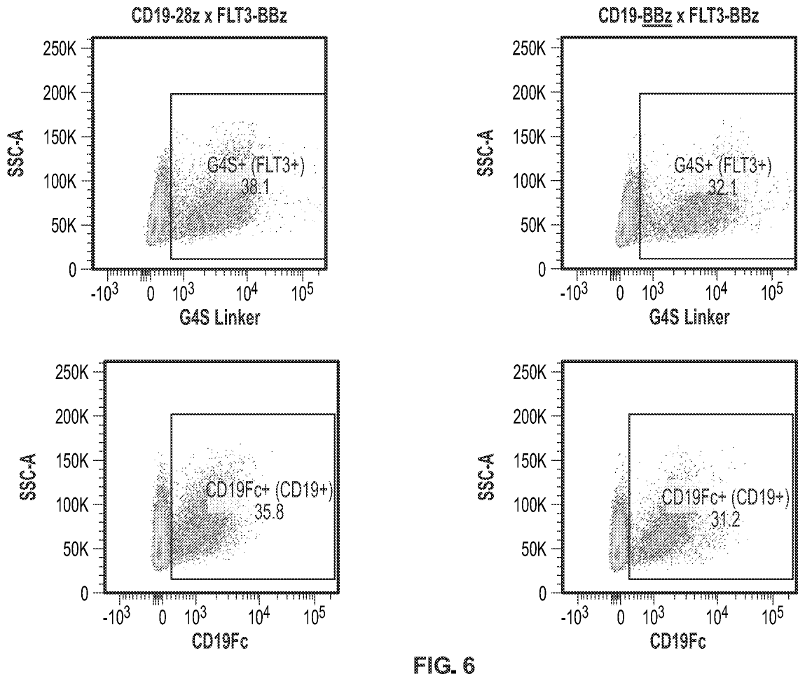

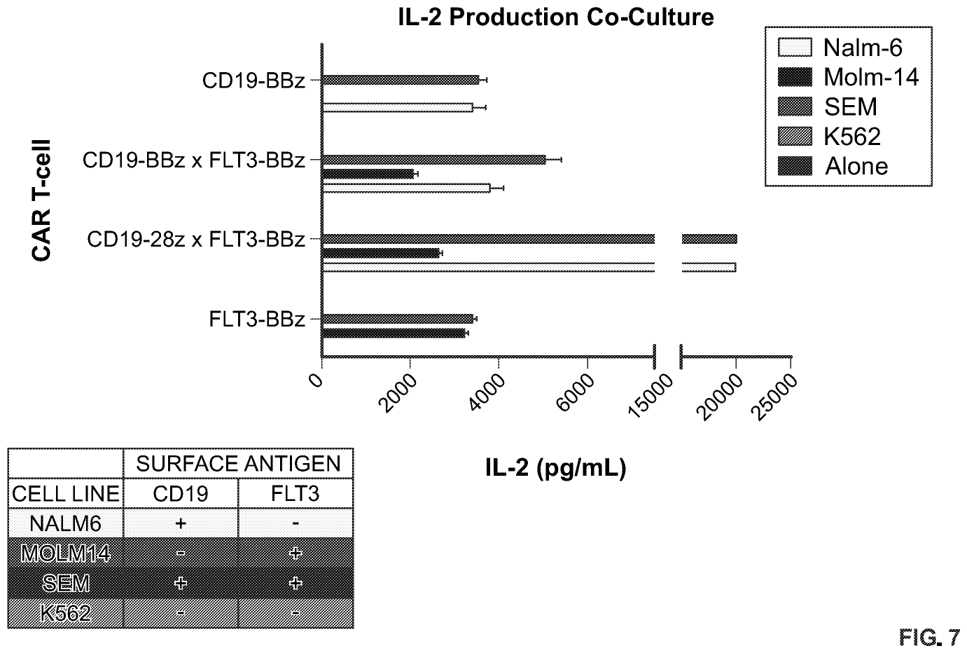

FIG. 5 is a schematic of a representative bicistronic CAR expression construct 500 according to an embodiment of the present disclosure. The depicted bicistronic CAR expression construct 500 comprises: a first group of nucleotide sequences 524, which encodes a first CAR comprising an anti-FLT3 antigen binding domain 502, a transmembrane domain 520, and an intracellular signaling domain 522; a second group of nucleotide sequences 528, which encodes a second CAR comprising a second antigen binding domain 510, a transmembrane domain 520, and an intracellular signaling domain 522; and a cleavable linker nucleotide sequence 526 encoding a cleavable linker, the cleavable linker nucleotide being positioned between the first group of nucleotide sequences and the second group of nucleotide sequences and joins the two group of nucleotide sequences.

The anti-FLT3 antigen binding domain 502 comprises a V_{L} 504, a V_{H} 506, and an intra-binding domain linker 508. The anti-FLT3 antigen binding domain 502 may be derived from a portion of an antibody that specifically recognizes an epitope of FLT3 (e.g., an scFv). The anti-FLT3 antigen binding domain 502 can be derived from any specific FLT3 antibody. In certain embodiments, the anti-FLT3 antigen binding domain 502 is derived from an FLT3 antibody selected from m1006, m1007, and NC7. The antigen binding domain of each of m1006, m1007, and NC7 specifically binds to FLT3. In some embodiments, the anti-FLT3 antigen binding domain 502 comprises, consists of, or consists essentially of the V_{H} and V_{L} of m1006 scFv, the V_{H} and V_{L} of m1007 scFv, or the V_{H} and V_{L} of NC7 scFv. In some embodiments, the anti-FLT3 antigen binding domain 502 is formed by the V_{H} and V_{L} of m1006 scFv, the V_{H} and V_{L} of m1007 scFv, or the V_{H} and V_{L} of V_{H} and V_{L} of NC7 scFv. The V_{H} and V_{L} of each of m1006 scFv, m1007 scFv, and NC7 scFv are fully described elsewhere herein.

The second antigen binding domain 510 of the second CAR, which is encoded by the second group of nucleotide sequences 528, is selected from the group of an anti-CD19 antigen binding domain, an anti-CD22 antigen binding domain, an anti-CD33 antigen binding domain and an anti-CD123 binding domain, and comprises a V_{L} 514, a V_{H} 512, and an intra-binding domain linker 516. The antigen binding domain 510 of the second CAR is derived from a portion of an antibody that specifically recognizes an epitope from one of CD19, CD22, CD33 or CD123 (e.g., an scFv). The antigen binding domain 510 of the second CAR can be derived from any specific antibody that binds CD19, CD22, CD33 or CD123.

In certain embodiments, the scFv of the antigen binding domain 510 of the second CAR is derived from the CD19 antibody FMC63. The antigen binding domain of FMC63 specifically binds to CD19. In some embodiments, the antigen binding domain 510 of the second CAR comprises, consists of, or consists essentially of the V_{H} and V_{L} of FMC63 scFv. In particular embodiments, the antigen domain 510 of the second CAR is formed by the V_{H} and V_{L} of FMC63 scFv. The FMC63 scFv anti-CD19 antigen domain is fully described elsewhere herein.

In certain embodiments, the scFv of the antigen binding domain 510 of the second CAR is derived from the CD22 antibody m971. The antigen binding domain of m971 specifically binds to CD22. In some embodiments, the antigen binding domain 510 of the second CAR comprises, consists of, or consists essentially of the V_{H} and V_{L} of m971 scFv. In particular embodiments, the antigen binding domain 510 of the second CAR is formed by the V_{H} and V_{L} of m971 scFv. The m971 scFv anti-CD22 antigen domain is fully described elsewhere herein.

In certain embodiments, the scFv of the antigen binding domain 510 of the second CAR is derived from the CD123 antibody 26292. The antigen binding domain of 26292 specifically binds to CD123. In some embodiments, the antigen binding domain 510 of the second CAR comprises, consists of, or consists essentially of the V_{H} and V_{L} of 26292 scFv. In particular embodiments, the antigen domain 510 of the second CAR is formed by the V_{H} and V_{L} of 26292 scFv. The 26292 scFv anti-CD123 antigen domain is fully described elsewhere herein.

In certain embodiments, the scFv of the antigen binding domain 510 of the second CAR is derived from the CD123 antibody 32701. The antigen binding domain of 32701 specifically binds to CD123. In some embodiments, the antigen binding domain 510 of the second CAR comprises, consists of, or consists essentially of the V_{H} and V_{L} of 32701 scFv. In particular embodiments, the antigen domain 510 of the second CAR is formed by the V_{H} and V_{L} of 32701 scFv. The 32701 scFv anti-CD123 antigen domain is fully described elsewhere herein.

In certain embodiments, the scFv of the antigen binding domain 510 of the second CAR is derived from the CD123 antibody 32716. The antigen binding domain of 32716 specifically binds to CD123. In some embodiments, the antigen binding domain 510 of the second CAR comprises, consists of, or consists essentially of the V_{H} and V_{L} of 32716 scFv. In particular embodiments, the antigen domain 510 of the second CAR is formed by the V_{H} and V_{L} of 32716 scFv. The 32716 scFv anti-CD123 antigen domain is fully described elsewhere herein.

In certain embodiments, the scFv of the antigen binding domain 510 of the second CAR is derived from the CD123 antibody 32703. The antigen binding domain of 32703 specifically binds to CD123. In some embodiments, the antigen binding domain 510 of the second CAR comprises, consists of, or consists essentially of the V_{H} and V_{L} of 32703 scFv. In particular embodiments, the antigen domain 510 of the second CAR is formed by the V_{H} and V_{L} of 32703 scFv. The 32703 scFv anti-CD123 antigen domain is fully described elsewhere herein.

In certain embodiments, the anti-FLT3 antigen binding domain 502 of the first CAR and the antigen binding domain 510 of the second CAR each comprise an intra-binding domain linker linking the V_{H} and V_{L} of the binding domain. In some embodiments, the linker is the original linker from the antibody-derived scFv. In one embodiment, for example, the intra-binding domain linker of the first anti-FLT3 is the linker from NC7 scFv and has the amino acid sequence of SEQ ID NO: 8. In some embodiments, the intra-binding domain linker may comprise any suitable linker amino acid sequence. In some embodiments, the intra-binding domain linker is a Gly/Ser linker of about 1 to about 100, from about 3 to about 50, from about 5 to about 30, from about 5 to about 20, or from about 3 to about 8 amino acids in length. Accordingly, the Gly/Ser linker consists of glycine and/or serine residues. In some embodiments, the intra-binding domain Gly/Ser liner is a peptide having the formula (Xaal)n wherein each amino acid residue Xaal is selected independently from glycine and serine, and n is an integer from 3 to 8. In some embodiments, the Gly/Ser intra-binding domain linker comprises the amino acid sequence of SEQ ID NO: 67 (GSTSGSGKPGSGEGSTKG) or 68 (GGGGS). In some embodiments, the intra-binding domain linker is a peptide having the amino acid formula [GGGGS (SEQ ID NO: 68)]ₘ, wherein m is an integer from 1 to 10, from 2 to 8, or from 3 to 5. In some embodiments, m is 5. In some embodiments, the inter-binding domain linker comprises the amino acid sequence of SEQ ID NO: 69 (GGGGSGGGGSGGGGSGGGGSGGGGS). In some aspects, an intra-binding domain linker can comprise a sequence of any of SEQ ID NOs: 61-66.

The first group of nucleic acid sequences 524 is joined to the second group of nucleic acid sequences 528 via a cleavable linker nucleotide sequence 526 that encodes a cleavable linker peptide. The cleavable linker peptide can be any suitable cleavable linker peptide. In some embodiments, the cleavable linker peptide is a self-cleaving linker peptide. Self-cleaving linker peptides include the 2A linker family, which includes T2A (EGRGSLLTCGDVEENPGP; SEQ ID NO: 75), P2A (ATNFSLLKQAGDVEENPGP; SEQ ID NO: 76), E2A (QCTNYALLKLAGDVESNPGP; SEQ ID NO: 77) and F2A (VKQTLNFDLLKLAGDVESNPGP; SEQ ID NO: 78). In some embodiments, the cleavable linker peptide is a furin-cleavable linker. Self-cleaving linker peptides include the GSG-2A linker family, including sequences put forth in SEQ ID NOs: 57-60.

In some embodiments, the intra-binding domain linker of the antigen binding domain of one or both CARs and/or the cleavable linker peptide can be optimized to maximize expression of the pair of CARs from the bicistronic CAR expression construct.

In certain embodiments, the antigen binding domain of one or both CARs comprise a leader sequence or signal peptide. Appropriate leader and signal peptides are fully described elsewhere herein.

In certain embodiments, TM domain 520 of the first CAR and TM 520′ of the second CAR are the same as those TM domains described elsewhere herein (i.e., a human CD8a TM domain; a CD28 TM domain).

In some embodiments, the first CAR and/or the second CAR comprises a hinge domain. In some embodiments, the hinge domain is a CD8a hinge domain. In particular embodiments, the hinge domain is a human CD8a hinge domain. In some embodiments, the CD8 hinge domain is, comprises, consists of, or consists essentially of a peptide having the amino acid sequence of SEQ ID NO: 72 (TTTPAPRPPTPAPTIASQPLSLRPEACRPAAGGAVHTRGLDFACD). In some embodiments, the hinge domain is a CD28 hinge domain. In particular embodiments, the hinge domain is a human CD28 hinge domain. In some embodiments, the CD28 hinge domain comprises, consists of, or consists essentially of a peptide having the amino acid sequence of SEQ ID NO: 80 (TSAAAIEVMYPPPYLDNEKSNGTIIHVKGKHLCPSPLFPGPSKP). In an embodiment, the CD28 TM domain is a peptide having the amino acid sequence of SEQ ID NO: 80.

In some embodiments, the hinge domain joins the antigen binding domain of a CAR to the transmembrane domain. In those embodiments that do not include a hinge domain, the antigen binding domain is joined to the transmembrane domain via a suitable linker.

In some embodiments, the first and/or second CAR comprises one or more spacer peptides. The spacer may between any aforementioned domain or region of the CAR. In an embodiment, the CAR comprises an IgG heavy chain constant domain (CH2CH3) spacer. In another embodiment, the spacer is between the scFv and the transmembrane domain.

In some embodiments, the intracellular T-cell signaling domains 522 and 522′ are the same as those described elsewhere herein (i.e., a 4-1BB intracellular T-cell signaling sequence or CD28 intracellular T-cell signaling sequence, and a CDζ intracellular T-cell signaling sequence). Embodiments described herein provide bicistronic CAR expression constructs encoding a pair of CARs.

An intracellular T-cell signaling domain of each of a first CAR and a second CAR can comprise any number and any combination of costimulatory domains. Costimulatory domains include, but not limited to, 4-1BB costimulatory domains, CD28 costimulatory domains, ICOS stimulatory domains, OX-40 costimulatory domains, CD40L costimulatory domains, CD27 costimulatory domains and TLR2 costimulatory domains. Sequences of the preceding costimulatory domains are known in the art. When more than one costimulatory domain is present in a single T-cell signaling domain, the costimulatory domains can be present in any order.

In some aspects, an intracellular T-cell signaling domain of each of a first CAR and a second CAR can comprise a CD28 costimulatory domain. In some aspects, An intracellular T-cell signaling domain of each of a first CAR and a second CAR can comprise a 4-1BB costimulatory domain. In some aspects, an intracellular T-cell signaling domain of each of a first CAR and a second CAR can comprise a CD28 costimulatory domain and a 4-1BB costimulatory domain. In some aspects, An intracellular T-cell signaling domain of each of a first CAR and a second CAR can comprise a CD28 costimulatory domain and an OX-40 costimulatory domain.

In some aspects, an intracellular T-cell signaling domain of each of a first CAR and a second CAR can comprise a CD3ζ intracellular T-cell signaling sequence. In some aspects, An intracellular T-cell signaling domain of each of a first CAR and a second CAR can comprise a CD3ζ intracellular T-cell signaling sequence and a CD28 costimulatory domain. In some aspects, an intracellular T-cell signaling domain of each of a first CAR and a second CAR can comprise a CD3ζ intracellular T-cell signaling sequence and a 4-1BB costimulatory domain. In some aspects, an intracellular T-cell signaling domain of each of a first CAR and a second CAR can comprise a CD3ζ intracellular T-cell signaling sequence, a CD28 costimulatory domain and a 4-1BB costimulatory domain. In some aspects, an intracellular T-cell signaling domain of each of a first CAR and a second CAR can comprise a CD3ζ intracellular T-cell signaling sequence, a CD28 costimulatory domain and an OX-40 costimulatory domain.

In some aspects of the bicistronic CAR constructs of the present disclosure, the intracellular T-cell signaling domain of a first CAR can be the same as the intracellular T-cell signaling domain of a second CAR.

In some aspects of the bicistronic CAR constructs of the present disclosure, the intracellular T-cell signaling domain of a first CAR can be distinct from the intracellular T-cell signaling domain of a second CAR.

In some aspects, the intracellular T-cell signaling domain of the first CAR can comprise a CD3ζ intracellular T-cell signaling sequence and the intracellular T-cell signaling domain of the second CAR can comprise any one of a CD3ζ intracellular T-cell signaling sequence and a CD28 costimulatory domain; a CD3ζ intracellular T-cell signaling sequence and a 4-1BB costimulatory domain; a CD3ζ intracellular T-cell signaling sequence, a CD28 costimulatory domain and a 4-1BB costimulatory domain; or a CD3ζ intracellular T-cell signaling sequence, a CD28 costimulatory domain and an OX-40 costimulatory domain. In some aspects, the intracellular T-cell signaling domain of the second CAR can comprise a CD3ζ intracellular T-cell signaling sequence and the intracellular T-cell signaling domain of the first CAR can comprise any one of a CD3ζ intracellular T-cell signaling sequence and a CD28 costimulatory domain; a CD3ζ intracellular T-cell signaling sequence and a 4-1BB costimulatory domain; a CD3ζ intracellular T-cell signaling sequence, a CD28 costimulatory domain and a 4-1BB costimulatory domain; or a CD3ζ intracellular T-cell signaling sequence, a CD28 costimulatory domain and an OX-40 costimulatory domain.

In some aspects, the intracellular T-cell signaling domain of the first CAR can comprise a CD3ζ intracellular T-cell signaling sequence and a CD28 costimulatory domain and the intracellular T-cell signaling domain of the second CAR can comprise any one of: a CD3ζ intracellular T-cell signaling sequence; a CD3ζ intracellular T-cell signaling sequence and a 4-1BB costimulatory domain; a CD3ζ intracellular T-cell signaling sequence, a CD28 costimulatory domain and a 4-1BB costimulatory domain; or a CD3ζ intracellular T-cell signaling sequence, a CD28 costimulatory domain and an OX-40 costimulatory domain. In some aspects, the intracellular T-cell signaling domain of the second CAR can comprise a CD3ζ intracellular T-cell signaling sequence and a CD28 costimulatory domain and the intracellular T-cell signaling domain of the first CAR can comprise any one of: a CD3ζ intracellular T-cell signaling sequence; a CD3ζ intracellular T-cell signaling sequence and a 4-1BB costimulatory domain; a CD3ζ intracellular T-cell signaling sequence, a CD28 costimulatory domain and a 4-1BB costimulatory domain; or a CD3ζ intracellular T-cell signaling sequence, a CD28 costimulatory domain and an OX-40 costimulatory domain.

In some aspects, the intracellular T-cell signaling domain of the first CAR can comprise a CD3ζ intracellular T-cell signaling sequence and a 4-1BB costimulatory domain and the intracellular T-cell signaling domain of the second CAR can comprise any one of: a CD3ζ intracellular T-cell signaling sequence and a CD28 costimulatory domain; a CD3ζ intracellular T-cell signaling sequence; a CD3ζ intracellular T-cell signaling sequence, a CD28 costimulatory domain and a 4-1BB costimulatory domain; or a CD3ζ intracellular T-cell signaling sequence, a CD28 costimulatory domain and an OX-40 costimulatory domain. In some aspects, the intracellular T-cell signaling domain of the second CAR can comprise a CD3ζ intracellular T-cell signaling sequence and a 4-1BB costimulatory domain and the intracellular T-cell signaling domain of the first CAR can comprise any one of: a CD3ζ intracellular T-cell signaling sequence and a CD28 costimulatory domain; a CD3ζ intracellular T-cell signaling sequence; a CD3ζ intracellular T-cell signaling sequence, a CD28 costimulatory domain and a 4-1BB costimulatory domain; or a CD3ζ intracellular T-cell signaling sequence, a CD28 costimulatory domain and an OX-40 costimulatory domain.

In some aspects, the intracellular T-cell signaling domain of the first CAR can comprise a CD3ζ intracellular T-cell signaling sequence, a CD28 costimulatory domain and a 4-1BB costimulatory domain and the intracellular T-cell signaling domain of the second CAR can comprise any one of: a CD3ζ intracellular T-cell signaling sequence and a CD28 costimulatory domain; a CD3ζ intracellular T-cell signaling sequence and a 4-1BB costimulatory domain; a CD3ζ intracellular T-cell signaling sequence; or a CD3ζ intracellular T-cell signaling sequence, a CD28 costimulatory domain and an OX-40 costimulatory domain. In some aspects, the intracellular T-cell signaling domain of the second CAR can comprise a CD3ζ intracellular T-cell signaling sequence, a CD28 costimulatory domain and a 4-1BB costimulatory domain and the intracellular T-cell signaling domain of the first CAR can comprise any one of a CD3ζ intracellular T-cell signaling sequence and a CD28 costimulatory domain; a CD3ζ intracellular T-cell signaling sequence and a 4-1BB costimulatory domain; a CD3ζ intracellular T-cell signaling sequence; or a CD3ζ intracellular T-cell signaling sequence, a CD28 costimulatory domain and an OX-40 costimulatory domain.

In some aspects, the intracellular T-cell signaling domain of the first CAR can comprise a CD3ζ intracellular T-cell signaling sequence, a CD28 costimulatory domain and an OX-40 costimulatory domain and the intracellular T-cell signaling domain of the second CAR can comprise any one of: a CD3ζ intracellular T-cell signaling sequence and a CD28 costimulatory domain; a CD3ζ intracellular T-cell signaling sequence and a 4-1BB costimulatory domain; a CD3ζ intracellular T-cell signaling sequence, a CD28 costimulatory domain and a 4-1BB costimulatory domain; or a CD3ζ intracellular T-cell signaling sequence. In some aspects, the intracellular T-cell signaling domain of the second CAR can comprise a CD3ζ intracellular T-cell signaling sequence, a CD28 costimulatory domain and an OX-40 costimulatory domain and the intracellular T-cell signaling domain of the first CAR can comprise any one of: a CD3ζ intracellular T-cell signaling sequence and a CD28 costimulatory domain; a CD3ζ intracellular T-cell signaling sequence and a 4-1BB costimulatory domain; a CD3ζ intracellular T-cell signaling sequence, a CD28 costimulatory domain and a 4-1BB costimulatory domain; or a CD3ζ intracellular T-cell signaling sequence.

Also provided are pairs of CARs expressed from a bicistronic CAR expression construct of the present disclosure. Accordingly, provide herein is a pair of CARs comprising a first CAR comprising an anti-FLT3 antigen binding domain, a transmembrane domain, and an intracellular T-cell signaling domain; and a second CAR comprising an antigen binding domain selected from the group consisting of an anti-CD19 antigen binding domain; an anti-CD22 antigen binding domain; an anti-CD33 antigen binding domain; and an anti-CD123 antigen binding domain, a transmembrane domain, and an intracellular T-cell signaling domain.

The present disclosure contemplates functional portions of the bivalent and bicistronic CARs disclosed and described herein. The term “functional portion,” when used in reference to a CAR, refers to any part or fragment of the CAR that retains the biological activity of the CAR from which it is derived (the parent CAR). Functional portions encompass, for example, those parts of a CAR that retain the ability to recognize target cells, or detect, treat, or prevent a disease or disorder (e.g., cancer), to a similar extent, the same extent, or to a higher extent, as the parent CAR In reference to the parent CAR the functional portion can comprise, for instance, about 90%, 95%, or more, of the parent CAR.

The functional portion can comprise additional amino acids at the amino or carboxy terminus of the functional portion, or at both termini, that are not found in the amino acid sequence of the parent CAR In some embodiments, the additional amino acids do not interfere with the biological function of the functional portion, e.g., recognize target cells, detect, treat or prevent a disease or disorder, etc. In particular embodiments, the additional amino acids enhance the biological activity of the functional portion, as compared to the biological activity of the parent CAR

The present disclosure also contemplates functional variants of the bivalent and bicistronic CARs disclosed and described herein. The term “functional variant” as used herein refers to a CAR, polypeptide, or protein having substantial or significant sequence identity or similarity to a parent CAR where the functional variant retains the biological activity of the parent CAR of which it is a variant. Functional variants encompass, for example, those variants of the CAR described herein (the parent CAR, either bivalent or bicistronic) that retain the ability to recognize target cells to a similar extent, the same extent, or to a higher extent, as the parent CAR In reference to the parent CAR the functional variant can, for instance, be at least about 90%, about 91%, about 92%, about 93%, about 94%, about 95%, about 96%, about 97%, about 98%, about 99% or more identical in amino acid sequence to the parent CAR.

A functional variant can, for example, comprise the amino acid sequence of the parent CAR with at least one conservative amino acid substitution. Alternatively, or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

The bivalent and bicistronic CARs of the embodiments of the present disclosure (including functional portions and functional variants of the disclosure) can comprise synthetic amino acids in place of one or more naturally-occurring amino acids. Such synthetic amino acids include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, omithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,β-diaminopropionic acid, homophenylalanine, and α-tert-butylglycine.

The bivalent and bicistronic CARs of the present disclosure (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The bivalent and bicistronic CARs of the present disclosure (including functional portions and functional variants thereof) can be obtained by any suitable method of making polypeptides or proteins, including de novo synthesis. Also, the CARs can be recombinantly produced using the nucleic acids described herein using standard recombinant methods. See, for instance, Green et al., Molecular Cloning: A Laboratory Manual, 4th ed., Cold Spring Harbor Press, Cold Spring Harbor, N.Y. 2012. Further, portions of some of the CARs described herein (including functional portions and functional variants thereof) can be isolated and/or purified from a source, such as a plant, a bacterium, an insect, a mammal, e.g., a rat, a human, etc. Alternatively, the CARs described herein (including functional portions and functional variants thereof) can be synthetic, recombinant, isolated, and/or purified.

Also provided herein is a nucleic acid molecule comprising a nucleotide sequence encoding any of the CARs described herein (including functional portions and functional variants thereof). In certain embodiments, the nucleic acid comprises a nucleotide sequence encoding any of the leader sequences, antigen binding domains, transmembrane domains, linkers, and/or intracellular T-cell signaling domains described herein.

In certain embodiments, the nucleic acid comprises a nucleotide sequence that encodes: an anti-FLT3 antigen binding domain; a second antigen binding domain selected from an anti-CD19 binding domain, an anti-CD22 antigen binding domain, an anti-CD33 binding domain and an anti-CD123 binding domain; a CD8 transmembrane domain; a 4-1BB intracellular T-cell signaling domain; and a CD3ζ intracellular T-cell signaling domain.

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions. In some embodiments, the nucleic acid may encode additional amino acid sequences that do not affect the function of the CAR and which may or may not be translated upon expression of the nucleic acid by a host cell.

In certain embodiments, the nucleic acid molecules disclosed herein are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

A recombinant nucleic acid molecule may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Green et al., supra. The nucleic acids can be constructed based on chemical synthesis and/or enzymatic ligation reactions using procedures known in the art. See, for example, Green et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine.

The nucleic acid molecule can comprise any isolated or purified nucleotide sequence which encodes any of the bivalent or bicistronic CARs disclosed and described herein, or functional portions or functional variants thereof.

An embodiment of the disclosure provides an isolated or purified nucleic acid molecule comprising a nucleotide sequence which is complementary to the nucleotide sequence of any of the nucleic acid molecules described herein or a nucleotide sequence which hybridizes under stringent conditions to the nucleotide sequence of any of the nucleic acids described herein.

The nucleotide sequence which hybridizes under stringent conditions may hybridize under high stringency conditions. “High stringency conditions” means that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. An example of high stringency conditions includes low salt and/or high temperature conditions, such as provided by about 0.02-0.1 M NaCl or the equivalent, at temperatures of about 50-70° C. Such conditions tolerate little, if any, mismatch between the nucleotide sequence and the template or target strand and are particularly suitable for detecting expression of any of the inventive CARs. It is generally appreciated that conditions can be rendered more stringent by the addition of increasing amounts of formamide.

This disclosure also provides a nucleic acid comprising a nucleotide sequence that is at least about 90% or more, e.g., about 91%, about 92%, about 93%, about 94%, about 95%, about 96%, about 97%, about 98%, or about 99% identical to any of the nucleic acids described herein.

In certain embodiments, the nucleic acid molecules of the present disclosure and expression constructs comprising the nucleic acid molecules of the present disclosure can be incorporated into a recombinant expression vector. In this regard, some embodiments provide recombinant expression vectors comprising any of the nucleic acid molecules or expression constructs of the disclosure. The term “recombinant expression vector” refers to a genetically-modified oligonucleotide or polynucleotide construct that permits the expression of an mRNA, protein, polypeptide, or peptide by a host cell, when the construct comprises a nucleotide sequence or expression construct encoding the mRNA, protein, polypeptide, or peptide, and the vector is contacted with the host cell under conditions sufficient to have the mRNA, protein, polypeptide, or peptide expressed within the cell. The vectors of the present disclosure are not naturally-occurring as a whole. However, parts of the vectors can be naturally-occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. In some embodiments, the non-naturally occurring or altered nucleotides or internucleotide linkages do not hinder the transcription or replication of the vector.

In certain embodiments, the recombinant expression vectors described herein comprise a suitable recombinant expression vector backbone and can be used to transform or transfect any suitable host cell. Suitable vector backbones include those designed for propagation and expansion or for expression or both, such as plasmids and viruses. The vector backbones can be selected from the group of the pUC vector series (Fermentas Life Sciences, Glen Burnie, Md.), the pBluescript vector series (Stratagene, LaJolla, Calif.), the pET vector series (Novagen, Madison, Wis.), the pGEX vector series (Pharmacia Biotech, Uppsala, Sweden), and the pEX vector series (Clontech, Palo Alto, Calif.). Bacteriophage vector backbones, such as λGT10, λGTll, λZapll (Stratagene), λEMBL4, and λNMll49, also can be used. Examples of animal expression vectors include pEUK-Cl, pMAM, and pMAMneo (Clontech). The recombinant expression vector may be a viral vector, e.g., a retroviral vector or a lentiviral vector.

In some embodiments, the recombinant expression vectors described herein can be prepared using the recombinant DNA techniques described in, for example, Green et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may also comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G4 1 8 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CAR (including functional portions and functional variants thereof), or to the nucleotide sequence which is complementary to or which hybridizes to the nucleotide sequence encoding the CAR. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Included in the scope of the present disclosure are conjugates, e.g., bioconjugates, comprising any of the inventive CARs (including any of the functional portions or variants thereof), nucleic acids, recombinant expression vectors, host cells, or populations of hostcells.

An embodiment of the disclosure further provides a host cell comprising any of the recombinant expression vectors described herein. As used herein, the term “host cell” refers to any type of cell that can contain and express a recombinant expression vector described herein. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells include, for example, DH5α E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5α cell. For purposes of producing a recombinant CAR, the host cell can be a mammalian cell. The host cell can be a human cell. While the host cell can be of any cell type, can originate from any type of tissue, and can be of any developmental stage, the host cell can be a peripheral blood lymphocyte (PBL) or a peripheral blood mononuclear cell (PBMC). The host cell can be a T-cell.

The T-cell can be any T-cell, such as a cultured T-cell, e.g., a primary T-cell, or a T-cell from a cultured T-cell line, e.g., Jurkat, SupTl, etc., or a T-cell obtained from a mammal. If obtained from a mammal, the T-cell can be obtained from numerous sources, including but not limited to blood, bone marrow, lymph node, the thymus, or other tissues or fluids. T-cells can also be enriched for or purified. The T-cell can be a human T-cell. The T-cell can be a T-cell isolated from a human. The T-cell can be any type of T-cell and can be of any developmental stage, including but not limited to, CD4+/CD8+ double positive T-cells, CD4+ helper T-cells, e.g., Th1 and Th2 cells, CD8+ T-cells (e.g., cytotoxic T-cells), tumor infiltrating cells, memory T-cells, naive T-cells, and the like. The T-cell may be a CD8+ T-cell or a CD4+ T-cell.

Also provided herein is a population of cells comprising at least one host cell described herein. The population of cells can be a heterogeneous population comprising the host cell comprising any of the recombinant expression vectors described, in addition to at least one other cell, e.g., a host cell (e.g., a T-cell), which does not comprise any of the recombinant expression vectors, or a cell other than a T-cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the disclosure, the population of cells is a clonal population comprising host cells comprising a recombinant expression vector as described herein.

The bivalent and bicistronic CARs (including functional portions and variants thereof), nucleic acids, recombinant expression vectors, and host cells (including populations thereof) described herein (collectively, “CAR materials”) can be isolated and/or purified. The term “isolated” as used herein means having been removed from its natural environment. The term “purified” or “isolated” does not require absolute purity or isolation; rather, it is intended as a relative term. Thus, for example, a purified (or isolated) host cell preparation is one in which the host cell is more pure than cells in their natural environment within the body. In some embodiments, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

[The CAR materials can be formulated into a composition, such as a pharmaceutical composition. In this regard, an embodiment of the disclosure provides a pharmaceutical composition comprising any of the CAR materials described herein, and a pharmaceutically acceptable carrier. The pharmaceutical compositions containing any of the CAR materials can comprise more than one CAR material, e.g., a CAR and a nucleic acid molecule, or two or more different CARs. Alternatively, the pharmaceutical composition can comprise a CAR material in combination with other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In a particular embodiment, the pharmaceutical composition comprises a host cell expressing a bivalent or bicistronic CAR of the present disclosure, or populations thereof.

A pharmaceutically acceptable carrier can be any of those conventionally used and is limited only by chemico-physical considerations, such as solubility and lack of reactivity with the active agent(s), and by the route of administration. The pharmaceutically acceptable carriers described herein include, for example, vehicles, adjuvants, excipients, and diluents. It is preferred that the pharmaceutically acceptable carrier be one which has no detrimental side effects or toxicity under the conditions of use.

The choice of carrier will be determined in part by the particular CAR material to be included, as well as by the particular method used to administer the CAR material. Accordingly, there are a variety of suitable formulations of the pharmaceutical composition of the present disclosure.

The CAR materials may be administered in any suitable manner. In some embodiments, the CAR materials are administered by injection, (e.g., subcutaneously, intravenously, intratumorally, intraarterially, intramuscularly, intradermally, interperitoneally, or intrathecally). In particular embodiments, the CAR materials are administered intravenously. A suitable pharmaceutically acceptable carrier for the CAR material for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, Ill.), PLASMA-LYTE A (Baxter, Deerfield, Ill.), about 5% dextrose in water, or Ringer's lactate. In an embodiment, the pharmaceutically acceptable carrier is supplemented with human serum albumen.

An “effective amount” or “an amount effective to treat” refers to a dose that is adequate to prevent or treat ALL, AML or another cancer in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the CAR material selected, method of administration, timing and frequency of administration, the existence, nature, and extent of any adverse side-effects that might accompany the administration of a particular CAR material, and the desired physiological effect. It will be appreciated by one of skill in the art that various diseases or disorders could require prolonged treatment involving multiple administrations, perhaps using the CAR materials in each or various rounds of administration. By way of example and not intending to be limiting, when the CAR material is a host cell expressing a CAR, an exemplary dose of host cells may be a minimum of one million cells (1×106 cells/dose).

The amount or dose of the CAR material administered should be sufficient to affect a therapeutic or prophylactic response in the subject or animal over a reasonable time frame. For example, the dose of the CAR material should be sufficient to bind to antigen, or detect, treat or prevent cancer in a period of from about 2 hours or longer, e.g., about 12 to about 24 or more hours, from the time of administration. In certain embodiments, the time period could be even longer. The dose will be determined by the efficacy of the particular inventive CAR material and the condition of the animal (e.g., human), as well as the body weight of the animal (e.g., human) to be treated.

When the CAR materials are administered with one or more additional therapeutic agents, one or more additional therapeutic agents can be co-administered to the mammal. “Co-administer” and derivatives thereof mean administering one or more additional therapeutic agents and the CAR materials sufficiently close in time such that the CAR materials can enhance the effect of one or more additional therapeutic agents, or vice versa. In this regard, the CAR materials can be administered first, and the one or more additional therapeutic agents can be administered second, or vice versa. Alternatively, the CAR materials and the one or more additional therapeutic agents can be administered simultaneously. An exemplary therapeutic agent that can be co-administered with the CAR materials is IL-2.

It is contemplated that the CAR materials can be used in methods of treating or preventing a disease in a mammal. Without being bound to a particular theory or mechanism, the CAR materials have biological activity, e.g., ability to recognize antigen, e.g., FLT3 and one of CD19, CD22, CD33 and CD123, such that the CAR when expressed by a host cell is able to mediate an immune response against the cell expressing the antigen, e.g., FLT3 and one of CD19, CD22, CD33 and CD123, as the CAR has dual specificity. In this regard, an embodiment provides a method of treating or preventing ALL, AML and/or another cancer in a mammal, comprising administering to the mammal any of the CARs, the nucleic acids, the recombinant expression vectors, the host cells, the population of cells, and/or the pharmaceutical compositions of the disclosure in an amount effective to treat or prevent ALL, AML and/or another cancer in the mammal.

Wherein host cells or populations of cells are administered, the cells can be cells that are allogeneic or autologous to the mammal. In particular embodiments, the cells are autologous to the mammal.

The mammal referred to herein can be any mammal. In particular embodiments, the mammal is a human.

The cancer to be treated by the methods described herein can be any cancer characterized by surface expression of FLT3 and CD19, FLT3 and CD22, FLT3 and CD 33 or FLT3 and CD123. Cancers characterized by surface expression of FLT3 and CD19 include, but are not limited to, acute lymphoblastic leukemia (ALL) and B-cell acute lymphoblastic leukemia (B-ALL). Cancers characterized by surface expression of FLT3 and CD22 include but are not limited to ALL and B-ALL. Cancers characterized by surface expression of FLT3 and CD33 includes acute myeloid leukemia (AML). In some embodiments, the cancer is a relapsed or refractory cancer. In some embodiments, the cancer has relapsed due to a loss of or reduction in surface expression of FLT3 or CD19, of FLT3 or CD22, or of FLT3 or CD33. In some embodiments, the cancer is refractory due to a lack of surface expression or minimal surface expression of FLT3 or CD19, of FLT3 or CD22, of FLT3 or CD33, or of FLT3 and CD123.

The terms “treat,” and “prevent” as well as words stemming therefrom, as used herein, do not necessarily imply 100% or complete treatment or prevention. Rather, there are varying degrees of treatment or prevention that may be recognized as having a potential benefit or therapeutic effect. In this respect, the methods provided herein can provide any amount of any level of treatment or prevention of cancer in a mammal. Furthermore, the treatment or prevention provided by the described methods can include treatment or prevention of one or more conditions or symptoms of the disease, e.g., cancer, being treated or prevented. Also, for purposes herein, “prevention” can encompass delaying the onset of the disease, or a symptom or condition thereof.

Another embodiment provides a use of the inventive CARs, nucleic acids, recombinant expression vectors, host cells, populations of cells, or pharmaceutical compositions, for the treatment or prevention of ALL, AML and/or another cancer in a mammal.

Another embodiment provides a method of detecting the presence of ALL, AML and/or another cancer in a mammal, comprising: contacting a sample comprising one or more cells from the mammal with the CARs or the present disclosure, thereby forming a complex, and detecting the complex, wherein detection of the complex is indicative of the presence of ALL, AML and/or another cancer in the mammal.

Detection of the complex can occur through any number of ways. For example, the CARs can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

Table 9 contains a summary of the sequences referenced herein.

TABLE 9
Summary Table of Referenced Sequences

| | | SEQ ID |
|---|---|---|
| Description | Sequence | NO |
| NC7 scFv V_{H}- | EVQLVQSGAEVKKPGSSVKVSCKAS | 1 |
| FR1 | | |
| NC7 scFv V_{H}- | GGTFSSYAIS | 2 |
| CDR1 | | |
| NC7 scFv V_{H}- | WVRQAPGQGLEWMG | 3 |
| FR2 | | |
| NC7 scFv V_{H}- | GIIPIFGTANYAQKFQG | 4 |
| CDR2 | | |
| NC7 scFv V_{H}- | RVTITADKSTSTAYMELSSLRSEDTAVYYCAT | 5 |
| FR3 | | |
| NC7 scFv V_{H}- | FALFGFREQAFDI | 6 |
| CDR3 | | |
| NC7 scFv V_{H}- | WGQGTTVTVSS | 7 |
| FR4 | | |
| NC7 scFv Linker | GGGGSGGGGSGGGGS | 8 |
| NC7 scFv V_{L}- | DIQMTQSPSSLSASVGDRVTITC | 9 |
| FR1 | | |
| NC7 scFv V_{L}- | RASQSISSYLN | 10 |
| CDR1 | | |
| NC7 scFv V_{L}- | WYQQKPGKAPKLLIY | 11 |
| FR2 | | |
| NC7 scFv V_{L}- | AASSLQS | 12 |
| CDR2 | | |
| NC7 scFv V_{L}- | GVPSRFSGSGSGTDFTLTISSLQPEDLATYYC | 13 |
| FR3 | | |
| NC7 scFv V_{L}- | QQSYSTPFT | 14 |
| CDR3 | | |
| NC7 scFv V_{L}- | FGPGTKVDIK | 15 |
| FR4 | | |
| NC7 scFv V_{H} | EVQLVQSGAEVKKPGSSVKVSCKASGGTFSSYAISWVRQAP | 16 |
| (CDRs: | GQGLEWMGGIIPIFGTANYAQKFQGRVTITADKSTSTAYM | |
| bold and | ELSSLRSEDTAVYYCATFALFGFREQAFDIWGQGTTVTVSS | |
| underlined) | | |
| NC7 scFv V_{L} | DIQMTQSPSSLSASVGDRVTITCRASQSISSYLNWYQQKPGK | 17 |
| (CDRs: | APKLLIYAASSLQSGVPSRFSGSGSGTDFTLTISSLQPEDLAT | |
| bold and | YYCQQSYSTPFTFGPGTKVDIK | |
| underlined) | | |
| NC7 scFv | EVQLVQSGAEVKKPGSSVKVSCKASGGTFSSYAISWVRQAP | 18 |
| | GQGLEWMGGIIPIFGTANYAQKFQGRVTITADKSTSTAYME | |
| | LSSLRSEDTAVYYCATFALFGFREQAFDIWGQGTTVTVSSG | |
| | GGGSGGGGSGGGGSDIQMTQSPSSLSASVGDRVTITCRASQS | |
| | ISSYLNWYQQKPGKAPKLLIYAASSLQSGVPSRFSGSGSGTD | |
| | FTLTISSLQPEDLATYYCQQSYSTPFTFGPGTKVDIK | |
| fmC63 scFv V_{H}- | EVKLQESGPGLVAPSQSLSVTCTVS | 19 |
| FR1 | | |
| fmC63 scFv V_{H}- | GVSLPDYG | 20 |
| CDR1 | | |
| fmC63 scFv V_{H}- | VSWIRQPPRKGLEWLGV | 21 |
| FR2 | | |
| fmC63 scFv V_{H}- | IWGSETT | 22 |
| CDR2 | | |
| fmC63 scFv V_{H}- | YYNSALKSRLTIIKDNSKSQVFLKMNSLQTDDTAIYYC | 23 |
| FR3 | | |
| fmC63 scFv V_{H}- | AKHYYYGGSYAMDY | 24 |
| CDR3 | | |
| fmC63 scFv V_{H}- | WGQGTSVTVSS | 25 |
| FR4 | | |
| fmC63 scFv V_{L}- | DIQMTQTTSSLSASLGDRVTISCRAS | 26 |
| FR1 | | |
| fmC63 scFv V_{L}- | QDISKY | 27 |
| CDR1 | | |
| fmC63 scFv V_{L}- | LNWYQQKPDGTVKLLIY | 28 |
| FR2 | | |
| fmC63 scFv V_{L}- | HTS | 29 |
| CDR2 | | |
| fmC63 scFv V_{L}- | RLHSGVPSRFSGSGSGTDYSLTISNLEQEDIATYFC | 30 |
| FR3 | | |
| fmC63 scFv V_{L}- | QQGNTLPYT | 31 |
| CDR3 | | |
| fmC63 scFv V_{L}- | FGGGTKLEIT | 32 |
| FR4 | | |
| fmC63 scFv V_{H} | EVKLQESGPGLVAPSQSLSVTCTVSGVSLPDYGVSWIRQPPR | 33 |
| | KGLEWLGVIWGSETTYYNSALKSRLTIIKDNSKSQVFLKMN | |
| | SLQTDDTAIYYCAKHYYYGGSYAMDYWGQGTSVTVSS | |
| fmC63 scFv V_{L} | DIQMTQTTSSLSASLGDRVTISCRASQDISKYLNWYQQKPDG | 34 |
| | TVKLLIYHTSRLHSGVPSRFSGSGSGTDYSLTISNLEQEDIAT | |
| | YFCQQGNTLPYTFGGGTKLEIT | |
| m971 scFv V_{H}- | QVQLQQSGPGLVKPSQTLSLTCAIS | 35 |
| FR1 | | |
| m971 scFv V_{H}- | GDSVSSNSAA | 36 |
| CDR1 | | |
| m971 scFv V_{H}- | WNWIRQSPSRGLEWLGR | 37 |
| FR2 | | |
| m971 scFv V_{H}- | TYYRSKWYN | 38 |
| CDR2 | | |
| m971 scFv V_{H}- | NDYAVSVKSRITINPDTSKNQFSLQLNSVTPEDTAVYYC | 39 |
| FR3 | | |
| m971 scFv V_{H}- | AREVTGDLEDAFDI | 40 |
| CDR3 | | |
| m971 scFv V_{H}- | WGQGTMVTVSS | 41 |
| FR4 | | |
| m971 scFv V_{L}- | DIQMTQSPSSLSASVGDRVTITCRAS | 42 |
| FR1 | | |
| m971 scFv V_{L}- | QTIWSY | 43 |
| CDR1 | | |
| m971 scFv V_{L}- | LNWYQQRPGKAPNLLIY | 44 |
| FR2 | | |
| m971 scFv V_{L}- | AAS | 45 |
| CDR2 | | |
| m971 scFv V_{L}- | SLQSGVPSRFSGRGSGTDFTLTISSLQAEDFATYYC | 46 |
| FR3 | | |
| m971 scFv V_{L}- | QQSYSIPQT | 47 |
| CDR3 | | |
| m971 scFv V_{L}- | FGQGTKLEIK | 48 |
| FR4 | | |
| m971 scFv V_{H} | QVQLQQSGPGLVKPSQTLSLTCAISGDSVSSNSAAWNWIRQ | 49 |
| | SPSRGLEWLGRTYYRSKWYNDYAVSVKSR1TINPDTSKNQF | |
| | SLQLNSVTPEDTAVYYCAREVTGDLEDAFDIWGQGTMVTV | |
| | SS | |
| m971 scFv V_{L} | DIQMTQSPSSLSASVGDRVTITCRASQTIWSYLNWYQQRPG | 50 |
| | KAPNLLIYAASSLQSGVPSRFSGRGSGTDFTLTISSLQAEDFA | |
| | FYYCQQSYSIPQTFGQGTKLEIK | |
| HuM195 scFv | QVQLVQSGAEVKKPGSSVKVSCKASGYTFTDYNMHWVRQ | 51 |
| V_{H} (CDRs: | APGKGLEWIGYIYPYNGGTGYNKKFKSKATITADESTNTA | |
| bold and | YMELSSLRSEDTAVYYCARGRPAMDYWGKGTLVTVSS | |
| underlined) | | |
| HuM195 scFv | DIKMTKSPSSLSASVGDRVTITCRASESVDNYGISFMNWFK | 52 |
| V_{L} (CDRs: | KKPGKAPKLLIYAASNKGSGVPSRFSGSGSGTDFTLTISSLKP | |
| bold and | DDFATYYCKKSKEVPWTFGKGTKVEIK | |
| underlined) | | |
| M195 scFv | EVQLQQSGPELVKPGASVKISCKASGYTFTDYNMHWVKQS | 53 |
| V_{H} (CDRs: | HGKSLEWIGYIYPYNGGTGYNKKFKSKATLTVDNSSSTAY | |
| bold and | MDVRSLTSEDSAVYYCARGRPAMDYWGQGTSVTVSS | |
| underlined) | | |
| M195 scFv | DIVLTQSPASLAVSLGQRATISCRASESVDNYGISFMNWFQ | 54 |
| V_{L} (CDRs: | QKPGQPPKLLIYAASNKGSGVPARFSGSGSGTDFSLNIHPME | |
| bold and | EDDTAMYFCKKSKEVPWTFGGGTKLEIK | |
| underlined) | | |
| h-p67.6 scFv | MEWSWVFLFFLSVTTGVHSEVQLVQSGAEVKKPGSSVKVS | 55 |
| V_{H} | CKASGYTITDSNIHWVRQAPGQSLEWIGYIYPYNGGTDYNQ | |
| | KFKNRATLTVDNPTNTAYMELSSLRSEDTDFYYCVNGNPW | |
| | LAYWGQGTLVTVSSASTKGP | |
| h-p67.6 scFv | MSVPTQVLGLLLLWLTDARCDIQLTQSPSTLSASVGDRVTIT | 56 |
| V_{L} | CRASESLDNYGIRFLTWFQQKPGKAPKLLMYAASNQGSGVP | |
| | SRFSGSGSGTEFTLTISSLQPDDFATYYCQQTKEVPWSFGQG | |
| | FKVEVKRT | |
| GSG-T2A linker | GSGEGRGSLLTCGDVEENPGP | 57 |
| GSG-P2A linker | GSGATNFSILKQAGDVEENPGP | 58 |
| GSG-E2A linker | GSGQCTNYALLKLAGDVESNPGP | 59 |
| GSG-F2A linker | GSGVKQTLNFDLLKLAGDVESNPGP | 60 |
| Intra-binding | GGGGSGGGGS | 61 |
| domain linker | | |
| Intra-binding | GGGGSGGGGSGGGGS | 62 |
| domain linker | | |
| Intra-binding | GGGGSGGGGSGGGGSGGGGS | 63 |
| domain linker | | |
| Intra-binding | GGGGSGGGGSGGGGSGGGGSGGGGSGGGGS | 64 |
| domain linker | | |
| Intra-binding | GGGGSGGGGSGGGGSGGGGSGGGGSGGGGSGGGGS | 65 |
| domain linker | | |
| Intra-binding | GGGGSGGGGSGGGGSGGGGSGGGGSGGGGSGGGGSGGGG | 66 |
| domain linker | S | |
| Intra-binding | GSTSGSGKPGSGEGSTKG | 67 |
| domain linker | | |
| intra-binding | GGGGS | 68 |
| domain linker | | |
| Intra-binding | GGGGSGGGGSGGGGSGGGGSGGGGS | 69 |
| domain linker | | |
| Leader sequence | LLLVTSLLLCELPHPAFLLIP | 70 |
| HumanCD8a TM | IYIWAPLAGTCGVLLLSLVITLYC | 71 |
| domain | | |
| HumanCD8a | TTTPAPRPPTPAPTIASQPLSLRPEACRPAAGGAVHTRGLDFA | 72 |
| hinge | CD | |
| 4-1BB | KRGRKKLLYIFKQPFMRPVQTTQEEDGCSCRFPEEEEGGCEL | 73 |
| intracellular | | |
| signaling | | |
| sequence | | |
| CD3 ζ | RVKFSRSADAPAYXQGQNQLYNELNLGRREEYDVLDKRRG | 74 |
| intracellular T | RDPEMGGKPRRKNPQEGLYNELQKDKMAEAYSEIGMKGER | |
| cell signaling | RRGKGHDGLYQGLSTATKDTYDALHMQALPPR misc feature | |
| sequence | -(14)..(14)-Xaa at position 14 may be Lys or Gln | |
| T2A linker | EGRGSLLTCGDVEENPGP | 75 |
| P2A linker | ATNFSLLKQAGDVEENPGP | 76 |
| E2A linker | QCTNYALLKLAGDVESNPGP | 77 |
| F2A linker | VKQTLNFDLLKLAGDVESNPGP | 78 |
| HumanCD28 TM | FWVLVVVGGVLACYSLLVTVAFIIFWV | 79 |
| domain | | |
| HumanCD28 | TSAAAIEVMYPPPYLDNEKSNGTIIHVKGKHLCPSPLFPGPSK | 80 |
| hinge | P | |
| CD28 | KRGRKKLLYIFKQPFMRPVQTTQEEDGCSCRFPEEEEGGCEL | 81 |
| intracellular | | |
| signaling | | |
| sequence | | |
| M1006 scFv V_{H} | EVQLVESGGGLVQPGGSLRLSCAASGFTFSSYGMHWVRQA | 82 |
| | PGKGLEWVAVISYDGSNKYYADSVKGRFTISRDNSKNTLYL | |
| | QMNSLRAEDTAVYYCANLAPWAAYWGQGTLVTVSS | |
| M1006 scFv V_{L} | EIVLTQSPLSLPVTPGEPASISCRSSQSLLHSNGYNYLDWYLQ | 83 |
| | KPGQSPQLLIYLGSNRASGVPDRFSGSGSGTDFTLKISRVEAE | |
| | DVGVYYCMQALQTPHTFGQGTKLEIK | |
| M1006 scFv | EVQLVESGGGLVQPGGSLRLSCAASGFTFSSYGMHWVRQA | 84 |
| | PGKGLEWVAVISYDGSNKYYADSVKGRFTISRDNSKNTLYL | |
| | QMNSLRAEDTAVYYCANLAPWAAYWGQGTLVTVSSGGGG | |
| | SGGGGSGGGGSEIVLTQSPLSLPVTPGEPASISCRSSQSLLHS | |
| | NGYNYLDWYLQKPGQSPQLLIYLGSNRASGVPDRFSGSGSG | |
| | TDFTLKISRVEAEDVGVYYCMQALQTPHTFGQGTKLEIK | |
| M1007 scFv V_{H} | EVQLVESGGGVVQPGGSLRLSCAASGFTFSSYGMHWVRQA | 85 |
| | PGKGLEWVAVISYDGSNKYYADSVKGRFTISRDNSKNTLYL | |
| | QMNSLRAEDTAVYYCANLAPWAAYWGQGTLVTVSS | |
| M1007 scFv V_{L} | DVVMTQSPLSLPVTPGEPASISCRSSQSLLHSNGYNYLDWYL | 86 |
| | QKPGQSPQLLIYLGSNRASGVPDRFSGSGSGTDFTLKISRVEA | |
| | EDVGVYYCMQALQTPLTFGGGTKVEIK | |
| M1007 scFv | EVQLVESGGGVVQPGGSLRLSCAASGFTFSSYGMHWVRQA | 87 |
| | PGKGLEWVAVISYDGSNKYYADSVKGRFTISRDNSKNTLYL | |
| | QMNSLRAEDTAVYYCANLAPWAAYWGQGTLVTVSSGGGG | |
| | SGGGGSGGGGSDVVMTQSPLSLPVTPGEPASISCRSSQSLLH | |
| | SNGYNYLDWYLQKPGQSPQLLIYLGSNRASGVPDRFSGSGS | |
| | GTDFTLKISRVEAEDVGVYYCMQALQTPLTFGGGTKVEIK | |
| 26292 scFv V_{H} | QVQLQQPGAELVRPGASVKLSCKASGYTFTSYWMNWVKQ | 88 |
| (CDRs: | RPDQGLEWIGRIDPYDSETHYNQKFKDKAILTVDKSSSTAY | |
| bold and | MQLSSLTSEDSAVYYCARGNWDDYWGQGTTLTVSS | |
| underlined) | | |
| 26292 scFv V_{H}- | SYWMN | 89 |
| CDR1 | | |
| 26292 scFv V_{H}- | RIDPYDSETHYNQKFKD | 90 |
| CDR2 | | |
| 26292 scFv V_{H}- | GNWDDY | 91 |
| CDR3 | | |
| 26292 scFv V_{L} | DVQITQSPSYLAASPGETITINCRASKSISKDLAWYQEKPGK | 92 |
| (CDRs: | TNKLLIYSGSTLQSGIPSRFSGSGSGTDFTLTISSLEPEDFAMY | |
| bold and | YCQQHNKYPYTFGGGTKLEIK | |
| underlined) | | |
| 26292 scFv V_{L}- | RASKSISKDLA | 93 |
| CDR1 | | |
| 26292 scFv V_{L}- | SGSTLQS | 94 |
| CDR2 | | |
| 26292 scFv V_{L}- | QQHNKYPYT | 95 |
| CDR3 | | |
| 32701 scFv V_{H} | QIQLVQSGPELKKPGETVKISCKTSGYVFTNYGMNWVKQAP | 96 |
| (CDRs: | GKGFKWMGWMNTNTGEPTSLEDFKGRFAFSLETSASTAY | |
| bold and | LQINNLKNDDTATYFCARSGGYDPMDYWGQGTSVTVSS | |
| underlined) | | |
| 32701 scFv V_{H}- | NYGMN | 97 |
| CDR1 | | |
| 32701 scFv V_{H}- | WMNTNTGEPTSLEDFKG | 98 |
| CDR2 | | |
| 32701 scFv V_{H}- | SGGYDPMDY | 99 |
| CDR3 | | |
| 32701 scFv V_{L} | DIVLTQSPASLAVSPGQRATISCRASESVDNYGNTFMHWYQ | 100 |
| (CDRs: | QKPGQPPKLLIYRASNLESGIPARFSGSDSRTDFTLTINPVEA | |
| bold and | DDVATYYCQQSKEDPPTFGGTKLELK | |
| underlined) | | |
| 32701 scFv V_{L}- | RASESVDNYGNTFMH | 101 |
| CDR1 | | |
| 32701 scFv V_{L}- | RASNLES | 102 |
| CDR2 | | |
| 32701 scFv V_{L}- | QQSKEDPPT | 103 |
| CDR3 | | |
| 32716 V_{H} (CDRs: | QIQLVQSGPELKKPGETVKISCKASGYIFTNYGMNWVKQAP | 104 |
| bold and | GKSFKWMGWINTYTGESTYSADFKGRFAFSLETSASTAYL | |
| underlined) | HINDLKNEDTATYFCARSGGYDPMDYWGQGTSVTVSS | |
| 32716 V_{H}- | NYGMN | 105 |
| CDR1 | | |
| 32716 V_{H}- | WINTYTGESTYSADFKG | 106 |
| CDR2 | | |
| 32716 V_{H}- | SGGYDPMDY | 107 |
| CDR3 | | |
| 32716 V_{L} (CDRs: | DIVLTQSPASLAVSLGQRATISCRASESVDNYGNTFMHWYQ | 108 |
| bold and | QKPGQPPKLLIYRASNLESGIPARFSGSGSRTDFTLTINPVEA | |
| underlined) | DDVATYYCQQSNEDPPTFGAGTKELK | |
| 32716 V_{L}- | RASESVDNYGNTFMH | 109 |
| CDR1 | | |
| 32716 V_{L}- | RASNLES | 110 |
| CDR2 | | |
| 32716 V_{L}- | QQSNEDPPT | 111 |
| CDR3 | | |
| 32703V_{H} (CDRs: | QVQLQQPGAELVKPGAPVKLSCKASGYTFTNYWMNWIKQ | 112 |
| bold and | RPGRGLEWIGRIDPSDSESHYNQKFKDKATLTVDKSSNTAY | |
| underlined) | IQLSSLTSEDSAVYYCARYDYDDTMDYWGQGTSVTVSS | |
| 32703 V_{H}- | NYWMN | 113 |
| CDR1 | | |
| 32703 V_{H}- | RIDPSDSESHYNQKFKD | 114 |
| CDR2 | | |
| 32703 V_{H}- | YDYDDTMDY | 115 |
| CDR3 | | |
| 32703 V_{L} (CDRs: | DIVMTQAAPSVPVTPGESVSISCRSNKSLLHSNGNTYLYWF | 116 |
| bold and | LQRPGQSPQLLIYRMSNLASGVPDRFSGSGSGTAFTLRISRV | |
| underlined) | EAEDVGVYYCMQHLEYPYTFGGGTKLEIK | |
| 32703 V_{L}- | RSNKSLLHSNGNTYLY | 117 |
| CDR1 | | |
| 32703 V_{L}- | RMSNLAS | 118 |
| CDR2 | | |
| 32703 V_{L}- | MQHLEYPYT | 119 |
| CDR3 | | |

### EXEMPLARY EMBODIMENTS OF THE PRESENT DISCLOSURE

Embodiment 1. A nucleic acid molecule comprising:

a) a nucleic acid sequence encoding a first leader sequence;

b) a nucleic acid sequence encoding an scFv that binds to CD19;

c) a nucleic acid sequence encoding a CD28 hinge region;

d) a nucleic acid encoding a CD28 transmembrane domain;

e) a nucleic acid sequence encoding a CD28 intracellular signaling sequence;

f) a nucleic acid sequence encoding a CD3ζ intracellular T cell signaling sequence;

g) a nucleic acid sequence encoding a self-cleaving linker peptide;

h) a nucleic acid sequence encoding a second leader sequence;

i) a nucleic acid sequence encoding an scFv that binds to FLT3;

j) a nucleic acid sequence encoding a CD8a hinge domain;

k) a nucleic acid sequence encoding a CD8a transmembrane domain;

l) a nucleic acid sequence encoding a 4-1BB intracellular signaling sequence; and

m) a nucleic acid sequence encoding a CD3ζ intracellular T cell signaling sequence.

Embodiment 2. A polypeptide comprising:

a) a first leader sequence;

b) an scFv that binds to CD19;

c) a CD28 hinge region;

d) a CD28 transmembrane domain;

e) a CD28 intracellular signaling sequence;

f) a CD3ζ intracellular T cell signaling sequence;

g) a self-cleaving linker peptide;

h) a second leader sequence;

i) an scFv that binds to FLT3;

j) a CD8a hinge domain;

k) a CD8a transmembrane domain;

l) a 4-1BB intracellular signaling sequence; and

m) a CD3ζ intracellular T cell signaling sequence.

Embodiment 3. The polypeptide of embodiment 2, wherein the polypeptide comprises the amino acid sequence put forth in SEQ ID NO: 138.

Embodiment 4. A composition comprising a first polypeptide and a second polypeptide, wherein the first polypeptide comprises:
a) a first leader sequence;
b) an scFv that binds to CD19;
c) a CD28 hinge region;
d) a CD28 transmembrane domain;
e) a CD28 intracellular signaling sequence;
f) a CD3ζ intracellular T cell signaling sequence; and

wherein the second polypeptide comprises:
a) a second leader sequence;
b) an scFv that binds to FLT3;
c) a CD8a hinge domain;
d) a CD8a transmembrane domain;
e) a 4-1BB intracellular signaling sequence; and
f) a CD3ζ intracellular T cell signaling sequence.

Embodiment 5. The composition of embodiment 4, wherein the first polypeptide comprises the amino acid sequence put forth in SEQ ID NO: 139 and the second polypeptide comprises the amino acid sequence put forth in SEQ ID NO: 140.

Embodiment 6. A composition comprising a first polypeptide and a second polypeptide,

wherein the first polypeptide comprises:
a) an scFv that binds to CD19;
b) a CD28 hinge region;
c) a CD28 transmembrane domain;
d) a CD28 intracellular signaling sequence;
e) a CD3ζ intracellular T cell signaling sequence; and

wherein the second polypeptide comprises
a) an scFv that binds to FLT3;
b) a CD8a hinge domain;
c) a CD8a transmembrane domain;
d) a 4-1BB intracellular signaling sequence; and
e) a CD3ζ intracellular T cell signaling sequence.

Embodiment 7. The composition of embodiment 6, wherein the first polypeptide comprises the amino acid sequence put forth in SEQ ID NO: 141 and the second polypeptide comprises the amino acid sequence put forth in SEQ ID NO: 142.

Embodiment 8. A nucleic acid molecule comprising:

a) a nucleic acid sequence encoding a first leader sequence;

b) a nucleic acid sequence encoding an scFv that binds to CD33;

c) a nucleic acid sequence encoding a CD28 hinge region;

d) a nucleic acid encoding a CD28 transmembrane domain;

e) a nucleic acid sequence encoding a CD28 intracellular signaling sequence;

f) a nucleic acid sequence encoding a CD3ζ intracellular T cell signaling sequence;

g) a nucleic acid sequence encoding a self-cleaving linker peptide;

h) a nucleic acid sequence encoding a second leader sequence;

i) a nucleic acid sequence encoding an scFv that binds to FLT3;

j) a nucleic acid sequence encoding a CD8a hinge domain;

k) a nucleic acid sequence encoding a CD8a transmembrane domain;

l) a nucleic acid sequence encoding a 4-1BB intracellular signaling sequence; and

m) a nucleic acid sequence encoding a CD3ζ intracellular T cell signaling sequence.

Embodiment 9. A polypeptide comprising:

a) a first leader sequence;

b) an scFv that binds to CD33;

c) a CD28 hinge region;

d) a CD28 transmembrane domain;

e) a CD28 intracellular signaling sequence;

f) a CD3ζ intracellular T cell signaling sequence;

g) a self-cleaving linker peptide;

h) a second leader sequence;

i) an scFv that binds to FLT3;

j) a CD8a hinge domain;

k) a CD8a transmembrane domain;

l) a 4-1BB intracellular signaling sequence; and

m) a CD3ζ intracellular T cell signaling sequence.

Embodiment 10. The polypeptide of embodiment 9, wherein the polypeptide comprises the amino acid sequence put forth in SEQ ID NO: 143.

Embodiment 11. A composition comprising a first polypeptide and a second polypeptide,

wherein the first polypeptide comprises:
a) a first leader sequence;
b) an scFv that binds to CD33;
c) a CD28 hinge region;
d) a CD28 transmembrane domain;
e) a CD28 intracellular signaling sequence;
f) a CD3ζ intracellular T cell signaling sequence; and

wherein the second polypeptide comprises:
a) a second leader sequence;
b) an scFv that binds to FLT3;
c) a CD8a hinge domain;
d) a CD8a transmembrane domain;
e) a 4-1BB intracellular signaling sequence; and
f) a CD3ζ intracellular T cell signaling sequence.

Embodiment 12. The composition of embodiment 11, wherein the first polypeptide comprises the amino acid sequence put forth in SEQ ID NO: 144 and the second polypeptide comprises the amino acid sequence put forth in SEQ ID NO: 145.

Embodiment 13. A composition comprising a first polypeptide and a second polypeptide,

wherein the first polypeptide comprises:
a) an scFv that binds to CD33;
b) a CD28 hinge region;
c) a CD28 transmembrane domain;
d) a CD28 intracellular signaling sequence;
e) a CD3ζ intracellular T cell signaling sequence; and

wherein the second polypeptide comprises
a) an scFv that binds to FLT3;
b) a CD8a hinge domain;
c) a CD8a transmembrane domain;
d) a 4-1BB intracellular signaling sequence; and
e) a CD3ζ intracellular T cell signaling sequence.

Embodiment 14. The composition of embodiment 13, wherein the first polypeptide comprises the amino acid sequence put forth in SEQ ID NO: 146 and the second polypeptide comprises the amino acid sequence put forth in SEQ ID NO: 147.

Embodiment 15. A nucleic acid molecule comprising:

a) a nucleic acid sequence encoding a first leader sequence;

b) a nucleic acid sequence encoding an scFv that binds to CD19;

c) a nucleic acid sequence encoding a CD8a hinge domain;

d) a nucleic acid sequence encoding a CD8a transmembrane domain;

e) a nucleic acid sequence encoding a 4-1BB intracellular signaling sequence;

f) a nucleic acid sequence encoding a CD3ζ intracellular T cell signaling sequence;

g) a nucleic acid sequence encoding a self-cleaving linker peptide;

h) a nucleic acid sequence encoding a second leader sequence;

i) a nucleic acid sequence encoding an scFv that binds to FLT3;

j) a nucleic acid sequence encoding a CD8a hinge domain;

k) a nucleic acid sequence encoding a CD8a transmembrane domain;

l) a nucleic acid sequence encoding a 4-1BB intracellular signaling sequence; and

m) a nucleic acid sequence encoding a CD3ζ intracellular T cell signaling sequence.

Embodiment 16. A polypeptide comprising:

a) a first leader sequence;

b) an scFv that binds to CD19;

c) a CD8a hinge domain;

d) a CD8a transmembrane domain;

e) a 4-1BB intracellular signaling sequence;

f) a CD3ζ intracellular T cell signaling sequence;

g) a self-cleaving linker peptide;

h) a second leader sequence;

i) an scFv that binds to FLT3;

j) a CD8a hinge domain;

k) a CD8a transmembrane domain;

l) a 4-1BB intracellular signaling sequence; and

m) a CD3ζ intracellular T cell signaling sequence.

Embodiment 17. The polypeptide of embodiment 16, wherein the polypeptide comprises the amino acid sequence put forth in SEQ ID NO: 148.

Embodiment 18. A composition comprising a first polypeptide and a second polypeptide,

wherein the first polypeptide comprises:
a) a first leader sequence;
b) an scFv that binds to CD19;
c) a CD8a hinge domain;
d) a CD8a transmembrane domain;
e) a 4-1BB intracellular signaling sequence; and
f) a CD3ζ intracellular T cell signaling sequence; and

wherein the second polypeptide comprises:
a) a second leader sequence;
b) an scFv that binds to FLT3;
c) a CD8a hinge domain;
d) a CD8a transmembrane domain;
e) a 4-1BB intracellular signaling sequence; and
f) a CD3ζ intracellular T cell signaling sequence.

Embodiment 19. The composition of embodiment 18, wherein the first polypeptide comprises the amino acid sequence put forth in SEQ ID NO: 149 and the second polypeptide comprises the amino acid sequence put forth in SEQ ID NO: 150.

Embodiment 20. A composition comprising a first polypeptide and a second polypeptide,

wherein the first polypeptide comprises:
a) an scFv that binds to CD19;
b) a CD8a hinge domain;
c) a CD8a transmembrane domain;
d) a 4-1BB intracellular signaling sequence; and
e) a CD3ζ intracellular T cell signaling sequence; and

wherein the second polypeptide comprises
a) an scFv that binds to FLT3;
b) a CD8a hinge domain;
c) a CD8a transmembrane domain;
d) a 4-1BB intracellular signaling sequence; and
e) a CD3ζ intracellular T cell signaling sequence.

Embodiment 21. The composition of embodiment 20, wherein the first polypeptide comprises the amino acid sequence put forth in SEQ ID NO: 151 and the second polypeptide comprises the amino acid sequence put forth in SEQ ID NO: 152.

Embodiment 22. A nucleic acid molecule comprising:

a) a nucleic acid sequence encoding a first leader sequence;

b) a nucleic acid sequence encoding an scFv that binds to CD33;

c) a nucleic acid sequence encoding a CD8a hinge domain;

d) a nucleic acid sequence encoding a CD8a transmembrane domain;

e) a nucleic acid sequence encoding a 4-1BB intracellular signaling sequence;

f) a nucleic acid sequence encoding a CD3ζ intracellular T cell signaling sequence;

g) a nucleic acid sequence encoding a self-cleaving linker peptide;

h) a nucleic acid sequence encoding a second leader sequence;

i) a nucleic acid sequence encoding an scFv that binds to FLT3;

j) a nucleic acid sequence encoding a CD8a hinge domain;

k) a nucleic acid sequence encoding a CD8a transmembrane domain;

l) a nucleic acid sequence encoding a 4-1BB intracellular signaling sequence; and

m) a nucleic acid sequence encoding a CD3ζ intracellular T cell signaling sequence.

Embodiment 23. A polypeptide comprising:

a) a first leader sequence;

b) an scFv that binds to CD33;

c) a CD8a hinge domain;

d) a CD8a transmembrane domain;

e) a 4-1BB intracellular signaling sequence;

f) a CD3ζ intracellular T cell signaling sequence;

g) a self-cleaving linker peptide;

h) a second leader sequence;

i) an scFv that binds to FLT3;

j) a CD8a hinge domain;

k) a CD8a transmembrane domain;

l) a 4-1BB intracellular signaling sequence; and

m) a CD3ζ intracellular T cell signaling sequence.

Embodiment 24. The polypeptide of embodiment 23, wherein the polypeptide comprises the amino acid sequence put forth in SEQ ID NO: 153.

Embodiment 25. A composition comprising a first polypeptide and a second polypeptide,

wherein the first polypeptide comprises:
a) a first leader sequence;
b) an scFv that binds to CD33;
c) a CD8a hinge domain;
d) a CD8a transmembrane domain;
e) a 4-1BB intracellular signaling sequence; and
f) a CD3ζ intracellular T cell signaling sequence; and

wherein the second polypeptide comprises:
a) a second leader sequence;
b) an scFv that binds to FLT3;
c) a CD8a hinge domain;
d) a CD8a transmembrane domain;
e) a 4-1BB intracellular signaling sequence; and
f) a CD3ζ intracellular T cell signaling sequence.

Embodiment 26. The composition of embodiment 25, wherein the first polypeptide comprises the amino acid sequence put forth in SEQ ID NO: 154 and the second polypeptide comprises the amino acid sequence put forth in SEQ ID NO: 155.

Embodiment 27. A composition comprising a first polypeptide and a second polypeptide,

wherein the first polypeptide comprises:
a) an scFv that binds to CD33;
b) a CD8a hinge domain;
c) a CD8a transmembrane domain;
d) a 4-1BB intracellular signaling sequence; and
e) a CD3ζ intracellular T cell signaling sequence; and

wherein the second polypeptide comprises
a) an scFv that binds to FLT3;
b) a CD8a hinge domain;
c) a CD8a transmembrane domain;
d) a 4-1BB intracellular signaling sequence; and
e) a CD3ζ intracellular T cell signaling sequence.

Embodiment 28. The composition of embodiment 27, wherein the first polypeptide comprises the amino acid sequence put forth in SEQ ID NO: 156 and the second polypeptide comprises the amino acid sequence put forth in SEQ ID NO: 157.

Embodiment 29. The nucleic acid molecule, polypeptide or composition of any one of the preceding embodiments, wherein the first leader sequences comprises the amino acid sequence put forth in SEQ ID NO: 120.

Embodiment 30. The nucleic acid molecule, polypeptide or composition of any one of the preceding embodiments, wherein the first leader sequences comprises the amino acid sequence put forth in SEQ ID NO: 130.

Embodiment 31. The nucleic acid molecule, polypeptide or composition of any one of the preceding embodiments, wherein the scFv that binds to CD19 comprises the amino acid sequence put forth in SEQ ID NO: 33, the amino acid sequence put forth in SEQ ID NO: 67 and the amino acid sequence put forth in SEQ ID NO: 34.

Embodiment 32. The nucleic acid molecule, polypeptide or composition of any one of the preceding embodiments, wherein the scFv that binds to CD19 comprises the amino acid sequence put forth in SEQ ID NO: 121.

Embodiment 33. The nucleic acid molecule, polypeptide or composition of any one of the preceding embodiments, wherein the scFv that binds to CD33 comprises the amino acid sequence put forth in SEQ ID NO: 51, the amino acid sequence put forth in SEQ ID NO: 62 and the amino acid sequence put forth in SEQ ID NO: 52.

Embodiment 34. The nucleic acid molecule, polypeptide or composition of any one of the preceding embodiments, wherein the scFv that binds to CD33 comprises the amino acid sequence put forth in SEQ ID NO: 131.

Embodiment 35. The nucleic acid molecule, polypeptide or composition of any one of the preceding embodiments, wherein the CD28 hinge region comprises the amino acid sequence put forth in SEQ ID NO: 122.

Embodiment 36. The nucleic acid molecule, polypeptide or composition of any one of the preceding embodiments, wherein the CD28 transmembrane domain comprises the amino acid sequence put forth in SEQ ID NO: 79.

Embodiment 37. The nucleic acid molecule, polypeptide or composition of any one of the preceding embodiments, wherein the CD28 intracellular signaling sequence comprises the amino acid sequence put forth in SEQ ID NO: 123.

Embodiment 38. The nucleic acid molecule, polypeptide or composition of any one of the preceding embodiments, wherein the CD3ζ intracellular T cell signaling sequence comprises amino acid sequence put forth in SEQ ID NO: 74.

Embodiment 39. The nucleic acid molecule, polypeptide or composition of any one of the preceding embodiments, wherein the CD3ζ intracellular T cell signaling sequence comprises amino acid sequence put forth in SEQ ID NO: 124.

Embodiment 40. The nucleic acid molecule, polypeptide or composition of any one of the preceding embodiments, wherein the self-cleaving linker peptide comprises the amino acid sequence put forth in in SEQ ID NO: 58.

Embodiment 41. The nucleic acid molecule, polypeptide or composition of any one of the preceding embodiments, wherein the self-cleaving linker peptide comprises the amino acid sequence put forth in in SEQ ID NO: 125.

Embodiment 42. The nucleic acid molecule, polypeptide or composition of any one of the preceding embodiments, wherein the second leader sequence comprises the amino acid sequence put forth in SEQ ID NO: 126.

Embodiment 43. The nucleic acid molecule, polypeptide or composition of any one of the preceding embodiments, wherein the scFv that binds to FLT3 comprises the amino acid sequence put forth in SEQ ID NO: 18.

Embodiment 44. The nucleic acid molecule, polypeptide or composition of any one of the preceding embodiments, wherein the scFv that binds to FLT3 comprises the amino acid sequence put forth in SEQ ID NO: 127.

Embodiment 45. The nucleic acid molecule, polypeptide or composition of any one of the preceding embodiments, wherein the CD8a hinge domain comprises the amino acid sequence put forth in SEQ ID NO: 72.

Embodiment 46. The nucleic acid molecule, polypeptide or composition of any one of the preceding embodiments, wherein the CD8a transmembrane domain comprises the amino acid sequence put forth in SEQ ID NO: 71.

Embodiment 47. The nucleic acid molecule, polypeptide or composition of any one of the preceding embodiments, wherein the 4-1BB intracellular signaling sequence comprises the amino acid sequence put forth in SEQ ID NO: 73.

Embodiment 48. A nucleic acid molecule comprising:

a) a nucleic acid sequence encoding a first leader sequence, wherein the first leader sequences comprises the amino acid sequence put forth in SEQ ID NO: 120;

b) a nucleic acid sequence encoding an scFv that binds to CD19, wherein the scFv that binds to CD19 comprises the amino acid sequence put forth in SEQ ID NO: 121;

c) a nucleic acid sequence encoding a CD28 hinge region, wherein the CD28 hinge region comprises the amino acid sequence put forth in SEQ ID NO: 122;

d) a nucleic acid encoding a CD28 transmembrane domain, wherein the CD28 transmembrane domain comprises the amino acid sequence put forth in SEQ ID NO: 79;

e) a nucleic acid sequence encoding a CD28 intracellular signaling sequence, wherein the CD28 intracellular signaling sequence comprises the amino acid sequence put forth in SEQ ID NO: 123;

f) a nucleic acid sequence encoding a CD3ζ intracellular T cell signaling sequence, wherein the CD3ζ intracellular T cell signaling sequence comprises amino acid sequence put forth in SEQ ID NO: 124;

g) a nucleic acid sequence encoding a self-cleaving linker peptide, wherein the self-cleaving linker peptide comprises the amino acid sequence put forth in in SEQ ID NO: 125;

h) a nucleic acid sequence encoding a second leader sequence, wherein the second leader sequence comprises the amino acid sequence put forth in SEQ ID NO: 126;

i) a nucleic acid sequence encoding an scFv that binds to FLT3, wherein the scFv that binds to FLT3 comprises the amino acid sequence put forth in SEQ ID NO: 18;

j) a nucleic acid sequence encoding a CD8a hinge domain, wherein the CD8a hinge domain comprises the amino acid sequence put forth in SEQ ID NO: 72;

k) a nucleic acid sequence encoding a CD8a transmembrane domain, wherein the CD8a transmembrane domain comprises the amino acid sequence put forth in SEQ ID NO: 71;

l) a nucleic acid sequence encoding a 4-1BB intracellular signaling sequence, wherein the 4-1BB intracellular signaling sequence comprises the amino acid sequence put forth in SEQ ID NO: 73; and

m) a nucleic acid sequence encoding a CD3ζ intracellular T cell signaling sequence, wherein the CD3ζ intracellular T cell signaling sequence comprises amino acid sequence put forth in SEQ ID NO: 124.

Embodiment 49. A polypeptide comprising:

a) a first leader sequence, wherein the first leader sequences comprises the amino acid sequence put forth in SEQ ID NO: 120;

b) an scFv that binds to CD19, wherein the scFv that binds to CD19 comprises the amino acid sequence put forth in SEQ ID NO: 121;

c) a CD28 hinge region, wherein the CD28 hinge region comprises the amino acid sequence put forth in SEQ ID NO: 122;

d) a CD28 transmembrane domain, wherein the CD28 transmembrane domain comprises the amino acid sequence put forth in SEQ ID NO: 79;

e) a CD28 intracellular signaling sequence, wherein the CD28 intracellular signaling sequence comprises the amino acid sequence put forth in SEQ ID NO: 123;

f) a CD3ζ intracellular T cell signaling sequence, wherein the CD3ζ intracellular T cell signaling sequence comprises amino acid sequence put forth in SEQ ID NO: 124;

g) a self-cleaving linker peptide, wherein the self-cleaving linker peptide comprises the amino acid sequence put forth in in SEQ ID NO: 125;

h) a second leader sequence, wherein the second leader sequence comprises the amino acid sequence put forth in SEQ ID NO: 126;

i) an scFv that binds to FLT3, wherein the scFv that binds to FLT3 comprises the amino acid sequence put forth in SEQ ID NO: 18;

j) a CD8a hinge domain, wherein the CD8a hinge domain comprises the amino acid sequence put forth in SEQ ID NO: 72;

k) a CD8a transmembrane domain, wherein the CD8a transmembrane domain comprises the amino acid sequence put forth in SEQ ID NO: 71;

l) a 4-1BB intracellular signaling sequence, wherein the 4-1BB intracellular signaling sequence comprises the amino acid sequence put forth in SEQ ID NO: 73; and

m) a CD3ζ intracellular T cell signaling sequence, wherein the CD3ζ intracellular T cell signaling sequence comprises amino acid sequence put forth in SEQ ID NO: 124.

Embodiment 50. A composition comprising a first polypeptide and a second polypeptide,

wherein the first polypeptide comprises:

a) a first leader sequence, wherein the first leader sequences comprises the amino acid sequence put forth in SEQ ID NO: 120;

b) an scFv that binds to CD19, wherein the scFv that binds to CD19 comprises the amino acid sequence put forth in SEQ ID NO: 121;

c) a CD28 hinge region, wherein the CD28 hinge region comprises the amino acid sequence put forth in SEQ ID NO: 122;

d) a CD28 transmembrane domain, wherein the CD28 transmembrane domain comprises the amino acid sequence put forth in SEQ ID NO: 79;

e) a CD28 intracellular signaling sequence, wherein the CD28 intracellular signaling sequence comprises the amino acid sequence put forth in SEQ ID NO: 123;

f) a CD3ζ intracellular T cell signaling sequence, wherein the CD3ζ intracellular T cell signaling sequence comprises amino acid sequence put forth in SEQ ID NO: 124; and

wherein the second polypeptide comprises:

a) a second leader sequence, wherein the second leader sequence comprises the amino acid sequence put forth in SEQ ID NO: 126;

b) an scFv that binds to FLT3, wherein the scFv that binds to FLT3 comprises the amino acid sequence put forth in SEQ ID NO: 18;

c) a CD8a hinge domain, wherein the CD8a hinge domain comprises the amino acid sequence put forth in SEQ ID NO: 72;

d) a CD8a transmembrane domain, wherein the CD8a transmembrane domain comprises the amino acid sequence put forth in SEQ ID NO: 71;

e) a 4-1BB intracellular signaling sequence, wherein the 4-1BB intracellular signaling sequence comprises the amino acid sequence put forth in SEQ ID NO: 73; and

f) a CD3ζ intracellular T cell signaling sequence, wherein the CD3ζ intracellular T cell signaling sequence comprises amino acid sequence put forth in SEQ ID NO: 124.

Embodiment 51. A composition comprising a first polypeptide and a second polypeptide,

wherein the first polypeptide comprises:

a) an scFv that binds to CD19, wherein the scFv that binds to CD19 comprises the amino acid sequence put forth in SEQ ID NO: 121;

b) a CD28 hinge region, wherein the CD28 hinge region comprises the amino acid sequence put forth in SEQ ID NO: 122;

c) a CD28 transmembrane domain, wherein the CD28 transmembrane domain comprises the amino acid sequence put forth in SEQ ID NO: 79;

d) a CD28 intracellular signaling sequence, wherein the CD28 intracellular signaling sequence comprises the amino acid sequence put forth in SEQ ID NO: 123;

e) a CD3ζ intracellular T cell signaling sequence, wherein the CD3ζ intracellular T cell signaling sequence comprises amino acid sequence put forth in SEQ ID NO: 124; and

wherein the second polypeptide comprises:

a) an scFv that binds to FLT3, wherein the scFv that binds to FLT3 comprises the amino acid sequence put forth in SEQ ID NO: 18;

b) a CD8a hinge domain, wherein the CD8a hinge domain comprises the amino acid sequence put forth in SEQ ID NO: 72;

c) a CD8a transmembrane domain, wherein the CD8a transmembrane domain comprises the amino acid sequence put forth in SEQ ID NO: 71;

d) a 4-1BB intracellular signaling sequence, wherein the 4-1BB intracellular signaling sequence comprises the amino acid sequence put forth in SEQ ID NO: 73; and

e) a CD3ζ intracellular T cell signaling sequence, wherein the CD3ζ intracellular T cell signaling sequence comprises amino acid sequence put forth in SEQ ID NO: 124.

Embodiment 52. A nucleic acid molecule comprising:

a) a nucleic acid sequence encoding a first leader sequence, wherein the first leader sequences comprises the amino acid sequence put forth in SEQ ID NO: 130;

b) a nucleic acid sequence encoding an scFv that binds to CD33, wherein the scFv that binds to CD33 comprises the amino acid sequence put forth in SEQ ID NO: 131;

c) a nucleic acid sequence encoding a CD28 hinge region, wherein the CD28 hinge region comprises the amino acid sequence put forth in SEQ ID NO: 122;

d) a nucleic acid encoding a CD28 transmembrane domain, wherein the CD28 transmembrane domain comprises the amino acid sequence put forth in SEQ ID NO: 79;

e) a nucleic acid sequence encoding a CD28 intracellular signaling sequence, wherein the CD28 intracellular signaling sequence comprises the amino acid sequence put forth in SEQ ID NO: 123;

f) a nucleic acid sequence encoding a CD3ζ intracellular T cell signaling sequence, wherein the CD3ζ intracellular T cell signaling sequence comprises amino acid sequence put forth in SEQ ID NO: 124;

g) a nucleic acid sequence encoding a self-cleaving linker peptide, wherein the self-cleaving linker peptide comprises the amino acid sequence put forth in in SEQ ID NO: 125;

h) a nucleic acid sequence encoding a second leader sequence, wherein the second leader sequence comprises the amino acid sequence put forth in SEQ ID NO: 126;

i) a nucleic acid sequence encoding an scFv that binds to FLT3, wherein the scFv that binds to FLT3 comprises the amino acid sequence put forth in SEQ ID NO: 18;

j) a nucleic acid sequence encoding a CD8a hinge domain, wherein the CD8a hinge domain comprises the amino acid sequence put forth in SEQ ID NO: 72;

k) a nucleic acid sequence encoding a CD8a transmembrane domain, wherein the CD8a transmembrane domain comprises the amino acid sequence put forth in SEQ ID NO: 71;

l) a nucleic acid sequence encoding a 4-1BB intracellular signaling sequence, wherein the 4-1BB intracellular signaling sequence comprises the amino acid sequence put forth in SEQ ID NO: 73; and

m) a nucleic acid sequence encoding a CD3ζ intracellular T cell signaling sequence, wherein the CD3ζ intracellular T cell signaling sequence comprises amino acid sequence put forth in SEQ ID NO: 124.

Embodiment 53. A polypeptide comprising:

a) a first leader sequence, wherein the first leader sequences comprises the amino acid sequence put forth in SEQ ID NO: 130;

b) an scFv that binds to CD33, wherein the scFv that binds to CD33 comprises the amino acid sequence put forth in SEQ ID NO: 131;

c) a CD28 hinge region, wherein the CD28 hinge region comprises the amino acid sequence put forth in SEQ ID NO: 122;

d) a CD28 transmembrane domain, wherein the CD28 transmembrane domain comprises the amino acid sequence put forth in SEQ ID NO: 79;

e) a CD28 intracellular signaling sequence, wherein the CD28 intracellular signaling sequence comprises the amino acid sequence put forth in SEQ ID NO: 123;

f) a CD3ζ intracellular T cell signaling sequence, wherein the CD3ζ intracellular T cell signaling sequence comprises amino acid sequence put forth in SEQ ID NO: 124;

g) a self-cleaving linker peptide, wherein the self-cleaving linker peptide comprises the amino acid sequence put forth in in SEQ ID NO: 125;

h) a second leader sequence, wherein the second leader sequence comprises the amino acid sequence put forth in SEQ ID NO: 126;

i) an scFv that binds to FLT3, wherein the scFv that binds to FLT3 comprises the amino acid sequence put forth in SEQ ID NO: 18;

j) a CD8a hinge domain, wherein the CD8a hinge domain comprises the amino acid sequence put forth in SEQ ID NO: 72;

k) a CD8a transmembrane domain, wherein the CD8a transmembrane domain comprises the amino acid sequence put forth in SEQ ID NO: 71;

l) a 4-1BB intracellular signaling sequence, wherein the 4-1BB intracellular signaling sequence comprises the amino acid sequence put forth in SEQ ID NO: 73; and

m) a CD3ζ intracellular T cell signaling sequence, wherein the CD3ζ intracellular T cell signaling sequence comprises amino acid sequence put forth in SEQ ID NO: 124.

Embodiment 54. A composition comprising a first polypeptide and a second polypeptide,

wherein the first polypeptide comprises:

a) a first leader sequence, wherein the first leader sequences comprises the amino acid sequence put forth in SEQ ID NO: 130;

b) an scFv that binds to CD33, wherein the scFv that binds to CD33 comprises the amino acid sequence put forth in SEQ ID NO: 131;

c) a CD28 hinge region, wherein the CD28 hinge region comprises the amino acid sequence put forth in SEQ ID NO: 122;

d) a CD28 transmembrane domain, wherein the CD28 transmembrane domain comprises the amino acid sequence put forth in SEQ ID NO: 79;

e) a CD28 intracellular signaling sequence, wherein the CD28 intracellular signaling sequence comprises the amino acid sequence put forth in SEQ ID NO: 123;

f) a CD3ζ intracellular T cell signaling sequence, wherein the CD3ζ intracellular T cell signaling sequence comprises amino acid sequence put forth in SEQ ID NO: 124; and

wherein the second polypeptide comprises:

a) a second leader sequence, wherein the second leader sequence comprises the amino acid sequence put forth in SEQ ID NO: 126;

b) an scFv that binds to FLT3, wherein the scFv that binds to FLT3 comprises the amino acid sequence put forth in SEQ ID NO: 18;

c) a CD8a hinge domain, wherein the CD8a hinge domain comprises the amino acid sequence put forth in SEQ ID NO: 72;

d) a CD8a transmembrane domain, wherein the CD8a transmembrane domain comprises the amino acid sequence put forth in SEQ ID NO: 71;

e) a 4-1BB intracellular signaling sequence, wherein the 4-1BB intracellular signaling sequence comprises the amino acid sequence put forth in SEQ ID NO: 73; and

f) a CD3ζ intracellular T cell signaling sequence, wherein the CD3ζ intracellular T cell signaling sequence comprises amino acid sequence put forth in SEQ ID NO: 124.

Embodiment 55. A composition comprising a first polypeptide and a second polypeptide,

wherein the first polypeptide comprises:

a) an scFv that binds to CD33, wherein the scFv that binds to CD33 comprises the amino acid sequence put forth in SEQ ID NO: 131;

b) a CD28 hinge region, wherein the CD28 hinge region comprises the amino acid sequence put forth in SEQ ID NO: 122;

c) a CD28 transmembrane domain, wherein the CD28 transmembrane domain comprises the amino acid sequence put forth in SEQ ID NO: 79;

d) a CD28 intracellular signaling sequence, wherein the CD28 intracellular signaling sequence comprises the amino acid sequence put forth in SEQ ID NO: 123;

e) a CD3ζ intracellular T cell signaling sequence, wherein the CD3ζ intracellular T cell signaling sequence comprises amino acid sequence put forth in SEQ ID NO: 124; and

wherein the second polypeptide comprises:

a) an scFv that binds to FLT3, wherein the scFv that binds to FLT3 comprises the amino acid sequence put forth in SEQ ID NO: 18;

b) a CD8a hinge domain, wherein the CD8a hinge domain comprises the amino acid sequence put forth in SEQ ID NO: 72;

c) a CD8a transmembrane domain, wherein the CD8a transmembrane domain comprises the amino acid sequence put forth in SEQ ID NO: 71;

d) a 4-1BB intracellular signaling sequence, wherein the 4-1BB intracellular signaling sequence comprises the amino acid sequence put forth in SEQ ID NO: 73; and

e) a CD3ζ intracellular T cell signaling sequence, wherein the CD3ζ intracellular T cell signaling sequence comprises amino acid sequence put forth in SEQ ID NO: 124.

Embodiment 56. A nucleic acid molecule comprising:

a) a nucleic acid sequence encoding a first leader sequence, wherein the first leader sequence comprises the amino acid sequence put forth in SEQ ID NO: 120;

b) a nucleic acid sequence encoding an scFv that binds to CD19, wherein the scFv that binds to CD19 comprises the amino acid sequence put forth in SEQ ID NO: 121;

c) a nucleic acid sequence encoding a CD8a hinge domain, wherein the CD8a hinge domain comprises the amino acid sequence put forth in SEQ ID NO: 72;

d) a nucleic acid sequence encoding a CD8a transmembrane domain, wherein the CD8a transmembrane domain comprises the amino acid sequence put forth in SEQ ID NO: 71;

e) a nucleic acid sequence encoding a 4-1BB intracellular signaling sequence, wherein the 4-1BB intracellular signaling sequence comprises the amino acid sequence put forth in SEQ ID NO: 73;

f) a nucleic acid sequence encoding a CD3ζ intracellular T cell signaling sequence, wherein the CD3ζ intracellular T cell signaling sequence comprises amino acid sequence put forth in SEQ ID NO: 124;

g) a nucleic acid sequence encoding a self-cleaving linker peptide, wherein the self-cleaving linker peptide comprises the amino acid sequence put forth in in SEQ ID NO: 125;

h) a nucleic acid sequence encoding a second leader sequence, wherein the second leader sequence comprises the amino acid sequence put forth in SEQ ID NO: 126;

i) a nucleic acid sequence encoding an scFv that binds to FLT3 wherein the scFv that binds to FLT3 comprises the amino acid sequence put forth in SEQ ID NO: 18;

j) a nucleic acid sequence encoding a CD8a hinge domain, wherein the CD8a hinge domain comprises the amino acid sequence put forth in SEQ ID NO: 72;

k) a nucleic acid sequence encoding a CD8a transmembrane domain, wherein the CD8a transmembrane domain comprises the amino acid sequence put forth in SEQ ID NO: 71;

l) a nucleic acid sequence encoding a 4-1BB intracellular signaling sequence, wherein the 4-1BB intracellular signaling sequence comprises the amino acid sequence put forth in SEQ ID NO: 73; and

m) a nucleic acid sequence encoding a CD3ζ intracellular T cell signaling sequence, wherein the CD3ζ intracellular T cell signaling sequence comprises amino acid sequence put forth in SEQ ID NO: 124.

Embodiment 57. A polypeptide comprising:

a) a first leader sequence, wherein the first leader sequence comprises the amino acid sequence put forth in SEQ ID NO: 120;

b) an scFv that binds to CD19, wherein the scFv that binds to CD19 comprises the amino acid sequence put forth in SEQ ID NO: 121;

c) a CD8a hinge domain, wherein the CD8a hinge domain comprises the amino acid sequence put forth in SEQ ID NO: 72;

d) a CD8a transmembrane domain, wherein the CD8a transmembrane domain comprises the amino acid sequence put forth in SEQ ID NO: 71;

e) a 4-1BB intracellular signaling sequence, wherein the 4-1BB intracellular signaling sequence comprises the amino acid sequence put forth in SEQ ID NO: 73;

f) a CD3ζ intracellular T cell signaling sequence, wherein the CD3ζ intracellular T cell signaling sequence comprises amino acid sequence put forth in SEQ ID NO: 124;

g) a self-cleaving linker peptide, wherein the self-cleaving linker peptide comprises the amino acid sequence put forth in in SEQ ID NO: 125;

h) a second leader sequence, wherein the second leader sequence comprises the amino acid sequence put forth in SEQ ID NO: 126;

i) an scFv that binds to FLT3 wherein the scFv that binds to FLT3 comprises the amino acid sequence put forth in SEQ ID NO: 18;

j) a CD8a hinge domain, wherein the CD8a hinge domain comprises the amino acid sequence put forth in SEQ ID NO: 72;

k) a CD8a transmembrane domain, wherein the CD8a transmembrane domain comprises the amino acid sequence put forth in SEQ ID NO: 71;

l) a 4-1BB intracellular signaling sequence, wherein the 4-1BB intracellular signaling sequence comprises the amino acid sequence put forth in SEQ ID NO: 73; and

m) a CD3ζ intracellular T cell signaling sequence, wherein the CD3ζ intracellular T cell signaling sequence comprises amino acid sequence put forth in SEQ ID NO: 124.

Embodiment 58. A composition comprising a first polypeptide and a second polypeptide,

wherein the first polypeptide comprises:

a) a first leader sequence, wherein the first leader sequence comprises the amino acid sequence put forth in SEQ ID NO: 120;

b) an scFv that binds to CD19, wherein the scFv that binds to CD19 comprises the amino acid sequence put forth in SEQ ID NO: 121;

c) a CD8a hinge domain, wherein the CD8a hinge domain comprises the amino acid sequence put forth in SEQ ID NO: 72;

d) a CD8a transmembrane domain, wherein the CD8a transmembrane domain comprises the amino acid sequence put forth in SEQ ID NO: 71;

e) a 4-1BB intracellular signaling sequence, wherein the 4-1BB intracellular signaling sequence comprises the amino acid sequence put forth in SEQ ID NO: 73;

f) a CD3ζ intracellular T cell signaling sequence, wherein the CD3ζ intracellular T cell signaling sequence comprises amino acid sequence put forth in SEQ ID NO: 124; and

wherein the second polypeptide comprises:

a) a second leader sequence, wherein the second leader sequence comprises the amino acid sequence put forth in SEQ ID NO: 126;

b) an scFv that binds to FLT3 wherein the scFv that binds to FLT3 comprises the amino acid sequence put forth in SEQ ID NO: 18;

c) a CD8a hinge domain, wherein the CD8a hinge domain comprises the amino acid sequence put forth in SEQ ID NO: 72;

d) a CD8a transmembrane domain, wherein the CD8a transmembrane domain comprises the amino acid sequence put forth in SEQ ID NO: 71;

e) a 4-1BB intracellular signaling sequence, wherein the 4-1BB intracellular signaling sequence comprises the amino acid sequence put forth in SEQ ID NO: 73; and

f) a CD3ζ intracellular T cell signaling sequence, wherein the CD3ζ intracellular T cell signaling sequence comprises amino acid sequence put forth in SEQ ID NO: 124.

Embodiment 59. A composition comprising a first polypeptide and a second polypeptide,

wherein the first polypeptide comprises:

a) an scFv that binds to CD19, wherein the scFv that binds to CD19 comprises the amino acid sequence put forth in SEQ ID NO: 121;

b) a CD8a hinge domain, wherein the CD8a hinge domain comprises the amino acid sequence put forth in SEQ ID NO: 72;

c) a CD8a transmembrane domain, wherein the CD8a transmembrane domain comprises the amino acid sequence put forth in SEQ ID NO: 71;

d) a 4-1BB intracellular signaling sequence, wherein the 4-1BB intracellular signaling sequence comprises the amino acid sequence put forth in SEQ ID NO: 73;

e) a CD3ζ intracellular T cell signaling sequence, wherein the CD3ζ intracellular T cell signaling sequence comprises amino acid sequence put forth in SEQ ID NO: 124;

and

wherein the second polypeptide comprises:

a) an scFv that binds to FLT3 wherein the scFv that binds to FLT3 comprises the amino acid sequence put forth in SEQ ID NO: 18;

b) a CD8a hinge domain, wherein the CD8a hinge domain comprises the amino acid sequence put forth in SEQ ID NO: 72;

c) a CD8a transmembrane domain, wherein the CD8a transmembrane domain comprises the amino acid sequence put forth in SEQ ID NO: 71;

d) a 4-1BB intracellular signaling sequence, wherein the 4-1BB intracellular signaling sequence comprises the amino acid sequence put forth in SEQ ID NO: 73; and

e) a CD3ζ intracellular T cell signaling sequence, wherein the CD3ζ intracellular T cell signaling sequence comprises amino acid sequence put forth in SEQ ID NO: 124.

Embodiment 60. A nucleic acid molecule comprising:

a) a nucleic acid sequence encoding a first leader sequence, wherein the first leader sequence comprises the amino acid sequence put forth in SEQ ID NO: 130;

b) a nucleic acid sequence encoding an scFv that binds to CD33, wherein the scFv that binds to CD33 comprises the amino acid sequence put forth in SEQ ID NO: 131;

c) a nucleic acid sequence encoding a CD8a hinge domain, wherein the CD8a hinge domain comprises the amino acid sequence put forth in SEQ ID NO: 72;

d) a nucleic acid sequence encoding a CD8a transmembrane domain, wherein the CD8a transmembrane domain comprises the amino acid sequence put forth in SEQ ID NO: 71;

e) a nucleic acid sequence encoding a 4-1BB intracellular signaling sequence, wherein the 4-1BB intracellular signaling sequence comprises the amino acid sequence put forth in SEQ ID NO: 73;

f) a nucleic acid sequence encoding a CD3ζ intracellular T cell signaling sequence, wherein the CD3ζ intracellular T cell signaling sequence comprises amino acid sequence put forth in SEQ ID NO: 124;

g) a nucleic acid sequence encoding a self-cleaving linker peptide, wherein the self-cleaving linker peptide comprises the amino acid sequence put forth in in SEQ ID NO: 125;

h) a nucleic acid sequence encoding a second leader sequence, wherein the second leader sequence comprises the amino acid sequence put forth in SEQ ID NO: 126;

i) a nucleic acid sequence encoding an scFv that binds to FLT3 wherein the scFv that binds to FLT3 comprises the amino acid sequence put forth in SEQ ID NO: 18;

j) a nucleic acid sequence encoding a CD8a hinge domain, wherein the CD8a hinge domain comprises the amino acid sequence put forth in SEQ ID NO: 72;

k) a nucleic acid sequence encoding a CD8a transmembrane domain, wherein the CD8a transmembrane domain comprises the amino acid sequence put forth in SEQ ID NO: 71;

l) a nucleic acid sequence encoding a 4-1BB intracellular signaling sequence, wherein the 4-1BB intracellular signaling sequence comprises the amino acid sequence put forth in SEQ ID NO: 73; and

m) a nucleic acid sequence encoding a CD3ζ intracellular T cell signaling sequence, wherein the CD3ζ intracellular T cell signaling sequence comprises amino acid sequence put forth in SEQ ID NO: 124.

Embodiment 61. A polypeptide comprising:

a) a first leader sequence, wherein the first leader sequence comprises the amino acid sequence put forth in SEQ ID NO: 130;

b) an scFv that binds to CD33, wherein the scFv that binds to CD33 comprises the amino acid sequence put forth in SEQ ID NO: 131;

c) a CD8a hinge domain, wherein the CD8a hinge domain comprises the amino acid sequence put forth in SEQ ID NO: 72;

d) a CD8a transmembrane domain, wherein the CD8a transmembrane domain comprises the amino acid sequence put forth in SEQ ID NO: 71;

e) a 4-1BB intracellular signaling sequence, wherein the 4-1BB intracellular signaling sequence comprises the amino acid sequence put forth in SEQ ID NO: 73;

f) a CD3ζ intracellular T cell signaling sequence, wherein the CD3ζ intracellular T cell signaling sequence comprises amino acid sequence put forth in SEQ ID NO: 124;

g) a self-cleaving linker peptide, wherein the self-cleaving linker peptide comprises the amino acid sequence put forth in in SEQ ID NO: 125;

h) a second leader sequence, wherein the second leader sequence comprises the amino acid sequence put forth in SEQ ID NO: 126;

i) an scFv that binds to FLT3 wherein the scFv that binds to FLT3 comprises the amino acid sequence put forth in SEQ ID NO: 18;

j) a CD8a hinge domain, wherein the CD8a hinge domain comprises the amino acid sequence put forth in SEQ ID NO: 72;

k) a CD8a transmembrane domain, wherein the CD8a transmembrane domain comprises the amino acid sequence put forth in SEQ ID NO: 71;

l) a 4-1BB intracellular signaling sequence, wherein the 4-1BB intracellular signaling sequence comprises the amino acid sequence put forth in SEQ ID NO: 73; and

m) a CD3ζ intracellular T cell signaling sequence, wherein the CD3ζ intracellular T cell signaling sequence comprises amino acid sequence put forth in SEQ ID NO: 124.

Embodiment 62. A composition comprising a first polypeptide and a second polypeptide,

wherein the first polypeptide comprises:

a) a first leader sequence, wherein the first leader sequence comprises the amino acid sequence put forth in SEQ ID NO: 130;

b) an scFv that binds to CD33, wherein the scFv that binds to CD33 comprises the amino acid sequence put forth in SEQ ID NO: 131;

c) a CD8a hinge domain, wherein the CD8a hinge domain comprises the amino acid sequence put forth in SEQ ID NO: 72;

d) a CD8a transmembrane domain, wherein the CD8a transmembrane domain comprises the amino acid sequence put forth in SEQ ID NO: 71;

e) a 4-1BB intracellular signaling sequence, wherein the 4-1BB intracellular signaling sequence comprises the amino acid sequence put forth in SEQ ID NO: 73;

f) a CD3ζ intracellular T cell signaling sequence, wherein the CD3ζ intracellular T cell signaling sequence comprises amino acid sequence put forth in SEQ ID NO: 124;

and

wherein the second polypeptide comprises:

a) a second leader sequence, wherein the second leader sequence comprises the amino acid sequence put forth in SEQ ID NO: 126;

b) an scFv that binds to FLT3 wherein the scFv that binds to FLT3 comprises the amino acid sequence put forth in SEQ ID NO: 18;

c) a CD8a hinge domain, wherein the CD8a hinge domain comprises the amino acid sequence put forth in SEQ ID NO: 72;

d) a CD8a transmembrane domain, wherein the CD8a transmembrane domain comprises the amino acid sequence put forth in SEQ ID NO: 71;

e) a 4-1BB intracellular signaling sequence, wherein the 4-1BB intracellular signaling sequence comprises the amino acid sequence put forth in SEQ ID NO: 73; and

f) a CD3ζ intracellular T cell signaling sequence, wherein the CD3ζ intracellular T cell signaling sequence comprises amino acid sequence put forth in SEQ ID NO: 124.

Embodiment 63. A composition comprising a first polypeptide and a second polypeptide,

wherein the first polypeptide comprises:

a) an scFv that binds to CD33, wherein the scFv that binds to CD33 comprises the amino acid sequence put forth in SEQ ID NO: 131;

b) a CD8a hinge domain, wherein the CD8a hinge domain comprises the amino acid sequence put forth in SEQ ID NO: 72;

c) a CD8a transmembrane domain, wherein the CD8a transmembrane domain comprises the amino acid sequence put forth in SEQ ID NO: 71;

d) a 4-1BB intracellular signaling sequence, wherein the 4-1BB intracellular signaling sequence comprises the amino acid sequence put forth in SEQ ID NO: 73;

e) a CD3ζ intracellular T cell signaling sequence, wherein the CD3ζ intracellular T cell signaling sequence comprises amino acid sequence put forth in SEQ ID NO: 124;

and

wherein the second polypeptide comprises:

a) an scFv that binds to FLT3 wherein the scFv that binds to FLT3 comprises the amino acid sequence put forth in SEQ ID NO: 18;

b) a CD8a hinge domain, wherein the CD8a hinge domain comprises the amino acid sequence put forth in SEQ ID NO: 72;

c) a CD8a transmembrane domain, wherein the CD8a transmembrane domain comprises the amino acid sequence put forth in SEQ ID NO: 71;

d) a 4-1BB intracellular signaling sequence, wherein the 4-1BB intracellular signaling sequence comprises the amino acid sequence put forth in SEQ ID NO: 73; and

e) a CD3ζ intracellular T cell signaling sequence, wherein the CD3ζ intracellular T cell signaling sequence comprises amino acid sequence put forth in SEQ ID NO: 124.

Embodiment 64. The nucleic acid molecule of any of the preceding embodiments, wherein the nucleic acid molecule comprises the nucleic acid sequence put forth in SEQ ID NO: 128.

Embodiment 65. The nucleic acid molecule of any of the preceding embodiments, wherein the nucleic acid molecule comprises the nucleic acid sequence put forth in SEQ ID NO: 129.

Embodiment 66. The nucleic acid molecule of any of the preceding embodiments, wherein the nucleic acid molecule comprises the nucleic acid sequence put forth in SEQ ID NO: 132.

Embodiment 67. The nucleic acid molecule of any of the preceding embodiments, wherein the nucleic acid molecule comprises the nucleic acid sequence put forth in SEQ ID NO: 133.

Embodiment 68. The nucleic acid molecule of any of the preceding embodiments, wherein the nucleic acid molecule comprises the nucleic acid sequence put forth in SEQ ID NO: 134.

Embodiment 69. The nucleic acid molecule of any of the preceding embodiments, wherein the nucleic acid molecule comprises the nucleic acid sequence put forth in SEQ ID NO: 135.

Embodiment 70. The nucleic acid molecule of any of the preceding embodiments, wherein the nucleic acid molecule comprises the nucleic acid sequence put forth in SEQ ID NO: 136.

Embodiment 71. The nucleic acid molecule of any of the preceding embodiments, wherein the nucleic acid molecule comprises the nucleic acid sequence put forth in SEQ ID NO: 137.

Embodiment 72. A recombinant expression vector comprising the nucleic acid molecule of any one of the preceding embodiments.

Embodiment 73. A host cell comprising the recombinant expression vector, nucleic acid molecule, polypeptide or composition of any one of the preceding embodiments.

Embodiment 74. The host cell of any one of the preceding embodiments, wherein the host cell is a T-cell.

Embodiment 75. A population of cells comprising at least one of the host cells of any one of the preceding embodiments.

Embodiment 76. A method of treating acute lymphoblastic leukemia (ALL) or acute myeloid leukemia (AML) in a subject, the method comprising administering to the subject an effective amount of the nucleic acid molecule, recombinant expression vector, polypeptide, composition, host cell or population of cells of any one of the preceding embodiments.

Embodiment 77. A method of treating cancer in a subject, the method comprising administering to the subject an effective amount of the nucleic acid molecule, recombinant expression vector, polypeptide, composition, host cell or population of cells of any one of the preceding embodiments.

Embodiment 78. The nucleic acid molecule, recombinant expression vector, polypeptide, composition, host cell or population of cells of any one of the preceding embodiments for use in treating cancer in a subject.

Embodiment 79. The nucleic acid molecule, recombinant expression vector, polypeptide, composition, host cell or population of cells of any one of the preceding embodiments for use in treating acute lymphoblastic leukemia (ALL) or acute myeloid leukemia (AML) in a subject.

Embodiment 80. The method or use of any one of the preceding embodiments, wherein the ALL is B-cell ALL (B-ALL).

Embodiment 81. The method or use of any one of the preceding embodiments, wherein the effective amount of host cells is at least 1×10⁶ cells.

Embodiment 82. A bivalent chimeric antigen receptor (CAR) comprising:

a first anti-FLT3 antigen binding domain;

a second antigen binding domain selected from the group consisting of:

an anti-CD19 antigen binding domain, an anti-CD22 antigen binding domain, an anti-CD33 antigen binding domain and an anti-CD123 binding domain;

at least one linker domain;

a transmembrane domain; and

an intracellular T-cell signaling domain.

Embodiment 83. The bivalent CAR of any one of the preceding embodiments, wherein the first anti-FLT3 antigen binding domain comprises:

an anti-FLT3 single chain variable fragment (scFv) NC7 variable heavy chain fragment (SEQ ID NO: 16) and variable light chain fragment (SEQ ID NO: 17);

an anti-FLT3 single chain variable fragment (scFv) m1006 variable heavy chain fragment (SEQ ID NO: 82) and variable light chain fragment (SEQ ID NO: 83); or

an anti-FLT3 single chain variable fragment (scFv) m1007 variable heavy chain fragment (SEQ ID NO: 85) and variable light chain fragment (SEQ ID NO: 86).

Embodiment 84. The bivalent CAR of any one of the preceding embodiments, wherein the anti-CD19 antigen binding domain comprises an anti-CD19 scFv FMC63 variable heavy chain fragment (SEQ ID NO: 33) and variable light chain fragment (SEQ ID NO: 34).

Embodiment 85. The bivalent CAR of any one of the preceding embodiments, wherein the anti-CD22 antigen binding domain comprises anti-CD22 scFV m971 variable heavy chain fragment (SEQ ID NO: 49) and variable light chain fragment (SEQ ID NO: 50).

Embodiment 86. The bivalent CAR of any one of the preceding embodiments, wherein the anti-CD33 antigen binding domain comprises:

an anti-CD33 scFv Hum 195 variable heavy chain fragment (SEQ ID NO: 51) and variable light chain fragment (SEQ ID NO: 52); or

an anti-CD33 scFc h-p67.6 variable heavy chain fragment (SEQ ID NO: 55) and variable light chain fragment (SEQ ID NO: 56).

Embodiment 87. The bivalent CAR of any one of the preceding embodiments, wherein the anti-CD123 antigen binding domain comprises:

an anti-CD123 scFv 26292 variable heavy chain fragment (SEQ ID NO: 88) and variable light chain fragment (SEQ ID NO: 92);

an anti-CD123 scFv 32701 variable heavy chain fragment (SEQ ID NO: 96) and variable light chain fragment (SEQ ID NO: 100);

an anti-CD123 scFv 32716 variable heavy chain fragment (SEQ ID NO: 104) and variable light chain fragment (SEQ ID NO: 108); or

an anti-CD123 scFv 32703 variable heavy chain fragment (SEQ ID NO: 112) and variable light chain fragment (SEQ ID NO: 116).

Embodiment 88. The bivalent CAR of any one of the preceding embodiments, wherein the first anti-FLT3 antigen binding domain and the second antigen binding domain are arranged in tandem.

Embodiment 89. The bivalent CAR of any one of the preceding embodiments, wherein the first anti-FLT3 antigen binding domain is proximal to the transmembrane domain.

Embodiment 90. The bivalent CAR of any one of the preceding embodiments, wherein the second antigen binding domain is proximal to the transmembrane domain.

Embodiment 91. The bivalent CAR of any one of the preceding embodiments, wherein an internal linker in the first anti-FLT3 antigen binding domain forms a loop structure and the second antigen binding domain is proximal to the transmembrane domain.

Embodiment 92. The bivalent CAR of any one of the preceding embodiments, wherein an internal linker in the second antigen binding domain forms a loop structure and the first anti-FLT3 antigen binding domain is proximal to the transmembrane domain.

Embodiment 93. The bivalent CAR of any one of the preceding embodiments, wherein the transmembrane domain comprises a CD8a or CD28 transmembrane domain or a transmembrane fragment thereof.

Embodiment 94. The bivalent CAR of any one of the preceding embodiments, wherein the CD8a transmembrane domain comprises the amino acid sequence of SEQ ID NO: 71 and the CD28 transmembrane domain comprises the amino acid sequence of SEQ ID NO: 79.

Embodiment 95. The bivalent CAR of any one of the preceding embodiments, wherein the intracellular domain comprises a 4-1BB costimulatory domain comprising the amino acid sequence of SEQ ID NO: 73 or a CD28 costimulatory domain comprising the amino acid sequence of SEQ ID NO: 81, and a CD3ζ intracellular T-cell signaling sequence comprising the amino acid sequence of SEQ ID NO: 74.

Embodiment 96. The bivalent CAR of any one of the preceding embodiments, further comprising a nucleotide sequence encoding a hinge domain.

Embodiment 97. The bivalent CAR construct of any one of the preceding embodiments, wherein the hinge domain is a CD8 hinge domain comprising the amino acid sequence of SEQ ID NO: 72 or a CD28 hinge domain comprising the amino acid sequence of SEQ ID NO: 80.

Embodiment 98. An expression construct comprising a nucleotide sequence encoding a bivalent CAR according to any one of the preceding embodiments.

Embodiment 99. A recombinant expression vector comprising the expression construct of any one of the preceding embodiments.

Embodiment 100. A bivalent CAR expressed from the recombinant expression vector of any one of the preceding embodiments.

Embodiment 101. A host cell comprising the recombinant expression vector of any one of the preceding embodiments.

Embodiment 102. The host cell of any one of the preceding embodiments, wherein the host cell is a T-cell.

Embodiment 103. A population of cells comprising at least one host cell of any one of the preceding embodiments.

Embodiment 104. A method of treating acute lymphoblastic leukemia (ALL) or acute myeloid leukemia (AML) in a subject, the method comprising administering to the subject an effective amount of the host cells according to any one of the preceding embodiments.

Embodiment 105. The method of any one of the preceding embodiments, wherein the effective amount of host cells comprises at least 1×10⁶ cells.

Embodiment 106. The method of any one of the preceding embodiments, wherein the ALL is B-cell ALL (B-ALL).

Embodiment 107. A bivalent CAR of any one of the preceding embodiments for use in the treatment of acute lymphoblastic leukemia (ALL) or acute myeloid leukemia (AML).

Embodiment 108. The bivalent CAR of any one of the preceding embodiments, wherein the bivalent CAR is expressed on the surface of a T-cell.

Embodiment 109. The bivalent CAR of any one of the preceding embodiments, wherein treatment comprises administering to the subject an effective amount of the T-cells, optionally wherein the effective amount comprises at least 1×10⁶ cells.

Embodiment 110. A pair of chimeric antigen receptors (CAR) comprising:

a first CAR comprising:

an anti-FLT3 antigen binding domain;

a transmembrane domain; and

an intracellular T-cell signaling domain; and

a second CAR comprising:

an antigen binding domain selected from the group consisting of an anti-CD19 antigen binding domain; an anti-CD22 antigen binding domain; an anti-CD33 antigen binding domain and an anti-CD123 antigen binding domain;

a transmembrane domain; and

an intracellular T-cell signaling domain.

Embodiment 111. The pair of CARs of any one of the preceding embodiments, wherein the anti-FLT3 antigen binding domain of the first CAR comprises:

an anti-FLT3 single chain variable fragment (scFv) NC7 variable heavy chain fragment (SEQ ID NO: 16) and variable light chain fragment (SEQ ID NO: 17);

an anti-FLT3 single chain variable fragment (scFv) m1006 variable heavy chain fragment (SEQ ID NO: 82) and variable light chain fragment (SEQ ID NO: 83); or

an anti-FLT3 single chain variable fragment (scFv) m1007 variable heavy chain fragment (SEQ ID NO: 85) and variable light chain fragment (SEQ ID NO: 86).

Embodiment 112. The pair of CARs of any one of the preceding embodiments, wherein the second CAR comprises an anti-CD19 antigen binding domain comprising anti-CD19 scFv FMC63 variable heavy chain fragment (SEQ ID NO: 33) and variable light chain fragment (SEQ ID NO: 34).

Embodiment 113. The pair of CARs of any one of the preceding embodiments, wherein the second CAR comprises an anti-CD22 antigen binding domain comprising anti-CD22 scFV m971 variable heavy chain fragment (SEQ ID NO: 49) and variable light chain fragment (SEQ ID NO: 50).

Embodiment 114. The pair of CARs of any one of the preceding embodiments, wherein the second CAR comprises:

an anti-CD33 antigen binding domain comprising anti-CD33 HuM195 variable heavy chain fragment (SEQ ID NO: 51) and variable light chain fragment (SEQ ID NO: 52); or

an anti-CD33 scFc h-p67.6 variable heavy chain fragment (SEQ ID NO: 55) and variable light chain fragment (SEQ ID NO: 56).

Embodiment 115. The pair of CARs of any one of the preceding embodiments, wherein the second CAR comprises:

an anti-CD123 scFv 26292 variable heavy chain fragment (SEQ ID NO: 88) and variable light chain fragment (SEQ ID NO: 92);

an anti-CD123 scFv 32701 variable heavy chain fragment (SEQ ID NO: 96) and variable light chain fragment (SEQ ID NO: 100);

an anti-CD123 scFv 32716 variable heavy chain fragment (SEQ ID NO: 104) and variable light chain fragment (SEQ ID NO: 108); or

an anti-CD123 scFv 32703 variable heavy chain fragment (SEQ ID NO: 112) and variable light chain fragment (SEQ ID NO: 116).

Embodiment 116. The pair of CARs of any one of the preceding embodiments, wherein the first CAR and the second CAR each further comprise a CD8a or CD28 transmembrane domain or a transmembrane fragment thereof.

Embodiment 117. The pair of CARs of any one of the preceding embodiments, wherein the CD8a transmembrane domain comprises the amino acid sequence of SEQ ID NO: 71 and the CD28 transmembrane domain comprises the amino acid sequence of SEQ ID NO: 79.

Embodiment 118. The pair of CARs of any one of the preceding embodiments, wherein the intracellular T-cell signaling domain of each of the first CAR and the second CAR comprises a 4-1BB costimulatory domain comprising the amino acid sequence of SEQ ID NO: 73 or a CD28 costimulatory domain comprising the amino acid sequence of SEQ ID NO: 81, and a CD3ζ intracellular T-cell signaling sequence comprising the amino acid sequence of SEQ ID NO: 74.

Embodiment 119. The pair of CARS of any one of the preceding embodiments, wherein each of the first CAR and the second CAR further comprises a hinge domain.

Embodiment 120. The pair of CARS of any one of the preceding embodiments, wherein the hinge domain is a CD8 hinge domain comprising the amino acid sequence of SEQ ID NO: 72 or a CD28 hinge domain comprising the amino acid sequence of SEQ ID NO: 80.

Embodiment 121. An expression construct comprising a nucleotide sequence encoding the pair of CARs according to any one of the preceding embodiments, wherein a nucleotide sequence encoding the first CAR is joined to a nucleotide sequence encoding the second CAR by a nucleotide that encodes a self-cleaving linker peptide.

Embodiment 122. A recombinant expression vector comprising the expression construct of any one of the preceding embodiments.

Embodiment 123. A host cell comprising the pair of CARs of any one of the preceding embodiments and/or the recombinant expression vector of any one of the preceding embodiments

Embodiment 124. The host cell of any one of the preceding embodiments, wherein the host cell is a T-cell.

Embodiment 125. A population of cells comprising at least one host cell of any one of the preceding embodiments.

Embodiment 126. A method of treating acute lymphoblastic leukemia (ALL) or acute myeloid leukemia (AML) in a subject, the method comprising administering to the subject an effective amount of host cells according to any one of the preceding embodiments.

Embodiment 127. The method of any one of the preceding embodiments, wherein the effective amount of host cells is at least 1×10⁶ cells.

Embodiment 128. The method of any one of the preceding embodiments, wherein the ALL is B-cell ALL (B-ALL).

Embodiment 129. The pair of CARs of any one of the preceding embodiments for use in the treatment of acute lymphoblastic leukemia (ALL) or acute myeloid leukemia (AML).

Embodiment 130. The pair of CARs of any one of the preceding embodiments, wherein the CARs are expressed on the surface of a T-cell.

Embodiment 131. The pair of CARs of any one of the preceding embodiments, wherein treatment comprises administering an effective amount T-cells expressing the bivalent CAR of any one of the preceding embodiments, optionally wherein the effective amount comprises at least 1×10⁶ cells.

Embodiment 132. A bicistronic chimeric antigen receptor (CAR) construct comprising:

i) a first group of nucleotide sequences comprising:
a) a nucleotide sequence encoding a first anti-FLT3 antigen binding domain;
b) a nucleotide sequence encoding a first transmembrane domain; and
c) a nucleotide sequence encoding a first intracellular T-cell signaling domain;

ii) a second group of nucleotide sequences comprising:
a) a nucleotide sequence encoding an antigen binding domain selected from the group consisting of an anti-CD19 antigen binding domain, an anti-CD22 antigen binding domain, an anti-CD33 antigen binding domain and an anti-CD123 antigen binding domain;
b) a nucleotide sequence encoding a second transmembrane domain; and
c) a nucleotide sequence encoding a second intracellular T-cell signaling domain; and

iii) a cleavable linker nucleotide sequence between the first group of nucleotide sequences and the second group of nucleotide sequences encoding a cleavable linker peptide.

Embodiment 133. The bicistronic CAR construct of any one of the preceding embodiments, wherein the nucleotide sequence encoding the first anti-FLT3 antigen binding domain encodes

a variable heavy chain fragment (SEQ ID NO: 16) and a variable light chain fragment (SEQ ID NO: 17) of a single chain variable fragment (scFv) of anti-FLT3 NC7;

a variable heavy chain fragment (SEQ ID NO: 82) and a variable light chain fragment (SEQ ID NO: 83) of a scFv of anti-FLT3 m1006, or

a variable heavy chain fragment (SEQ ID NO: 85) and a variable light chain fragment (SEQ ID NO: 85) of a scFv of anti-FLT3 m1007.

Embodiment 134. The bicistronic CAR construct of any one of the preceding embodiments, wherein the nucleotide sequence encoding the second antigen binding domain encodes a variable heavy chain fragment (SEQ ID NO: 33) and variable light chain fragment (SEQ ID NO: 34) of a single chain variable fragment (scFv) of anti-CD19 FMC63.

Embodiment 135. The bicistronic CAR construct of any one of the preceding embodiments, wherein the nucleotide sequence encoding the second antigen binding domain encodes a variable heavy chain fragment (SEQ ID NO: 49) and variable light chain fragment (SEQ ID NO: 50) of a single chain variable fragment (scFv) of anti-CD22 m971.

Embodiment 136. The bicistronic CAR construct of any one of the preceding embodiments, wherein the nucleotide sequence encoding the second antigen binding domain encodes

a variable heavy chain fragment (SEQ ID NO: 51) and a variable light chain fragment (SEQ ID NO: 52) of a single chain variable fragment (scFv) of anti-CD33 HuM195, or

a variable heavy chain fragment (SEQ ID NO: 55) and a variable light chain fragment (SEQ ID NO: 56) of a single chain variable fragment (scFv) of anti-CD33 h-p67.6.

Embodiment 137. The bicistronic CAR construct of any one of the preceding embodiments, wherein the nucleotide sequence encoding the second antigen binding domain encodes:

a variable heavy chain fragment (SEQ ID NO: 88) and variable light chain fragment (SEQ ID NO: 92) of anti-CD123 scFv 26292;

a variable heavy chain fragment (SEQ ID NO: 96) and variable light chain fragment (SEQ ID NO: 100) of anti-CD123 scFv 32701;

a heavy chain fragment (SEQ ID NO: 104) and variable light chain fragment (SEQ ID NO: 108) of anti-CD123 scFv 32716; or

a heavy chain fragment (SEQ ID NO: 112) and variable light chain fragment (SEQ ID NO: 116) of anti-CD123 scFv 32703.

Embodiment 138. The bicistronic CAR construct of any one of the preceding embodiments, wherein:

both of the first and second transmembrane domains comprise a CD8 transmembrane domain comprising the amino acid sequence of SEQ ID NO: 71 or a CD28 transmembrane domain comprising the amino acid sequence of SEQ ID NO: 79, both of the first and second intracellular T-cell signaling domains comprise a 4-1BB costimulatory domain comprising the amino acid sequence of SEQ ID NO: 73 or a CD28 costimulatory domain comprising the amino acid sequence of SEQ ID NO: 81, and a CD3ζ intracellular T-cell signaling sequence comprising the amino acid sequence of SEQ ID NO: 74, and wherein the first group of nucleotide sequences and the second group of nucleotide sequences each further comprise a nucleotide sequence encoding a hinge domain, wherein the hinge domain is a CD8 hinge domain comprising the amino acid sequence of SEQ ID NO: 72 or a CD28 hinge domain comprising the amino acid sequence of SEQ ID NO: 80.

Embodiment 139. The bicistronic CAR construct of any one of the preceding embodiments, wherein the self-cleaving linker peptide is a 2A linker selected from the group consisting of T2A (EGRGSLLTCGDVEENPGP; SEQ ID NO: 75), P2A (ATNFSLLKQAGDVEENPGP; SEQ ID NO: 76), E2A (QCTNYALLKLAGDVESNPGP; SEQ ID NO: 77) and F2A (VKQTLNFDLLKLAGDVESNPGP; SEQ ID NO: 78), or a furin-cleavable linker.

Embodiment 140. A recombinant expression vector comprising the bicistronic CAR construct of any one of the preceding embodiments.

Embodiment 141. A host cell comprising the recombinant expression vector of any one of the preceding embodiments.

Embodiment 142. The host cell of any one of the preceding embodiments, wherein the host cell is a T-cell.

Embodiment 143. A population of cells comprising at least one host cell of any one of the preceding embodiments.

Embodiment 144. A method of treating acute lymphoblastic leukemia (ALL) or acute myeloid leukemia (AML) in a subject, the method comprising administering to the subject an effective amount of host cells according to any one of the preceding embodiments.

Embodiment 145. The method of any one of the preceding embodiments, wherein the effective amount of host cells is at least 1×10⁶ cells.

Embodiment 146. The method of any one of the preceding embodiments, wherein the ALL is B-cell ALL (B-ALL).

Embodiment 147. The host cell of any one of the preceding embodiments for use in the treatment of acute lymphoblastic leukemia (ALL) or acute myeloid leukemia (AML).

### Example 1—Bicistronic CAR Constructs of the Present Disclosure are Expressed in T-Cells

The following is a non-limiting example that demonstrates that the bicistronic CAR constructs of the present disclosure can be expressed in T-cells.

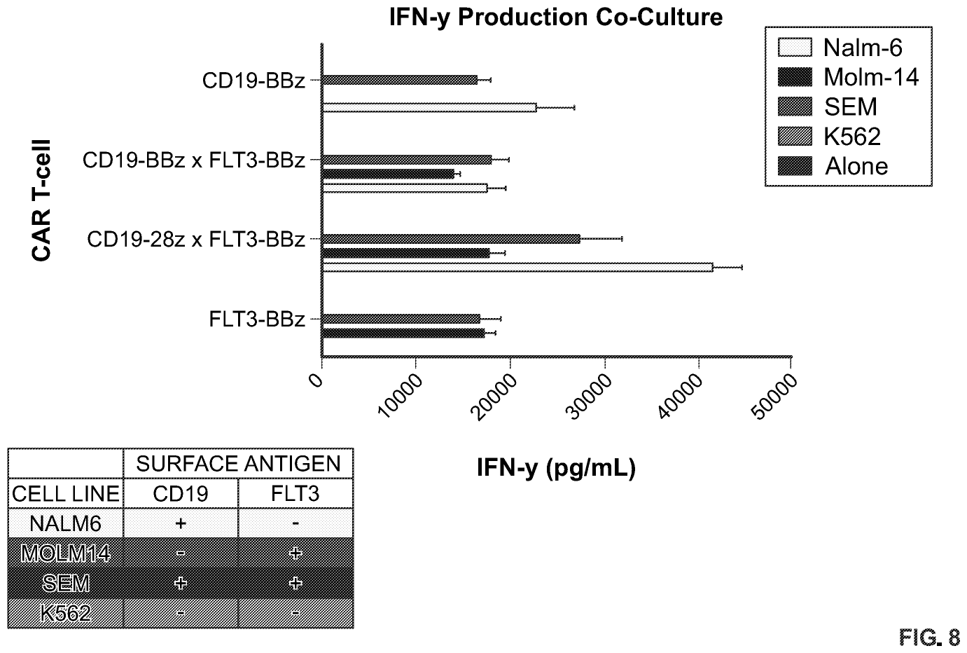

T-cells were transfected with either an expression vector comprising the nucleic acid molecule put forth in Embodiment 48 (hereafter “CD19-28z×FLT3-BBz”) or an expression vector comprising the nucleic acid molecule put forth in Embodiment 56 (hereafter “CD19-BBz×FLT3-BBz”). The T-cells were then analyzed using Fluorescence-activated cell sorting (FACS) to determine if each of the CAR individual CAR polypeptides encoded by the expression vectors were expressed on the surface of the T-cells. The results of the FACs analysis are shown in FIG. 6. As shown in FIG. 6, each of the CAR constructs encoded by the expression vectors were expressed on the surface of the T-Cells. That is, the T-cells transfected with the expression vector comprising the nucleic acid molecule put forth in Embodiment 48 expressed on their cell surface a pair of CAR constructs corresponding to the pair of CAR constructs described in Embodiments 49 and 50, and the T-cells transfected with the expression vector comprising the nucleic acid molecule put forth in Embodiment 56 expressed on their cell surface a pair of CAR constructs corresponding to the pair of CAR constructs described in Embodiments 57 and 58.

Accordingly, these results demonstrate that the bicistronic CAR constructs of the present disclosure can be expressed in T-cells. As would be appreciated by the skilled artisan, the methods used in this example can be used to test an expression vector comprising any of the nucleic acid molecules disclosed herein that encode for a bicistronic CAR construct or a bivalent CAR construct.

### Example 2—T-Cells Expressing Bicistronic CAR Constructs of the Present Disclosure are Stimulated in the Presence of One or Two of the Corresponding Antigens

The following is a non-limiting example that demonstrates that T-cells expressing bicistronic CAR constructs of the present disclosure are stimulated and secrete IL-2 and IFN-γ in the presence of cells that express one or two of the corresponding antigens.

In this example, 4 different populations of T-cells were tested:
Population #1 (hereafter “CD19-BBz”): T-cells expressing an anti-CD19 CAR with a CD8a hinge domain, a CD8a transmembrane domain, a 4-1BB intracellular signaling sequence, and a CD3ζ intracellular T cell signaling sequence
Population #2 (hereafter “CD19-BBz×FLT3-BBz”): T-cells expressing a bicistronic CAR construct as put forth in Embodiments 57 and 58, wherein one of the CARs is an anti-CD19 CAR with a a CD8a hinge domain, a CD8a transmembrane domain, a 4-1BB intracellular signaling sequence, and a CD3ζ intracellular T cell signaling sequence and the other CAR is an anti-FLT3 CAR with a a CD8a hinge domain, a CD8a transmembrane domain, a 4-1BB intracellular signaling sequence, and a CD3ζ intracellular T cell signaling sequence
Population #3 (hereafter “CD19-28z×FLT3-BBz”): T-cells expressing a bicistronic CAR construct as put forth in Embodiments 49 and 50, wherein one of the CARs is an anti-CD19 CAR with a a CD28 hinge domain, a CD28 transmembrane domain, a CD28 intracellular signaling sequence, and a CD3ζ intracellular T cell signaling sequence and the other CAR is an anti-FLT3 CAR with a a CD8a hinge domain, a CD8a transmembrane domain, a 4-1BB intracellular signaling sequence, and a CD3ζ intracellular T cell signaling sequence
Population #4 (hereafter “FLT3-BBz”): T-cells expressing an anti-FLT3 CAR with a CD8a hinge domain, a CD8a transmembrane domain, a 4-1BB intracellular signaling sequence, and a CD3ζ intracellular T cell signaling sequence

Each of the four T-cell populations were independently incubated with:
a) NALM6 cells, which are B cell precursor leukemia cells that express CD19 and that do not express FLT3;
b) MOLM14 cells, which are acute myeloid leukemia cells that do not express CD19 and that express FLT3;
c) SEM cells, which are acute lymphoblastic leukaemia cells that express both CD19 and FLT3; and
d) K562 cells, which are myelogenous leukemia cells that do not express CD19 and FLT3.

After incubation, the amounts of IL-2 and IFN-γ in the cultures were measured. The results are shown in FIGS. 7 and 8, respectively. As shown in FIGS. 7 and 8, the CD19-BBz×FLT3-BBz and CD19-BBz×FLT3-BBz T-cells secreted IL-2 and IFN-γ in the presence of NALM6, MOLM14 and SEM cells, indicating that only one of CD19 and FLT3 need to be present on a cancer cell for effective targeting by the CD19-BBz×FLT3-BBz and CD19-BBz×FLT3-BBz cells.

Thus, the results summarized in this example demonstrate that the bicistronic CAR constructs of the present disclosure can more effectively target cancer cells than existing CAR constructs by targeting distinct populations of cancer cells that exhibit unique cell surface marker profiles. As would be appreciated by the skilled artisan, the methods used in this example can be used to test any of the bicistronic CAR constructs or a bivalent CAR constructs disclosed herein.

### Example 3—T-Cells Expressing Bicistronic CAR Constructs of the Present Disclosure Effectively Treat Cancer In Vivo

The following is a non-limiting example that demonstrates that the bicistronic CAR constructs of the present disclosure can be used to effectively treat cancer in vivo.

Mice were injected with 1E6 of SEM cells expressing luciferase. 6 days after injection of the SEM cells, mice were treated with one of the following treatments:
Treatment #1 (hereafter “Saline”): Saline (negative control)
Treatment #2 (hereafter “Mock”): Mock Treatment (negative control)
Treatment #3 (hereafter “10e6 CD19 (28z)×FLT3CART BBz”): 10E6 T-cells expressing a bicistronic CAR construct as put forth in Embodiments 49 and 50,
wherein one of the CARs is an anti-CD19 CAR with a a CD28 hinge domain, a CD28 transmembrane domain, a CD28 intracellular signaling sequence, and a CD3ζ intracellular T cell signaling sequence and the other CAR is an anti-FLT3 CAR with a a CD8a hinge domain, a CD8a transmembrane domain, a 4-1BB intracellular signaling sequence, and a CD3ζ intracellular T cell signaling sequence
Treatment #4 (hereafter “10e6 CD19 (BBz)×FLT3CART BBz”): 10E6 T-cells expressing a bicistronic CAR construct as put forth in Embodiments 57 and 58,
wherein one of the CARs is an anti-CD19 CAR with a a CD8a hinge domain, a CD8a transmembrane domain, a 4-1BB intracellular signaling sequence, and a CD3ζ intracellular T cell signaling sequence and the other CAR is an anti-FLT3 CAR with a a CD8a hinge domain, a CD8a transmembrane domain, a 4-1BB intracellular signaling sequence, and a CD3ζ intracellular T cell signaling sequence
Treatment #5 (hereafter “10e6 CD19 CART”): 10E6 T-cells expressing an anti-CD19 CAR
Treatment #6 (hereafter “10e6 FLT3CART oPRE”): 10E6 T-cells expressing an anti-FLT3 CAR regulated by an optimized and truncated version of woodchuck hepatitis virus posttranslational regulatory element (oPRE)
Treatment #7 (hereafter “10e6 FLT3CART WPRE”): 10E6 T-cells expressing an anti-FLT3 CAR regulated by a woodchuck hepatitis virus posttranslational regulatory element (WPRE)

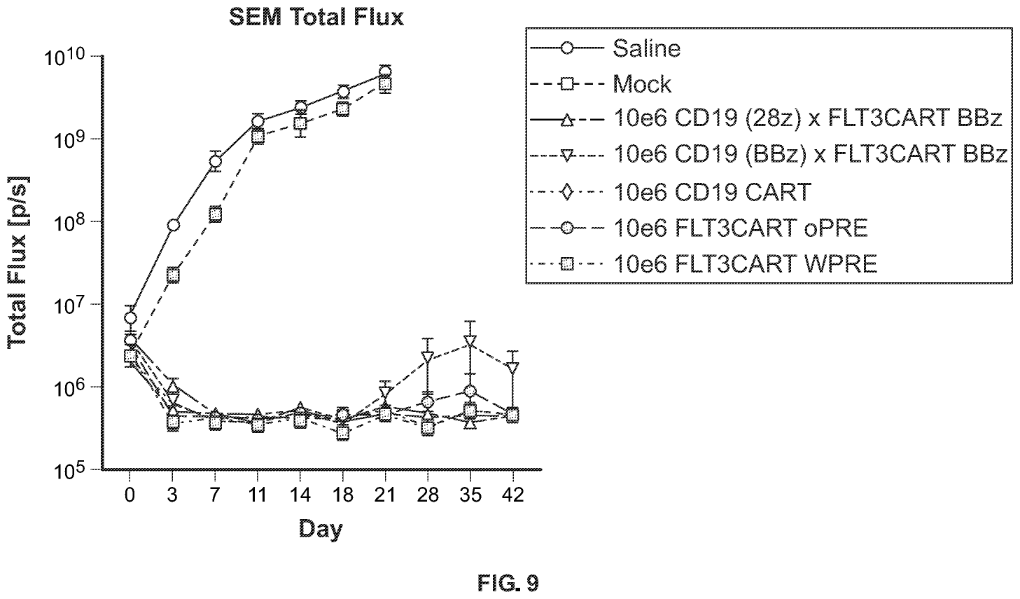

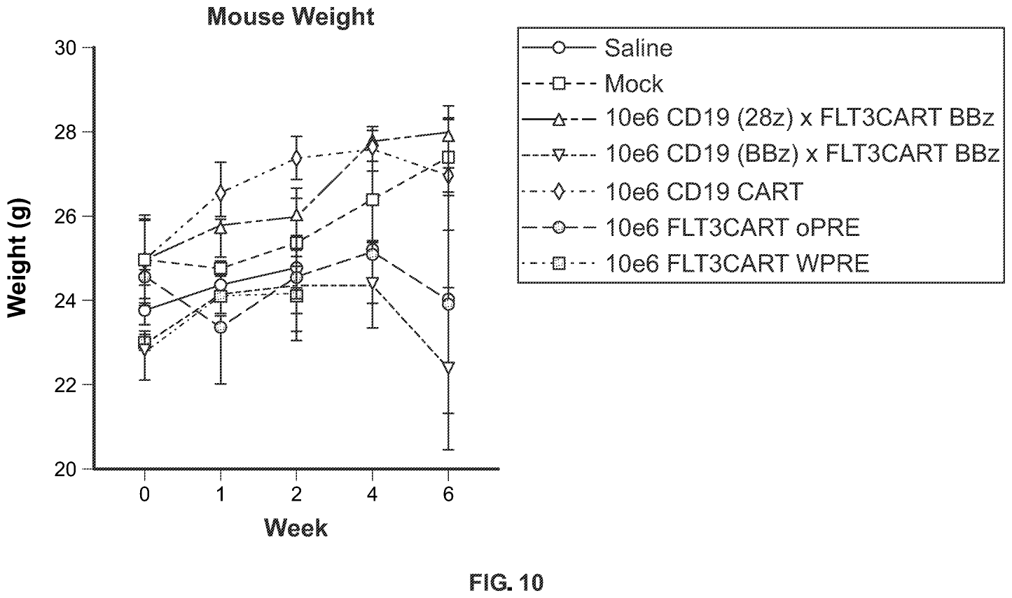

Luciferase signal was then recorded in the mice at days 3, 7, 11, 14, 18, 21, 28, 35 and 42 following treatment to track SEM tumor growth. The results of this analysis are shown in FIG. 9. The results shown in FIG. 9 that the CD19 (BBz)×FLT3CART BBz T-cells and CD19 (28z)×FLT3CART BBz T-cells eliminated SEM tumor growth in the mice as compared to controls. The weight of the mice was also tracked following treatment, and the results are shown in FIG. 10. The results shown in FIG. 10 demonstrate that the CD19 (BBz)×FLT3CART BBz T-cells and CD19 (28z)×FLT3CART BBz T-cells were well tolerated by the mice.

Thus, the results summarized in this example demonstrate that the bicistronic CAR constructs of the present disclosure can effectively treat cancer in vivo. As would be appreciated by the skilled artisan, the methods used in this example can be used to test any of the bicistronic CAR constructs or a bivalent CAR constructs disclosed herein.

### Example 4—T-Cells Expressing Bicistronic CAR Constructs of the Present Disclosure Effectively Treat Cancer In Vivo

The following is a non-limiting example that demonstrates that the bicistronic CAR constructs of the present disclosure can be used to effectively treat cancer in vivo.

Mice were injected with 1E6 of MOLM14 cells expressing luciferase. 3 days after injection of the SEM cells, mice were treated with one of the following treatments:
Treatment #1 (hereafter “UTD 15e6”): Negative Control
Treatment #2 (hereafter “UTD ZTG 15e6”): Negative Control
Treatment #3 (hereafter “FLT3 ZTG LV 15e6”): 15E6 T-cells expressing an anti-FLT3 CAR
Treatment #4 (hereafter “1928z×FLT3-BBz 5e6”): 5E6 T-cells expressing a bicistronic CAR construct as put forth in Embodiments 49 and 50, wherein one of the CARs is an anti-CD19 CAR with a a CD28 hinge domain, a CD28 transmembrane domain, a CD28 intracellular signaling sequence, and a CD3ζ intracellular T cell signaling sequence and the other CAR is an anti-FLT3 CAR with a a CD8a hinge domain, a CD8a transmembrane domain, a 4-1BB intracellular signaling sequence, and a CD3ζ intracellular T cell signaling sequence
Treatment #5 (hereafter “1928z×FLT3-BBz 10e6”): 10E6 T-cells expressing a bicistronic CAR construct as put forth in Embodiments 49 and 50, wherein one of the CARs is an anti-CD19 CAR with a a CD28 hinge domain, a CD28 transmembrane domain, a CD28 intracellular signaling sequence, and a CD3ζ intracellular T cell signaling sequence and the other CAR is an anti-FLT3 CAR with a a CD8a hinge domain, a CD8a transmembrane domain, a 4-1BB intracellular signaling sequence, and a CD3ζ intracellular T cell signaling sequence
Treatment #6 (hereafter “19-BBz×FLT3-BBz 5e6”): 5E6 T-cells expressing a bicistronic CAR construct as put forth in Embodiments 57 and 58, wherein one of the CARs is an anti-CD19 CAR with a a CD8a hinge domain, a CD8a transmembrane domain, a 4-1BB intracellular signaling sequence, and a CD3ζ intracellular T cell signaling sequence and the other CAR is an anti-FLT3 CAR with a a CD8a hinge domain, a CD8a transmembrane domain, a 4-1BB intracellular signaling sequence, and a CD3ζ intracellular T cell signaling sequence
Treatment #7 (hereafter “19-BBz×FLT3-BBz 10e6”): 10E6 T-cells expressing a bicistronic CAR construct as put forth in Embodiments 57 and 58, wherein one of the CARs is an anti-CD19 CAR with a a CD8a hinge domain, a CD8a transmembrane domain, a 4-1BB intracellular signaling sequence, and a CD3ζ intracellular T cell signaling sequence and the other CAR is an anti-FLT3 CAR with a a CD8a hinge domain, a CD8a transmembrane domain, a 4-1BB intracellular signaling sequence, and a CD3ζ intracellular T cell signaling sequence

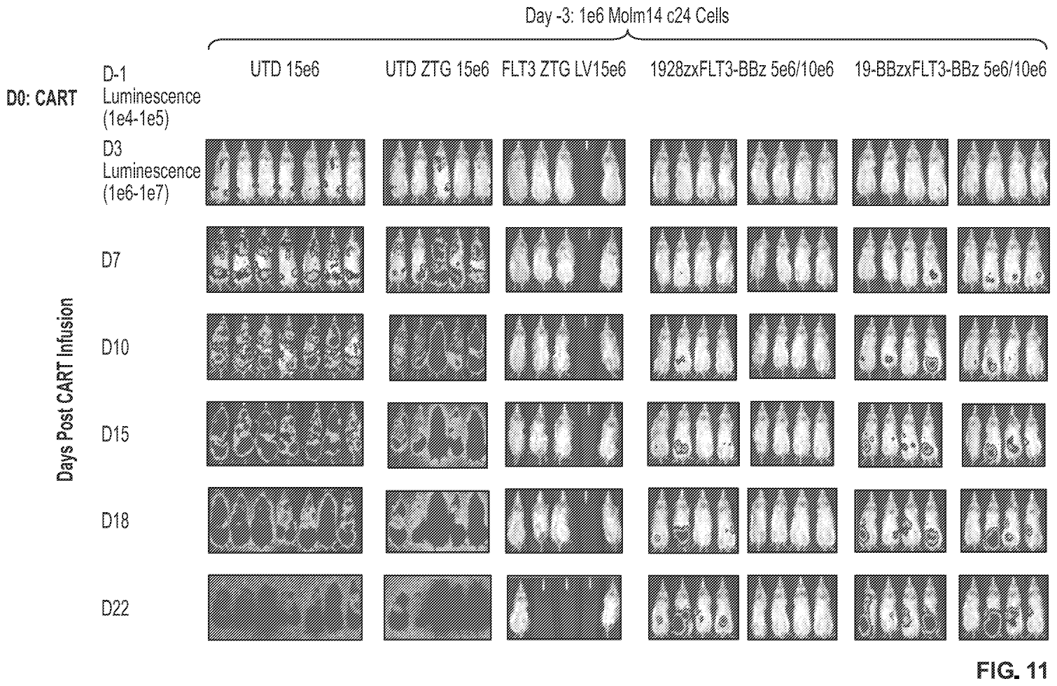

Luciferase signal was then recorded in the mice at days 3, 7, 10, 15, 18 and 22 following treatment to track MOLM14 tumor growth. The results of this analysis are shown in FIG. 11. The results shown in FIG. 9 that the 19-BBz×FLT3-BBz T-cells and 1928z×FLT3-BBz T-cells attenuated and, in some cases, eliminated MOLM14 tumor growth in the mice as compared to controls.

Thus, the results summarized in this example demonstrate that the bicistronic CAR constructs of the present disclosure can effectively treat cancer in vivo. As would be appreciated by the skilled artisan, the methods used in this example can be used to test any of the bicistronic CAR constructs or a bivalent CAR constructs disclosed herein.

What is claimed is:

1. A composition comprising a first polypeptide and a second polypeptide,
wherein the first polypeptide comprises:
a) an scFv that binds to CD19;
b) a CD28 hinge region;
c) a CD28 transmembrane domain;
d) a CD28 intracellular signaling sequence;
e) a CD3ζ intracellular T cell signaling sequence; and
wherein the second polypeptide comprises
a) an scFv that binds to FLT3;
b) a CD8a hinge domain;
c) a CD8a transmembrane domain;
d) a 4-1BB intracellular signaling sequence; and
e) a CD3ζ intracellular T cell signaling sequence.

2. A composition comprising a first polypeptide and a second polypeptide,
wherein the first polypeptide comprises:
a) a first leader sequence;
b) an scFv that binds to CD19;
c) a CD8a hinge domain;
d) a CD8a transmembrane domain;
e) a 4-1BB intracellular signaling sequence; and
f) a CD3ζ intracellular T cell signaling sequence; and
wherein the second polypeptide comprises:
a) a second leader sequence;
b) an scFv that binds to FLT3;
c) a CD8a hinge domain;
d) a CD8a transmembrane domain;
e) a 4-1BB intracellular signaling sequence; and
f) a CD3ζ intracellular T cell signaling sequence.

3. The composition of any one of the preceding claims, wherein the scFv that binds to CD19 comprises the amino acid sequence put forth in SEQ ID NO: 33, the amino acid sequence put forth in SEQ ID NO: 67 and the amino acid sequence put forth in SEQ ID NO: 34.

4. The composition of any one of the preceding claims, wherein the scFv that binds to CD19 comprises the amino acid sequence put forth in SEQ ID NO: 121.

5. The composition of any one of the preceding claims, wherein the CD28 hinge region comprises the amino acid sequence put forth in SEQ ID NO: 122.

6. The composition of any one of the preceding claims, wherein the CD28 transmembrane domain comprises the amino acid sequence put forth in SEQ ID NO: 79.

7. The composition of any one of the preceding claims, wherein the CD28 intracellular signaling sequence comprises the amino acid sequence put forth in SEQ ID NO: 123.

8. The composition of any one of the preceding claims, wherein the CD3ζ intracellular T cell signaling sequence comprises amino acid sequence put forth in SEQ ID NO: 74.

9. The composition of any one of the preceding claims, wherein the CD3ζ intracellular T cell signaling sequence comprises amino acid sequence put forth in SEQ ID NO: 124.

10. The composition of any one of the preceding claims, wherein the scFv that binds to FLT3 comprises the amino acid sequence put forth in SEQ ID NO: 18.

11. The composition of any one of the preceding claims, wherein the scFv that binds to FLT3 comprises the amino acid sequence put forth in SEQ ID NO: 127.

12. The composition of any one of the preceding claims, wherein the CD8a hinge domain comprises the amino acid sequence put forth in SEQ ID NO: 72.

13. The composition of any one of the preceding claims, wherein the CD8a transmembrane domain comprises the amino acid sequence put forth in SEQ ID NO: 71.

14. The composition of any one of the preceding claims, wherein the 4-1BB intracellular signaling sequence comprises the amino acid sequence put forth in SEQ ID NO: 73.

15. The composition of any one of the preceding claims,
wherein the first polypeptide comprises:
a) an scFv that binds to CD19, wherein the scFv that binds to CD19 comprises the amino acid sequence put forth in SEQ ID NO: 121;
b) a CD28 hinge region, wherein the CD28 hinge region comprises the amino acid sequence put forth in SEQ ID NO: 122;
c) a CD28 transmembrane domain, wherein the CD28 transmembrane domain comprises the amino acid sequence put forth in SEQ ID NO: 79;
d) a CD28 intracellular signaling sequence, wherein the CD28 intracellular signaling sequence comprises the amino acid sequence put forth in SEQ ID NO: 123;
e) a CD3ζ intracellular T cell signaling sequence, wherein the CD3ζ intracellular T cell signaling sequence comprises amino acid sequence put forth in SEQ ID NO: 124; and
wherein the second polypeptide comprises:
a) an scFv that binds to FLT3, wherein the scFv that binds to FLT3 comprises the amino acid sequence put forth in SEQ ID NO: 18;
b) a CD8a hinge domain, wherein the CD8a hinge domain comprises the amino acid sequence put forth in SEQ ID NO: 72;
c) a CD8a transmembrane domain, wherein the CD8a transmembrane domain comprises the amino acid sequence put forth in SEQ ID NO: 71;
d) a 4-1BB intracellular signaling sequence, wherein the 4-1BB intracellular signaling sequence comprises the amino acid sequence put forth in SEQ ID NO: 73; and
e) a CD3ζ intracellular T cell signaling sequence, wherein the CD3ζ intracellular T cell signaling sequence comprises amino acid sequence put forth in SEQ ID NO: 124.

16. A composition of any one of the preceding claims,
wherein the first polypeptide comprises:
a) an scFv that binds to CD19, wherein the scFv that binds to CD19 comprises the amino acid sequence put forth in SEQ ID NO: 121;
b) a CD8a hinge domain, wherein the CD8a hinge domain comprises the amino acid sequence put forth in SEQ ID NO: 72;
c) a CD8a transmembrane domain, wherein the CD8a transmembrane domain comprises the amino acid sequence put forth in SEQ ID NO: 71;
d) a 4-1BB intracellular signaling sequence, wherein the 4-1BB intracellular signaling sequence comprises the amino acid sequence put forth in SEQ ID NO: 73;
e) a CD3ζ intracellular T cell signaling sequence, wherein the CD3ζ intracellular T cell signaling sequence comprises amino acid sequence put forth in SEQ ID NO: 124;
and
wherein the second polypeptide comprises:
a) an scFv that binds to FLT3 wherein the scFv that binds to FLT3 comprises the amino acid sequence put forth in SEQ ID NO: 18;
b) a CD8a hinge domain, wherein the CD8a hinge domain comprises the amino acid sequence put forth in SEQ ID NO: 72;
c) a CD8a transmembrane domain, wherein the CD8a transmembrane domain comprises the amino acid sequence put forth in SEQ ID NO: 71;
d) a 4-1BB intracellular signaling sequence, wherein the 4-1BB intracellular signaling sequence comprises the amino acid sequence put forth in SEQ ID NO: 73; and
e) a CD3ζ intracellular T cell signaling sequence, wherein the CD3ζ intracellular T cell signaling sequence comprises amino acid sequence put forth in SEQ ID NO: 124.

17. The composition of any one of the preceding claims, wherein the first polypeptide comprises the amino acid sequence put forth in SEQ ID NO: 141 and the second polypeptide comprises the amino acid sequence put forth in SEQ ID NO: 142.

18. The composition of any one of the preceding claims, wherein the first polypeptide comprises the amino acid sequence put forth in SEQ ID NO: 151 and the second polypeptide comprises the amino acid sequence put forth in SEQ ID NO: 152.

19. An expression construct comprising a nucleotide sequence encoding the first polypeptide and the second polypeptide of the composition of any one of the preceding claims, wherein the nucleotide sequence encoding the first polypeptide is joined to a nucleotide sequence encoding the second polypeptide by a nucleic acid sequence that encodes a self-cleaving linker peptide.

20. A host cell comprising the composition or expression construct of any one of the preceding claims.

21. The host cell of claim 20, wherein the host cell is a T-cell.

22. A population of cells comprising at least one host cell of claim 20 or claim 21.

23. A method of treating cancer in a subject, the method comprising administering to the subject an effective amount of host cells according to claim 20 or claim 21.

24. The method of claim 23, wherien the cancer is acute lymphoblastic leukemia (ALL) or acute myeloid leukemia (AML) in a subject

25. The method of claim 23 or claim 24, wherein the effective amount of host cells is at least 1×10⁶ cells.

26. The method of any one of claims 24-25, wherein the ALL is B-cell ALL (B-ALL).

27. The host cell of claim 20 or claim 21 for use in the treatment of cancer in a subject.

28. The use of claim 27, wherien the cancer is acute lymphoblastic leukemia (ALL) or acute myeloid leukemia (AML) in a subject

29. The use of claim 28, wherein the ALL is B-cell ALL (B-ALL).

30. A pair of chimeric antigen receptors (CAR) comprising:
a first CAR comprising:
an anti-FLT3 antigen binding domain;
a transmembrane domain; and
an intracellular T-cell signaling domain; and
a second CAR comprising:
an antigen binding domain selected from the group consisting of an anti-CD19 antigen binding domain; an anti-CD22 antigen binding domain; an anti-CD33 antigen binding domain and an anti-CD123 antigen binding domain;
a transmembrane domain; and
an intracellular T-cell signaling domain.

31. An expression construct comprising a nucleotide sequence encoding the pair of CARs according to claim 30, wherein a nucleotide sequence encoding the first CAR is joined to a nucleotide sequence encoding the second CAR by a nucleotide that encodes a self-cleaving linker peptide.

32. A recombinant expression vector comprising the expression construct of claim 31.

33. A host cell comprising the pair of CARs of claim 30 and/or the recombinant expression vector of claim 31.

34. The host cell of claim 33, wherein the host cell is a T-cell.

35. A population of cells comprising at least one host cell of claim 33 or claim 34.

36. A method of treating acute lymphoblastic leukemia (ALL) or acute myeloid leukemia (AML) in a subject, the method comprising administering to the subject an effective amount of host cells according to claim 33 or claim 34.

37. The method of claim 36, wherein the effective amount of host cells is at least 1×10⁶ cells.

38. The method of claim 36 or claim 37, wherein the ALL is B-cell ALL (B-ALL).

39. The pair of CARs of any one of claims 29-39 for use in the treatment of acute lymphoblastic leukemia (ALL) or acute myeloid leukemia (AML).

40. The pair of CARs of any one of the preceding claims, wherein the CARs are expressed on the surface of a T-cell.


SEQUENCE LISTING (157 sequences)

SEQ ID NO: 1 - protein, 25 aa, Artificial Sequence
  Synthetic Sequence
EVQLVQSGAEVKKPGSSVKVSCKAS

SEQ ID NO: 2 - protein, 10 aa, Artificial Sequence
  Synthetic Sequence
GGTFSSYAIS

SEQ ID NO: 3 - protein, 14 aa, Artificial Sequence
  Synthetic Sequence
WVRQAPGQGLEWMG

SEQ ID NO: 4 - protein, 17 aa, Artificial Sequence
  Synthetic Sequence
GIIPIFGTANYAQKFQG

SEQ ID NO: 5 - protein, 32 aa, Artificial Sequence
  Synthetic Sequence
RVTITADKSTSTAYMELSSLRSEDTAVYYCAT

SEQ ID NO: 6 - protein, 13 aa, Artificial Sequence
  Synthetic Sequence
FALFGFREQAFDI

SEQ ID NO: 7 - protein, 11 aa, Artificial Sequence
  Synthetic Sequence
WGQGTTVTVSS

SEQ ID NO: 8 - protein, 15 aa, Artificial Sequence
  Artificial Sequence
GGGGSGGGGSGGGGS

SEQ ID NO: 9 - protein, 23 aa, Artificial Sequence
  Synthetic Sequence
DIQMTQSPSSLSASVGDRVTITC

SEQ ID NO: 10 - protein, 11 aa, Artificial Sequence
  Synthetic Sequence
RASQSISSYLN

SEQ ID NO: 11 - protein, 15 aa, Artificial Sequence
  Synthetic Sequence
WYQQKPGKAPKLLIY

SEQ ID NO: 12 - protein, 7 aa, Artificial Sequence
  Synthetic Sequence
AASSLQS

SEQ ID NO: 13 - protein, 32 aa, Artificial Sequence
  Synthetic Sequence
GVPSRFSGSGSGTDFTLTISSLQPEDLATYYC

SEQ ID NO: 14 - protein, 9 aa, Artificial Sequence
  Synthetic Sequence
QQSYSTPFT

SEQ ID NO: 15 - protein, 10 aa, Artificial Sequence
  Synthetic Sequence
FGPGTKVDIK

SEQ ID NO: 16 - protein, 122 aa, Artificial Sequence
  Synthetic Sequence
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSS

SEQ ID NO: 17 - protein, 107 aa, Artificial Sequence
  Synthetic Sequence
DIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIK

SEQ ID NO: 18 - protein, 244 aa, Artificial Sequence
  Synthetic Sequence
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIK

SEQ ID NO: 19 - protein, 25 aa, Artificial Sequence
  Synthetic Sequence
EVKLQESGPGLVAPSQSLSVTCTVS

SEQ ID NO: 20 - protein, 8 aa, Artificial Sequence
  Synthetic Sequence
GVSLPDYG

SEQ ID NO: 21 - protein, 17 aa, Artificial Sequence
  Synthetic Sequence
VSWIRQPPRKGLEWLGV

SEQ ID NO: 22 - protein, 7 aa, Artificial Sequence
  Synthetic Sequence
IWGSETT

SEQ ID NO: 23 - protein, 38 aa, Artificial Sequence
  Synthetic Sequence
YYNSALKSRLTIIKDNSKSQVFLKMNSLQTDDTAIYYC

SEQ ID NO: 24 - protein, 14 aa, Artificial Sequence
  Synthetic Sequence
AKHYYYGGSYAMDY

SEQ ID NO: 25 - protein, 11 aa, Artificial Sequence
  Synthetic Sequence
WGQGTSVTVSS

SEQ ID NO: 26 - protein, 26 aa, Artificial Sequence
  Synthetic Sequence
DIQMTQTTSSLSASLGDRVTISCRAS

SEQ ID NO: 27 - protein, 6 aa, Artificial Sequence
  Synthetic Sequence
QDISKY

SEQ ID NO: 28 - protein, 17 aa, Artificial Sequence
  Synthetic Sequence
LNWYQQKPDGTVKLLIY

SEQ ID NO: 29 - protein, 3 aa, Artificial Sequence
  Synthetic Sequence
HTS

SEQ ID NO: 30 - protein, 36 aa, Artificial Sequence
  Synthetic Sequence
RLHSGVPSRFSGSGSGTDYSLTISNLEQEDIATYFC

SEQ ID NO: 31 - protein, 9 aa, Artificial Sequence
  Synthetic Sequence
QQGNTLPYT

SEQ ID NO: 32 - protein, 10 aa, Artificial Sequence
  Synthetic Sequence
FGGGTKLEIT

SEQ ID NO: 33 - protein, 120 aa, Artificial Sequence
  Synthetic Sequence
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 34 - protein, 107 aa, Artificial Sequence
  Synthetic Sequence
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 35 - protein, 25 aa, Artificial Sequence
  Synthetic Sequence
QVQLQQSGPGLVKPSQTLSLTCAIS

SEQ ID NO: 36 - protein, 10 aa, Artificial Sequence
  Synthetic Sequence
GDSVSSNSAA

SEQ ID NO: 37 - protein, 17 aa, Artificial Sequence
  Synthetic Sequence
WNWIRQSPSRGLEWLGR

SEQ ID NO: 38 - protein, 9 aa, Artificial Sequence
  Synthetic Sequence
TYYRSKWYN

SEQ ID NO: 39 - protein, 39 aa, Artificial Sequence
  Synthetic Sequence
NDYAVSVKSRITINPDTSKNQFSLQLNSVTPEDTAVYYC

SEQ ID NO: 40 - protein, 14 aa, Artificial Sequence
  Synthetic Sequence
AREVTGDLEDAFDI

SEQ ID NO: 41 - protein, 11 aa, Artificial Sequence
  Synthetic Sequence
WGQGTMVTVSS

SEQ ID NO: 42 - protein, 26 aa, Artificial Sequence [sequence omitted]

SEQ ID NO: 43 - protein, 6 aa, Artificial Sequence
  Synthetic Sequence
QTIWSY

SEQ ID NO: 44 - protein, 17 aa, Artificial Sequence
  Synthetic Sequence
LNWYQQRPGKAPNLLIY

SEQ ID NO: 45 - protein, 3 aa, Artificial Sequence
  Synthetic Sequence
AAS

SEQ ID NO: 46 - protein, 36 aa, Artificial Sequence
  Synthetic Sequence
SLQSGVPSRFSGRGSGTDFTLTISSLQAEDFATYYC

SEQ ID NO: 47 - protein, 9 aa, Artificial Sequence
  Synthetic Sequence
QQSYSIPQT

SEQ ID NO: 48 - protein, 10 aa, Artificial Sequence
  Synthetic Sequence
FGQGTKLEIK

SEQ ID NO: 49 - protein, 124 aa, Artificial Sequence
  Synthetic Sequence
QVQLQQSGPGLVKPSQTLSLTCAISGDSVSSNSAAWNWIRQSPSRGLEWLGRTYYRSKWYNDYAVSVKSRLTINPDTSKNQFSLQLNSVTPEDTAVYYCAREVTGDLEDAFDIWGQGTMVTVSS

SEQ ID NO: 50 - protein, 107 aa, Artificial Sequence
  Synthetic Sequence
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 51 - protein, 116 aa, Artificial Sequence
  Synthetic Sequence
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 52 - protein, 111 aa, Artificial Sequence
  Synthetic Sequence
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 53 - protein, 116 aa, Artificial Sequence
  Synthetic Sequence
EVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVSS

SEQ ID NO: 54 - protein, 111 aa, Artificial Sequence
  Synthetic Sequence
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIK

SEQ ID NO: 55 - protein, 141 aa, Artificial Sequence
  Synthetic Sequence
MEWSWVFLFFLSVTTGVHSEVQLVQSGAEVKKPGSSVKVSCKASGYTITDSNIHWVRQAPGQSLEWIGYIYPYNGGTDYNQKFKNRATLTVDNPTNTAYMELSSLRSEDTDFYYCVNGNPWLAYWGQGTLVTVSSASTKGP

SEQ ID NO: 56 - protein, 133 aa, Artificial Sequence
  Synthetic Sequence
MSVPTQVLGLLLLWLTDARCDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRT

SEQ ID NO: 57 - protein, 21 aa, Artificial Sequence
  Synthetic Sequence
GSGEGRGSLLTCGDVEENPGP

SEQ ID NO: 58 - protein, 22 aa, Artificial Sequence
  Synthetic Sequence
GSGATNFSLLKQAGDVEENPGP

SEQ ID NO: 59 - protein, 23 aa, Artificial Sequence
  Synthetic Sequence
GSGQCTNYALLKLAGDVESNPGP

SEQ ID NO: 60 - protein, 25 aa, Artificial Sequence
  Synthetic Sequence
GSGVKQTLNFDLLKLAGDVESNPGP

SEQ ID NO: 61 - protein, 10 aa, Artificial Sequence
  Synthetic Sequence
GGGGSGGGGS

SEQ ID NO: 62 - protein, 15 aa, Artificial Sequence
  Synthetic Sequence
GGGGSGGGGSGGGGS

SEQ ID NO: 63 - protein, 20 aa, Artificial Sequence
  Synthetic Sequence
GGGGSGGGGSGGGGSGGGGS

SEQ ID NO: 64 - protein, 30 aa, Artificial Sequence
  Synthetic Sequence
GGGGSGGGGSGGGGSGGGGSGGGGSGGGGS

SEQ ID NO: 65 - protein, 35 aa, Artificial Sequence
  Synthetic Sequence
GGGGSGGGGSGGGGSGGGGSGGGGSGGGGSGGGGS

SEQ ID NO: 66 - protein, 40 aa, Artificial Sequence
  Synthetic Sequence
GGGGSGGGGSGGGGSGGGGSGGGGSGGGGSGGGGSGGGGS

SEQ ID NO: 67 - protein, 18 aa, Artificial Sequence
  Synthetic Sequence
GSTSGSGKPGSGEGSTKG

SEQ ID NO: 68 - protein, 5 aa, Artificial Sequence
  Synthetic Sequence
GGGGS

SEQ ID NO: 69 - protein, 25 aa, Artificial Sequence
  Synthetic Sequence
GGGGSGGGGSGGGGSGGGGSGGGGS

SEQ ID NO: 70 - protein, 21 aa, Artificial Sequence
  Synthetic Sequence
LLLVTSLLLCELPHPAFLLIP

SEQ ID NO: 71 - protein, 24 aa, Artificial Sequence
  Synthetic Sequence
IYIWAPLAGTCGVLLLSLVITLYC

SEQ ID NO: 72 - protein, 45 aa, Artificial Sequence
  Synthetic Sequence
TTTPAPRPPTPAPTIASQPLSLRPEACRPAAGGAVHTRGLDFACD

SEQ ID NO: 73 - protein, 42 aa, Artificial Sequence
  Synthetic Sequence
KRGRKKLLYIFKQPFMRPVQTTQEEDGCSCRFPEEEEGGCEL

SEQ ID NO: 74 - protein, 112 aa, Artificial Sequence
  Synthetic Sequence
  MISC_FEATURE (14)..(14): either K or Q
RVKFSRSADAPAYXQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 18 aa, Artificial Sequence
  Synthetic Sequence
EGRGSLLTCGDVEENPGP

SEQ ID NO: 76 - protein, 19 aa, Artificial Sequence
  Synthetic Sequence
ATNFSLLKQAGDVEENPGP

SEQ ID NO: 77 - protein, 20 aa, Artificial Sequence
  Synthetic Sequence
QCTNYALLKLAGDVESNPGP

SEQ ID NO: 78 - protein, 22 aa, Artificial Sequence
  Synthetic Sequence
VKQTLNFDLLKLAGDVESNPGP

SEQ ID NO: 79 - protein, 27 aa, Artificial Sequence
  Synthetic Sequence
FWVLVVVGGVLACYSLLVTVAFIIFWV

SEQ ID NO: 80 - protein, 44 aa, Artificial Sequence
  Synthetic Sequence
TSAAAIEVMYPPPYLDNEKSNGTIIHVKGKHLCPSPLFPGPSKP

SEQ ID NO: 81 - protein, 42 aa, Artificial Sequence; identical to SEQ ID NO: 73
  Synthetic Sequence

SEQ ID NO: 82 - protein, 116 aa, Artificial Sequence
  Synthetic Sequence
EVQLVESGGGLVQPGGSLRLSCAASGFTFSSYGMHWVRQAPGKGLEWVAVLSYDGSNKYYADSVKGRFTISRDNSKNTLYLQMNSLRAEDTAVYYCANLAPWAAYWGQGTLVTVSS

SEQ ID NO: 83 - protein, 112 aa, Artificial Sequence
  Synthetic Sequence
EIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPHTFGQGTKLEIK

SEQ ID NO: 84 - protein, 243 aa, Artificial Sequence
  Synthetic Sequence
EVQLVESGGGLVQPGGSLRLSCAASGFTFSSYGMHWVRQAPGKGLEWVAVLSYDGSNKYYADSVKGRFTISRDNSKNTLYLQMNSLRAEDTAVYYCANLAPWAAYWGQGTLVTVSSGGGGSGGGGSGGGGSEIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPHTFGQGTKLEIK

SEQ ID NO: 85 - protein, 116 aa, Artificial Sequence
  Synthetic Sequence
EVQLVESGGGVVQPGGSLRLSCAASGFTFSSYGMHWVRQAPGKGLEWVAVISYDGSNKYYADSVKGRFTISRDNSKNTLYLQMNSLRAEDTAVYYCANLAPWAAYWGQGTLVTVSS

SEQ ID NO: 86 - protein, 112 aa, Artificial Sequence
  Synthetic Sequence
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 87 - protein, 243 aa, Artificial Sequence
  Synthetic Sequence
EVQLVESGGGVVQPGGSLRLSCAASGFTFSSYGMHWVRQAPGKGLEWVAVLSYDGSNKYYADSVKGRFTISRDNSKNTLYLQMNSLRAEDTAVYYCANLAPWAAYWGQGTLVTVSSGGGGSGGGGSGGGGSDVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 88 - protein, 115 aa, Artificial Sequence
  Synthetic Sequence
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSS

SEQ ID NO: 89 - protein, 5 aa, Artificial Sequence
  Synthetic Sequence
SYWMN

SEQ ID NO: 90 - protein, 17 aa, Artificial Sequence
  Synthetic Sequence
RIDPYDSETHYNQKFKD

SEQ ID NO: 91 - protein, 6 aa, Artificial Sequence
  Synthetic Sequence
GNWDDY

SEQ ID NO: 92 - protein, 107 aa, Artificial Sequence
  Synthetic Sequence
DVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIK

SEQ ID NO: 93 - protein, 11 aa, Artificial Sequence
  Synthetic Sequence
RASKSISKDLA

SEQ ID NO: 94 - protein, 7 aa, Artificial Sequence
  Synthetic Sequence
SGSTLQS

SEQ ID NO: 95 - protein, 9 aa, Artificial Sequence
  Synthetic Sequence
QQHNKYPYT

SEQ ID NO: 96 - protein, 118 aa, Artificial Sequence
  Synthetic Sequence
QIQLVQSGPELKKPGETVKISCKTSGYVFTNYGMNWVKQAPGKGFKWMGWMNTNTGEPTSLEDFKGRFAFSLETSASTAYLQINNLKNDDTATYFCARSGGYDPMDYWGQGTSVTVSS

SEQ ID NO: 97 - protein, 5 aa, Artificial Sequence
  Synthetic Sequence
NYGMN

SEQ ID NO: 98 - protein, 17 aa, Artificial Sequence
  Synthetic Sequence
WMNTNTGEPTSLEDFKG

SEQ ID NO: 99 - protein, 9 aa, Artificial Sequence
  Synthetic Sequence
SGGYDPMDY

SEQ ID NO: 100 - protein, 110 aa, Artificial Sequence
  Synthetic Sequence
DIVLTQSPASLAVSPGQRATISCRASESVDNYGNTFMHWYQQKPGQPPKLLIYRASNLESGIPARFSGSDSRTDFTLTINPVEADDVATYYCQQSKEDPPTFGGTKLELK

SEQ ID NO: 101 - protein, 15 aa, Artificial Sequence
  Synthetic Sequence
RASESVDNYGNTFMH

SEQ ID NO: 102 - protein, 7 aa, Artificial Sequence
  Synthetic Sequence
RASNLES

SEQ ID NO: 103 - protein, 9 aa, Artificial Sequence
  Synthetic Sequence
QQSKEDPPT

SEQ ID NO: 104 - protein, 118 aa, Artificial Sequence
  Synthetic Sequence
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSS

SEQ ID NO: 105 - protein, 5 aa, Artificial Sequence
  Synthetic Sequence
NYGMN

SEQ ID NO: 106 - protein, 17 aa, Artificial Sequence
  Synthetic Sequence
WINTYTGESTYSADFKG

SEQ ID NO: 107 - protein, 9 aa, Artificial Sequence
  Synthetic Sequence
SGGYDPMDY

SEQ ID NO: 108 - protein, 110 aa, Artificial Sequence
  Synthetic Sequence
DIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKELK

SEQ ID NO: 109 - protein, 15 aa, Artificial Sequence
  Synthetic Sequence
RASESVDNYGNTFMH

SEQ ID NO: 110 - protein, 7 aa, Artificial Sequence
  Synthetic Sequence
RASNLES

SEQ ID NO: 111 - protein, 9 aa, Artificial Sequence
  Synthetic Sequence
QQSNEDPPT

SEQ ID NO: 112 - protein, 118 aa, Artificial Sequence
  Synthetic Sequence
QVQLQQPGAELVKPGAPVKLSCKASGYTFTNYWMNWIKQRPGRGLEWIGRIDPSDSESHYNQKFKDKATLTVDKSSNTAYIQLSSLTSEDSAVYYCARYDYDDTMDYWGQGTSVTVSS

SEQ ID NO: 113 - protein, 5 aa, Artificial Sequence
  Synthetic Sequence
NYWMN

SEQ ID NO: 114 - protein, 17 aa, Artificial Sequence
  Synthetic Sequence
RIDPSDSESHYNQKFKD

SEQ ID NO: 115 - protein, 9 aa, Artificial Sequence
  Synthetic Sequence
YDYDDTMDY

SEQ ID NO: 116 - protein, 112 aa, Artificial Sequence
  Synthetic Sequence
DIVMTQAAPSVPVTPGESVSISCRSNKSLLHSNGNTYLYWFLQRPGQSPQLLIYRMSNLASGVPDRFSGSGSGTAFTLRISRVEAEDVGVYYCMQHLEYPYTFGGGTKLEIK

SEQ ID NO: 117 - protein, 16 aa, Artificial Sequence
  Synthetic Sequence
RSNKSLLHSNGNTYLY

SEQ ID NO: 118 - protein, 7 aa, Artificial Sequence
  Synthetic Sequence
RMSNLAS

SEQ ID NO: 119 - protein, 9 aa, Artificial Sequence
  Synthetic Sequence
MQHLEYPYT

SEQ ID NO: 120 - protein, 21 aa, Artificial Sequence
  Synthetic Sequence
MEFGLSWLFLVAILKGVQCSR

SEQ ID NO: 121 - protein, 245 aa, Artificial Sequence
  Synthetic Sequence
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 122 - protein, 39 aa, Artificial Sequence
  Synthetic Sequence
IEVMYPPPYLDNEKSNGTIIHVKGKHLCPSPLFPGPSKP

SEQ ID NO: 123 - protein, 41 aa, Artificial Sequence
  Synthetic Sequence
RSKRSRLLHSDYMNMTPRRPGPTRKHYQPYAPPRDFAAYRS

SEQ ID NO: 124 - protein, 112 aa, Artificial Sequence
  Synthetic Sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 36 aa, Artificial Sequence
  Synthetic Sequence
RKRRGSGTPDPWGSGATNFSLLKQAGDVEENPGPLE

SEQ ID NO: 126 - protein, 21 aa, Artificial Sequence
  Synthetic Sequence
MLLVTSLLLCELPHPAFLLIP

SEQ ID NO: 127 - protein, 246 aa, Artificial Sequence
  Synthetic Sequence
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKSG

SEQ ID NO: 128 - DNA, 3066 bp, Artificial Sequence
  Synthetic Sequence
atggaattcggcctgagctggctgttcctggtggccattctgaagggcgtgcagtgcagcagagacatccagatgacccagaccaccagcagcctgtctgcctctctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggacattgccacctacttttgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttcttccggaatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagaagcgatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaggatccaggaagagaagaggctctggtacccccgatccttggggcagcggcgccacaaacttctctctgctaaagcaagcaggtgatgttgaagaaaaccccgggcctcttgagatgctgctggtcacatctctgctgctgtgcgagctgccccatcctgcctttctgctgattcctgaggtgcagctggtgcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacaagagcacaagcaccgcctacatggaactgagcagcctgagaagcgaggacaccgccgtgtactactgcgccacatttgccctgttcggcttcagagagcaggccttcgatatttggggccagggcaccaccgtgacagtttctagcggaggcggaggatctggtggcggaggaagtggcggaggcggtagcgatattcagatgacacagagccccagcagcctgtctgcctctgtgggagacagagtgacaattacctgccgggccagccagagcatcagctcctacctgaactggtatcagcaaaagcccggcaaggcccctaagctgctgatctatgctgcctccagtctgcagagcggcgtgccaagcagattttctggcagcggctctggcaccgacttcaccctgaccatatctagcctgcagccagaggacctggccacctactactgtcagcagagctacagcacccctttcaccttcggacctggcaccaaggtggacatcaagagcggcactagtaccaccacaccagctcctagacctccaactcctgctcctacaatcgccagccagcctctgtctctgaggccagaagcctgtagacctgctgctggcggagccgtgcatacaagaggactggatttcgcctgcgacatctacatctgggctcctctggccggaacatgcggagtgctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgtctagacgcgtgaagttctccagatccgctgatgctcccgcttatcagcaggggcagaatcagctctataatgaactgaatctggggcggagagaggaatacgacgtcctcgataagaggcgcggcagggaccctgagatgggaggaaaacctcggagaaaaaaccctcaggaagggctctacaacgagctgcagaaagataagatggctgaagcttactccgaaattgggatgaagggtgaaaggcggaggggcaaaggacacgacggactgtatcagggactgtccacagccacaaaagatacctatgacgcactccatatgcaggctctcccacccagatga

SEQ ID NO: 129 - DNA, 3077 bp, Artificial Sequence
  Synthetic Sequence
tcgagatggaattcggcctgagctggctgttcctggtggccattctgaagggcgtgcagtgcagcagagacatccagatgacccagaccaccagcagcctgtctgcctctctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggacattgccacctacttttgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttcttccggaatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagaagcgatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaggatccaggaagagaagaggctctggtacccccgatccttggggcagcggcgccacaaacttctctctgctaaagcaagcaggtgatgttgaagaaaaccccgggcctcttgagatgctgctggtcacatctctgctgctgtgcgagctgccccatcctgcctttctgctgattcctgaggtgcagctggtgcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacaagagcacaagcaccgcctacatggaactgagcagcctgagaagcgaggacaccgccgtgtactactgcgccacatttgccctgttcggcttcagagagcaggccttcgatatttggggccagggcaccaccgtgacagtttctagcggaggcggaggatctggtggcggaggaagtggcggaggcggtagcgatattcagatgacacagagccccagcagcctgtctgcctctgtgggagacagagtgacaattacctgccgggccagccagagcatcagctcctacctgaactggtatcagcaaaagcccggcaaggcccctaagctgctgatctatgctgcctccagtctgcagagcggcgtgccaagcagattttctggcagcggctctggcaccgacttcaccctgaccatatctagcctgcagccagaggacctggccacctactactgtcagcagagctacagcacccctttcaccttcggacctggcaccaaggtggacatcaagagcggcactagtaccaccacaccagctcctagacctccaactcctgctcctacaatcgccagccagcctctgtctctgaggccagaagcctgtagacctgctgctggcggagccgtgcatacaagaggactggatttcgcctgcgacatctacatctgggctcctctggccggaacatgcggagtgctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgtctagacgcgtgaagttctccagatccgctgatgctcccgcttatcagcaggggcagaatcagctctataatgaactgaatctggggcggagagaggaatacgacgtcctcgataagaggcgcggcagggaccctgagatgggaggaaaacctcggagaaaaaaccctcaggaagggctctacaacgagctgcagaaagataagatggctgaagcttactccgaaattgggatgaagggtgaaaggcggaggggcaaaggacacgacggactgtatcagggactgtccacagccacaaaagatacctatgacgcactccatatgcaggctctcccacccagatgagtcgac

SEQ ID NO: 130 - protein, 21 aa, Artificial Sequence
  Synthetic Sequence
MALPVTALLLPLALLLHAARP

SEQ ID NO: 131 - protein, 242 aa, Artificial Sequence
  Synthetic Sequence
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 132 - DNA, 3057 bp, Artificial Sequence
  Synthetic Sequence
atggctctgcctgttacagctctgctgctgcctctggctctgcttctgcatgctgctagacctcaggtgcagctggttcagtctggcgccgaagtgaagaaacctggcagcagcgtgaaggtgtcctgcaaggccagcggctacacctttaccgactacaacatgcactgggtccgacaggcccctggacaaggacttgagtggatcggctacatctacccctacaatggcggcaccggctacaaccagaagttcaagagcaaggccaccatcaccgccgacgagagcacaaacaccgcctacatggaactgagcagcctgagaagcgaggacaccgccgtgtactactgcgctagaggcagacccgccatggattattggggccagggaaccctggtcaccgtttctagcggaggcggaggatctggtggcggaggaagtggcggaggcggtagcgatattcagatgacacagagccccagcagcctgtctgccagcgtgggagatagagtgaccatcacctgtagagccagcgagagcgtggacaactacggcatcagcttcatgaactggttccagcagaagcccggcaaggcccctaagctgctgatctacgccgccagcaatcaaggcagcggagtgcctagcagattttccggctctggcagcggcaccgatttcaccctgaccatcagtagcctgcagcctgacgacttcgccacctactactgccagcagagcaaagaggtgccctggacatttggacagggcaccaaggtggaaatcaagtccggaatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagaagcgatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaggatccaggaagagaagaggctctggtacccccgatccttggggcagcggcgccacaaacttctctctgctaaagcaagcaggtgatgttgaagaaaaccccgggcctcttgagatgctgctggtcacatctctgctgctgtgcgagctgccccatcctgcctttctgctgattcctgaggtgcagctggtgcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacaagagcacaagcaccgcctacatggaactgagcagcctgagaagcgaggacaccgccgtgtactactgcgccacatttgccctgttcggcttcagagagcaggccttcgatatttggggccagggcaccaccgtgacagtttctagcggaggcggaggatctggtggcggaggaagtggcggaggcggtagcgatattcagatgacacagagccccagcagcctgtctgcctctgtgggagacagagtgacaattacctgccgggccagccagagcatcagctcctacctgaactggtatcagcaaaagcccggcaaggcccctaagctgctgatctatgctgcctccagtctgcagagcggcgtgccaagcagattttctggcagcggctctggcaccgacttcaccctgaccatatctagcctgcagccagaggacctggccacctactactgtcagcagagctacagcacccctttcaccttcggacctggcaccaaggtggacatcaagagcggcactagtaccaccacaccagctcctagacctccaactcctgctcctacaatcgccagccagcctctgtctctgaggccagaagcctgtagacctgctgctggcggagccgtgcatacaagaggactggatttcgcctgcgacatctacatctgggctcctctggccggaacatgcggagtgctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgtctagacgcgtgaagttctccagatccgctgatgctcccgcttatcagcaggggcagaatcagctctataatgaactgaatctggggcggagagaggaatacgacgtcctcgataagaggcgcggcagggaccctgagatgggaggaaaacctcggagaaaaaaccctcaggaagggctctacaacgagctgcagaaagataagatggctgaagcttactccgaaattgggatgaagggtgaaaggcggaggggcaaaggacacgacggactgtatcagggactgtccacagccacaaaagatacctatgacgcactccatatgcaggctctcccacccagatga

SEQ ID NO: 133 - DNA, 3068 bp, Artificial Sequence
  Synthetic Sequence
tcgagatggctctgcctgttacagctctgctgctgcctctggctctgcttctgcatgctgctagacctcaggtgcagctggttcagtctggcgccgaagtgaagaaacctggcagcagcgtgaaggtgtcctgcaaggccagcggctacacctttaccgactacaacatgcactgggtccgacaggcccctggacaaggacttgagtggatcggctacatctacccctacaatggcggcaccggctacaaccagaagttcaagagcaaggccaccatcaccgccgacgagagcacaaacaccgcctacatggaactgagcagcctgagaagcgaggacaccgccgtgtactactgcgctagaggcagacccgccatggattattggggccagggaaccctggtcaccgtttctagcggaggcggaggatctggtggcggaggaagtggcggaggcggtagcgatattcagatgacacagagccccagcagcctgtctgccagcgtgggagatagagtgaccatcacctgtagagccagcgagagcgtggacaactacggcatcagcttcatgaactggttccagcagaagcccggcaaggcccctaagctgctgatctacgccgccagcaatcaaggcagcggagtgcctagcagattttccggctctggcagcggcaccgatttcaccctgaccatcagtagcctgcagcctgacgacttcgccacctactactgccagcagagcaaagaggtgccctggacatttggacagggcaccaaggtggaaatcaagtccggaatcgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctacagaagcgatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaggatccaggaagagaagaggctctggtacccccgatccttggggcagcggcgccacaaacttctctctgctaaagcaagcaggtgatgttgaagaaaaccccgggcctcttgagatgctgctggtcacatctctgctgctgtgcgagctgccccatcctgcctttctgctgattcctgaggtgcagctggtgcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacaagagcacaagcaccgcctacatggaactgagcagcctgagaagcgaggacaccgccgtgtactactgcgccacatttgccctgttcggcttcagagagcaggccttcgatatttggggccagggcaccaccgtgacagtttctagcggaggcggaggatctggtggcggaggaagtggcggaggcggtagcgatattcagatgacacagagccccagcagcctgtctgcctctgtgggagacagagtgacaattacctgccgggccagccagagcatcagctcctacctgaactggtatcagcaaaagcccggcaaggcccctaagctgctgatctatgctgcctccagtctgcagagcggcgtgccaagcagattttctggcagcggctctggcaccgacttcaccctgaccatatctagcctgcagccagaggacctggccacctactactgtcagcagagctacagcacccctttcaccttcggacctggcaccaaggtggacatcaagagcggcactagtaccaccacaccagctcctagacctccaactcctgctcctacaatcgccagccagcctctgtctctgaggccagaagcctgtagacctgctgctggcggagccgtgcatacaagaggactggatttcgcctgcgacatctacatctgggctcctctggccggaacatgcggagtgctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgtctagacgcgtgaagttctccagatccgctgatgctcccgcttatcagcaggggcagaatcagctctataatgaactgaatctggggcggagagaggaatacgacgtcctcgataagaggcgcggcagggaccctgagatgggaggaaaacctcggagaaaaaaccctcaggaagggctctacaacgagctgcagaaagataagatggctgaagcttactccgaaattgggatgaagggtgaaaggcggaggggcaaaggacacgacggactgtatcagggactgtccacagccacaaaagatacctatgacgcactccatatgcaggctctcccacccagatgagtcgac

SEQ ID NO: 134 - DNA, 3084 bp, Artificial Sequence
  Synthetic Sequence
atggaattcggcctgagctggctgttcctggtggccattctgaagggcgtgcagtgcagcagagacatccagatgacccagaccaccagcagcctgtctgcctctctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggacattgccacctacttttgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttcttccggaaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtatgcataagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaggatccaggaagagaagaggctctggtacccccgatccttggggcagcggcgccacaaacttctctctgctaaagcaagcaggtgatgttgaagaaaaccccgggcctcttgagatgctgctggtcacatctctgctgctgtgcgagctgccccatcctgcctttctgctgattcctgaggtgcagctggtgcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacaagagcacaagcaccgcctacatggaactgagcagcctgagaagcgaggacaccgccgtgtactactgcgccacatttgccctgttcggcttcagagagcaggccttcgatatttggggccagggcaccaccgtgacagtttctagcggaggcggaggatctggtggcggaggaagtggcggaggcggtagcgatattcagatgacacagagccccagcagcctgtctgcctctgtgggagacagagtgacaattacctgccgggccagccagagcatcagctcctacctgaactggtatcagcaaaagcccggcaaggcccctaagctgctgatctatgctgcctccagtctgcagagcggcgtgccaagcagattttctggcagcggctctggcaccgacttcaccctgaccatatctagcctgcagccagaggacctggccacctactactgtcagcagagctacagcacccctttcaccttcggacctggcaccaaggtggacatcaagagcggcactagtaccaccacaccagctcctagacctccaactcctgctcctacaatcgccagccagcctctgtctctgaggccagaagcctgtagacctgctgctggcggagccgtgcatacaagaggactggatttcgcctgcgacatctacatctgggctcctctggccggaacatgcggagtgctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgtctagacgcgtgaagttctccagatccgctgatgctcccgcttatcagcaggggcagaatcagctctataatgaactgaatctggggcggagagaggaatacgacgtcctcgataagaggcgcggcagggaccctgagatgggaggaaaacctcggagaaaaaaccctcaggaagggctctacaacgagctgcagaaagataagatggctgaagcttactccgaaattgggatgaagggtgaaaggcggaggggcaaaggacacgacggactgtatcagggactgtccacagccacaaaagatacctatgacgcactccatatgcaggctctcccacccagatga

SEQ ID NO: 135 - DNA, 3095 bp, Artificial Sequence
  Synthetic Sequence
tcgagatggaattcggcctgagctggctgttcctggtggccattctgaagggcgtgcagtgcagcagagacatccagatgacccagaccaccagcagcctgtctgcctctctgggcgatagagtgaccatcagctgtagagccagccaggacatcagcaagtacctgaactggtatcagcagaaacccgacggcaccgtgaagctgctgatctaccacaccagcagactgcacagcggcgtgccaagcagattttctggcagcggctctggcaccgactacagcctgaccatctccaacctggaacaagaggacattgccacctacttttgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttcttccggaaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtatgcataagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaggatccaggaagagaagaggctctggtacccccgatccttggggcagcggcgccacaaacttctctctgctaaagcaagcaggtgatgttgaagaaaaccccgggcctcttgagatgctgctggtcacatctctgctgctgtgcgagctgccccatcctgcctttctgctgattcctgaggtgcagctggtgcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacaagagcacaagcaccgcctacatggaactgagcagcctgagaagcgaggacaccgccgtgtactactgcgccacatttgccctgttcggcttcagagagcaggccttcgatatttggggccagggcaccaccgtgacagtttctagcggaggcggaggatctggtggcggaggaagtggcggaggcggtagcgatattcagatgacacagagccccagcagcctgtctgcctctgtgggagacagagtgacaattacctgccgggccagccagagcatcagctcctacctgaactggtatcagcaaaagcccggcaaggcccctaagctgctgatctatgctgcctccagtctgcagagcggcgtgccaagcagattttctggcagcggctctggcaccgacttcaccctgaccatatctagcctgcagccagaggacctggccacctactactgtcagcagagctacagcacccctttcaccttcggacctggcaccaaggtggacatcaagagcggcactagtaccaccacaccagctcctagacctccaactcctgctcctacaatcgccagccagcctctgtctctgaggccagaagcctgtagacctgctgctggcggagccgtgcatacaagaggactggatttcgcctgcgacatctacatctgggctcctctggccggaacatgcggagtgctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgtctagacgcgtgaagttctccagatccgctgatgctcccgcttatcagcaggggcagaatcagctctataatgaactgaatctggggcggagagaggaatacgacgtcctcgataagaggcgcggcagggaccctgagatgggaggaaaacctcggagaaaaaaccctcaggaagggctctacaacgagctgcagaaagataagatggctgaagcttactccgaaattgggatgaagggtgaaaggcggaggggcaaaggacacgacggactgtatcagggactgtccacagccacaaaagatacctatgacgcactccatatgcaggctctcccacccagatgagtcgac

SEQ ID NO: 136 - DNA, 3075 bp, Artificial Sequence
  Synthetic Sequence
atggctctgcctgttacagctctgctgctgcctctggctctgcttctgcatgctgctagacctcaggtgcagctggttcagtctggcgccgaagtgaagaaacctggcagcagcgtgaaggtgtcctgcaaggccagcggctacacctttaccgactacaacatgcactgggtccgacaggcccctggacaaggacttgagtggatcggctacatctacccctacaatggcggcaccggctacaaccagaagttcaagagcaaggccaccatcaccgccgacgagagcacaaacaccgcctacatggaactgagcagcctgagaagcgaggacaccgccgtgtactactgcgctagaggcagacccgccatggattattggggccagggaaccctggtcaccgtttctagcggaggcggaggatctggtggcggaggaagtggcggaggcggtagcgatattcagatgacacagagccccagcagcctgtctgccagcgtgggagatagagtgaccatcacctgtagagccagcgagagcgtggacaactacggcatcagcttcatgaactggttccagcagaagcccggcaaggcccctaagctgctgatctacgccgccagcaatcaaggcagcggagtgcctagcagattttccggctctggcagcggcaccgatttcaccctgaccatcagtagcctgcagcctgacgacttcgccacctactactgccagcagagcaaagaggtgccctggacatttggacagggcaccaaggtggaaatcaagtccggaaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtatgcataagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaggatccaggaagagaagaggctctggtacccccgatccttggggcagcggcgccacaaacttctctctgctaaagcaagcaggtgatgttgaagaaaaccccgggcctcttgagatgctgctggtcacatctctgctgctgtgcgagctgccccatcctgcctttctgctgattcctgaggtgcagctggtgcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacaagagcacaagcaccgcctacatggaactgagcagcctgagaagcgaggacaccgccgtgtactactgcgccacatttgccctgttcggcttcagagagcaggccttcgatatttggggccagggcaccaccgtgacagtttctagcggaggcggaggatctggtggcggaggaagtggcggaggcggtagcgatattcagatgacacagagccccagcagcctgtctgcctctgtgggagacagagtgacaattacctgccgggccagccagagcatcagctcctacctgaactggtatcagcaaaagcccggcaaggcccctaagctgctgatctatgctgcctccagtctgcagagcggcgtgccaagcagattttctggcagcggctctggcaccgacttcaccctgaccatatctagcctgcagccagaggacctggccacctactactgtcagcagagctacagcacccctttcaccttcggacctggcaccaaggtggacatcaagagcggcactagtaccaccacaccagctcctagacctccaactcctgctcctacaatcgccagccagcctctgtctctgaggccagaagcctgtagacctgctgctggcggagccgtgcatacaagaggactggatttcgcctgcgacatctacatctgggctcctctggccggaacatgcggagtgctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgtctagacgcgtgaagttctccagatccgctgatgctcccgcttatcagcaggggcagaatcagctctataatgaactgaatctggggcggagagaggaatacgacgtcctcgataagaggcgcggcagggaccctgagatgggaggaaaacctcggagaaaaaaccctcaggaagggctctacaacgagctgcagaaagataagatggctgaagcttactccgaaattgggatgaagggtgaaaggcggaggggcaaaggacacgacggactgtatcagggactgtccacagccacaaaagatacctatgacgcactccatatgcaggctctcccacccagatga

SEQ ID NO: 137 - DNA, 3086 bp, Artificial Sequence
  Synthetic Sequence
tcgagatggctctgcctgttacagctctgctgctgcctctggctctgcttctgcatgctgctagacctcaggtgcagctggttcagtctggcgccgaagtgaagaaacctggcagcagcgtgaaggtgtcctgcaaggccagcggctacacctttaccgactacaacatgcactgggtccgacaggcccctggacaaggacttgagtggatcggctacatctacccctacaatggcggcaccggctacaaccagaagttcaagagcaaggccaccatcaccgccgacgagagcacaaacaccgcctacatggaactgagcagcctgagaagcgaggacaccgccgtgtactactgcgctagaggcagacccgccatggattattggggccagggaaccctggtcaccgtttctagcggaggcggaggatctggtggcggaggaagtggcggaggcggtagcgatattcagatgacacagagccccagcagcctgtctgccagcgtgggagatagagtgaccatcacctgtagagccagcgagagcgtggacaactacggcatcagcttcatgaactggttccagcagaagcccggcaaggcccctaagctgctgatctacgccgccagcaatcaaggcagcggagtgcctagcagattttccggctctggcagcggcaccgatttcaccctgaccatcagtagcctgcagcctgacgacttcgccacctactactgccagcagagcaaagaggtgccctggacatttggacagggcaccaaggtggaaatcaagtccggaaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtatgcataagaggggcagaaagaagctgctctacatcttcaagcagccctttatgagacccgtgcagacaacccaggaggaagacggatgcagctgcaggttccctgaggaggaggagggcggctgcgaactggatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaggatccaggaagagaagaggctctggtacccccgatccttggggcagcggcgccacaaacttctctctgctaaagcaagcaggtgatgttgaagaaaaccccgggcctcttgagatgctgctggtcacatctctgctgctgtgcgagctgccccatcctgcctttctgctgattcctgaggtgcagctggtgcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacaagagcacaagcaccgcctacatggaactgagcagcctgagaagcgaggacaccgccgtgtactactgcgccacatttgccctgttcggcttcagagagcaggccttcgatatttggggccagggcaccaccgtgacagtttctagcggaggcggaggatctggtggcggaggaagtggcggaggcggtagcgatattcagatgacacagagccccagcagcctgtctgcctctgtgggagacagagtgacaattacctgccgggccagccagagcatcagctcctacctgaactggtatcagcaaaagcccggcaaggcccctaagctgctgatctatgctgcctccagtctgcagagcggcgtgccaagcagattttctggcagcggctctggcaccgacttcaccctgaccatatctagcctgcagccagaggacctggccacctactactgtcagcagagctacagcacccctttcaccttcggacctggcaccaaggtggacatcaagagcggcactagtaccaccacaccagctcctagacctccaactcctgctcctacaatcgccagccagcctctgtctctgaggccagaagcctgtagacctgctgctggcggagccgtgcatacaagaggactggatttcgcctgcgacatctacatctgggctcctctggccggaacatgcggagtgctgctgctgagcctggtcatcaccctgtactgcaagcggggcagaaagaagctgctgtacatcttcaagcagcccttcatgcggcccgtgcagaccacacaagaggaagatggctgctcctgcagattccccgaggaagaagaaggcggctgcgagctgtctagacgcgtgaagttctccagatccgctgatgctcccgcttatcagcaggggcagaatcagctctataatgaactgaatctggggcggagagaggaatacgacgtcctcgataagaggcgcggcagggaccctgagatgggaggaaaacctcggagaaaaaaccctcaggaagggctctacaacgagctgcagaaagataagatggctgaagcttactccgaaattgggatgaagggtgaaaggcggaggggcaaaggacacgacggactgtatcagggactgtccacagccacaaaagatacctatgacgcactccatatgcaggctctcccacccagatgagtcgac

SEQ ID NO: 138 - protein, 1021 aa, Artificial Sequence
  Synthetic Sequence
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSRKRRGSGTPDPWGSGATNFSLLKQAGDVEENPGPLEMLLVTSLLLCELPHPAFLLIPEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKSGTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 489 aa, Artificial Sequence
  Synthetic Sequence
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 494 aa, Artificial Sequence
  Synthetic Sequence
MLLVTSLLLCELPHPAFLLIPEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKSGTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 468 aa, Artificial Sequence
  Synthetic Sequence
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 473 aa, Artificial Sequence
  Synthetic Sequence
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKSGTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 1018 aa, Artificial Sequence
  Synthetic Sequence
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSRKRRGSGTPDPWGSGATNFSLLKQAGDVEENPGPLEMLLVTSLLLCELPHPAFLLIPEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKSGTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 486 aa, Artificial Sequence
  Synthetic Sequence
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 494 aa, Artificial Sequence; identical to SEQ ID NO: 140
  Synthetic Sequence

SEQ ID NO: 146 - protein, 465 aa, Artificial Sequence
  Synthetic Sequence
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 473 aa, Artificial Sequence; identical to SEQ ID NO: 142
  Synthetic Sequence

SEQ ID NO: 148 - protein, 1027 aa, Artificial Sequence
  Synthetic Sequence
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCMHKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSRKRRGSGTPDPWGSGATNFSLLKQAGDVEENPGPLEMLLVTSLLLCELPHPAFLLIPEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKSGTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 495 aa, Artificial Sequence
  Synthetic Sequence
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCMHKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 494 aa, Artificial Sequence; identical to SEQ ID NO: 140
  Synthetic Sequence

SEQ ID NO: 151 - protein, 474 aa, Artificial Sequence
  Synthetic Sequence
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCMHKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - protein, 473 aa, Artificial Sequence; identical to SEQ ID NO: 142
  Synthetic Sequence

SEQ ID NO: 153 - protein, 1024 aa, Artificial Sequence
  Synthetic Sequence
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGTTTPAPRPPTPAPTIASQPLSLRPEACRPAAGGAVHTRGLDFACDIYIWAPLAGTCGVLLLSLVITLYCMHKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSRKRRGSGTPDPWGSGATNFSLLKQAGDVEENPGPLEMLLVTSLLLCELPHPAFLLIPEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKSGTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELSRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 492 aa, Artificial Sequence
  Synthetic Sequence
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGTTTPAPRPPTPAPTIASQPLSLRPEACRPAAGGAVHTRGLDFACDIYIWAPLAGTCGVLLLSLVITLYCMHKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 494 aa, Artificial Sequence; identical to SEQ ID NO: 140
  Synthetic Sequence

SEQ ID NO: 156 - protein, 471 aa, Artificial Sequence
  Synthetic Sequence
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGTTTPAPRPPTPAPTIASQPLSLRPEACRPAAGGAVHTRGLDFACDIYIWAPLAGTCGVLLLSLVITLYCMHKRGRKKLLYIFKQPFMRPVQTTQEEDGCSCRFPEEEEGGCELDIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 473 aa, Artificial Sequence; identical to SEQ ID NO: 142
  Synthetic Sequence